(19) United States
(12) Patent Application Publication
(10) Pub. No.: US 2024/0228960 A1
(43) Pub. Date: Jul. 11, 2024

(54) METHODS AND COMPOSITIONS FOR REDUCING IMMUNE CELL EXHAUSTION USING MITOCHONDRIA REPLACEMENT

(71) Applicant: Imel Biotherapeutics, Inc., Waltham, MA (US)

(72) Inventors: Satoshi GOJO, Kyoto-Fu (JP); Daisuke KAMI, Kyoto-Fu (JP)

(21) Appl. No.: 18/562,816

(22) PCT Filed: May 18, 2022
(86) PCT No.: PCT/IB2022/054638

§ 371 Date: Nov. 20, 2023

Related U.S. Application Data
(60) Provisional application No. 63/190,115, filed on May 18, 2021.

(51) Int. Cl. C12N 5/0783; A61K 39/00

(52) U.S. Cl. CPC C12N 5/0636; A61K 39/4611; A61K 39/4631; A61K 39/464422; C12N 2501/515; C12N 2510/00

(57) ABSTRACT

The present disclosure provides methods and compositions for producing mitochondria replaced T cells from exhausted T cells, that involves reducing exhausted T cells mitochondrial DNA (mtDNA) and incubating with isolated exogenous mitochondria for a sufficient period of time to generate mitochondria replaced T cells in which expression of at least one exhaustion marker is altered by at least 5%, at least 10%, 20% (e.g., at least 1.25 fold), at least 30%, at least 40%, at least 50%, at least 60%, or more, wherein the mitochondria replaced T cells have improved effector function relative to the exhausted T cells. In addition, the present disclosure also provides methods of treating or ameliorating an age-related disease (e.g., cancer or an autoimmune disease), as well as methods for ameliorating a symptom of a chronic infection (e.g., a chronic viral infection), that involve administering the mitochondria replaced T cells.

This application claims the benefit of U.S. Provisional Application No. 63/190,115, filed May 18, 2021, which is incorporated herein by reference in its entirety.

### 1. SEQUENCE LISTING

The instant application contains a Sequence Listing which is submitted electronically in ASCII format and is hereby incorporated by reference in its entirety. Said ASCII copy, created on May 18, 2022, is named 14595-009-228_SL.txt and is 2,410 bytes in size.

### 2. FIELD

The present disclosure relates, in part, to methods and compositions for reducing immune cell exhaustion using mitochondria replacement. In a specific aspect, the disclosure relates to methods for producing mitochondria replaced T cells from exhausted T cells comprising: (a) electroporating exhausted T cells with a nucleic acid sequence comprising a nucleotide sequence encoding XbaIR (e.g., a fusion protein comprising a mitochondrial-targeted sequence (MTS) and XbaIR) to reduce endogenous mitochondrial DNA (mtDNA) copy number; and (b) incubating the exhausted T cells having reduced mitochondria DNA (mtDNA) copy number with isolated exogenous mitochondria for a sufficient period of time to generate a mitochondria replaced T cell using such that the expression of an exhaustion marker (e.g., programmed cell death-1 (PD-1)) is decreased by at least 5%, at least 10%, at least 20% (e.g., at least 1.25 fold), at least 30%, at least 40%, at least 50%, at least 60%, or more in the mitochondria replaced T cells relative to the exhausted T cell. The disclosure also relates to therapeutic methods for treating diseases (e.g., cancer) and chronic viral infections using such mitochondrial replaced T cells.

### 3. BACKGROUND

T cell exhaustion generally involves a state of T cell dysfunction that arises from chronic antigen stimulation, such as in the setting of chronic viral infection and cancer (see Wherry, E. (2011) Nat Immunol 12, 492-499). Hallmarks of T cell exhaustion include poor effector function, sustained expression of inhibitory receptors and/or a transcriptional state distinct from that of functional effector or memory T cells. Exhaustion prevents optimal control of infection and tumors.

Cancer immunotherapy has recently emerged as a promising strategy for enlisting and strengthening the power of a patient's immune system to attack tumors. One immunotherapy approach involves collecting and engineering patients' own T cells to treat their cancer. Two exemplary approaches for engineering T cells include engineering patients' T cells to express a specific T-cell receptor (TCR) or engineering T cells to express chimeric antigen receptors (CARs). TCRs use naturally occurring receptors that can also recognize antigens that are inside tumor cells. Small pieces of these antigens are shuttled to the cell surface and “presented” to the immune system as part of a collection of proteins called the MHC complex. CARs, on the other hand, comprise portions of an antibody that can recognize a specific antigen on the surface of cancer cells. Despite their potential as a therapy, CAR-T cells also become exhausted and can require immune checkpoint blockade so that they can restore their functionality.

T-cell exhaustion has also been reported in various human chronic viral infections such as human immunodeficiency virus (HIV), hepatitis B (HBV), and hepatitis C (HCV). It also develops more generally under conditions of antigen persistence that arise during certain non-viral infections such as malaria and Mycobacterium tuberculosis.

Thus, there is a significant unmet need to reduce and/or reverse T cell exhaustion in settings of chronic antigen stimulation.

### 4. SUMMARY

In one aspect, provided herein is a method for producing mitochondria replaced T cells from exhausted T cells, the method comprising incubating exhausted T cells having reduced mitochondria DNA (mtDNA) with isolated exogenous mitochondria for a sufficient period of time to generate mitochondria replaced T cells in which the mitochondria replaced T cells have decreased expression of one, two or more markers of T cell exhaustion (e.g., PD-1 expression) relative to the expression of one, two or more markers of T cell exhaustion by the exhausted T cells from which the mitochondria replaced T cells were produced. In specific embodiments, the decrease in expression of one, two more markers of T cell exhaustion by mitochondria replaced T cells is indicative of an improvement of one, two or more effector functions of the T cells. In a specific embodiment, provided herein is a method for producing mitochondria replaced T cells from exhausted T cells, the method comprising incubating exhausted T cells having reduced endogenous mitochondria DNA (mtDNA) copy number with isolated exogenous mitochondria for a sufficient period of time to generate mitochondria replaced T cells in which the expression of programmed cell death-1 (PD-1) is decreased by at least 5%, at least 10%, 20% (e.g., at least 1.25 fold), at least 30%, at least 40%, at least 50%, at least 60%, or more relative to expression of the PD-1 by the exhausted T cells from which the mitochondria replaced T cells were produced. In another embodiment, provided herein is a method for producing mitochondria replaced T cells from exhausted T cells, the method comprising: (a) electroporating exhausted T cells with a nucleic acid sequence comprising a nucleotide sequence encoding XbaIR to reduce endogenous mitochondrial DNA (mtDNA) copy number; and (b) incubating the exhausted T cells having reduced endogenous mitochondria DNA (mtDNA) copy number with isolated exogenous mitochondria for a sufficient period of time to generate a mitochondria replaced T cell in which the expression of programmed cell death-1 (PD-1) is decreased by at least 5%, at least 10%, at least 20% (e.g., at least 1.25 fold), at least 30%, at least 40%, at least 50%, at least 60%, or more relative to the expression of PD-1 by the exhausted T cells from which the mitochondria replaced T cells were produced. In another embodiment, provided herein is a method for producing mitochondria replaced T cells from exhausted T cells, the method comprising: (a) electroporating exhausted T cells with a nucleic acid sequence comprising a nucleotide sequence encoding a fusion protein comprising a mitochondrial-targeted sequence (MTS) and XbaIR to reduce endogenous mitochondrial DNA (mtDNA) copy number; and (b) incubating the exhausted T cells having reduced endogenous mitochondria DNA (mtDNA) copy number with isolated exogenous mitochondria for a sufficient period of time to generate a mitochondria replaced T cell in which the expression of programmed cell death-1 (PD-1) is decreased by at least 5%, at least 10%, at least 20% (e.g., at least 1.25 fold), at least 30%, at least 40%, at least 50%, at least 60%, or more relative to the expression of PD-1 by the exhausted T cells from which the mitochondria replaced T cells were produced. In some embodiments, the nucleic acid sequence is RNA (e.g., mRNA). In some embodiments, the nucleic acid sequence is DNA (e.g., cDNA).

In a specific aspect, provided herein is a method for producing mitochondria replaced T cells from exhausted T cells, the method comprising incubating exhausted T cells having reduced endogenous mitochondria DNA (mtDNA) copy number with isolated exogenous mitochondria for a sufficient period of time to generate mitochondria replaced T cells in which the mitochondria replaced T cells have improved effector function relative to the exhausted T cells. In a specific embodiment, provided herein is a method for producing mitochondria replaced T cells from exhausted T cells, the method comprising incubating exhausted T cells having reduced endogenous mitochondria DNA (mtDNA) copy number with isolated exogenous mitochondria for a sufficient period of time to generate mitochondria replaced T cells in which expression of programmed cell death-1 (PD-1) is decreased by at least 5%, at least 10%, at least 20% (e.g., at least 1.25 fold), at least 30%, at least 40%, at least 50%, at least 60%, or more relative to the expression of PD-1 by the exhausted T cells from which the mitochondria replaced T cells were produced, wherein the mitochondria replaced T cells have improved effector function relative to the exhausted T cells. In some embodiments, the nucleic acid sequence is RNA (e.g., mRNA). In some embodiments, the nucleic acid sequence is DNA (e.g., cDNA).

In another embodiment, provided herein is a method for producing mitochondria replaced T cells from exhausted T cells, the method comprising: (a) electroporating exhausted T cells with a nucleic acid sequence comprising a nucleotide sequence encoding XbaIR (in specific embodiments, the nucleic acid sequence comprises a nucleotide sequence encoding a fusion protein comprising a mitochondrial-targeted sequence (MTS) and XbaIR) to reduce endogenous mitochondrial DNA (mtDNA) copy number; and (b) incubating the exhausted T cells having reduced endogenous mitochondria DNA (mtDNA) copy number with isolated exogenous mitochondria for a sufficient period of time to generate a mitochondria replaced T cell in which the mitochondria replaced T cells have improved effector function relative to the exhausted T cells. In a specific embodiment, provided herein is a method for producing mitochondria replaced T cells from exhausted T cells, the method comprising: (a) electroporating exhausted T cells with a nucleic acid sequence comprising a nucleotide sequence encoding XbaIR (in specific embodiments, the nucleic acid sequence comprises a nucleotide sequence encoding a fusion protein comprising a mitochondrial-targeted sequence (MTS) and XbaIR) to reduce endogenous mitochondrial DNA (mtDNA) copy number; and (b) incubating the exhausted T cells having reduced endogenous mitochondria DNA (mtDNA) copy number with isolated exogenous mitochondria for a sufficient period of time to generate a mitochondria replaced T cell in which expression of PD-1 is decreased by at least 5%, at least 10%, at least 20% (e.g., at least 1.25 fold), at least 30%, at least 40%, at least 50%, at least 60%, or more relative to the expression of PD-1 by the exhausted T cells from which the mitochondria replaced T cells were produced, wherein the mitochondria replaced T cells have improved effector function relative to the exhausted T cells. In some embodiments, the nucleic acid sequence is RNA (e.g., mRNA). In some embodiments, the nucleic acid sequence is DNA (e.g., cDNA).

In some embodiments, expression of PD-1 is decreased by approximately 5% to approximately 60%, approximately 10% to approximately 50%, approximately 20% to approximately 50%, approximately 10% to approximately 40%, approximately 10% to approximately 30%, approximately 10% to approximately 20%, approximately 20% to approximately 40%, or approximately 20% to approximately 30%, relative to the expression of PD-1 by the exhausted T cells from which the mitochondria replaced T cells were produced.

In certain embodiments, the incubation of the exhausted T cells with the isolated exogenous mitochondria occurs in the presence of rapamycin. In specific embodiments, rapamycin is present at a concentration of 100 nM to 1000 nM.

In certain embodiments, the expression of PD-1 by mitochondria replaced T cells is decreased by at least 1.1 fold relative to the expression of PD-1 by the exhausted T cells from which the mitochondria replaced T cells were produced. In certain embodiments, the expression of PD-1 by mitochondria replaced T cells is decreased by at least 1.15 fold relative to the expression of PD-1 by the exhausted T cells from which the mitochondria replaced T cells were produced. In certain embodiments, the expression of PD-1 by mitochondria replaced T cells is decreased by at least 1.20 fold relative to the expression of PD-1 by the exhausted T cells from which the mitochondria replaced T cells were produced. In certain embodiments, the expression of PD-1 by mitochondria replaced T cells is decreased by at least 1.25 fold relative to the expression of PD-1 by the exhausted T cells from which the mitochondria replaced T cells were produced. In certain embodiments, the expression of PD-1 by mitochondria replaced T cells is decreased by at least 1.3 fold relative to the expression of PD-1 by the exhausted T cells from which the mitochondria replaced T cells were produced. In certain embodiments, the expression of PD-1 by mitochondria replaced T cells is decreased by at least 1.4 fold relative to the expression of PD-1 by the exhausted T cells from which the mitochondria replaced T cells were produced. In certain embodiments, the expression of PD-1 by mitochondria replaced T cells is decreased by at least 1.5 fold relative to the expression of PD-1 by the exhausted T cells from which the mitochondria replaced T cells were produced. In some embodiments, the expression of PD-1 by mitochondria replaced T cells is decreased by at least 2 fold relative to the expression of PD-1 by the exhausted T cells from which the mitochondria replaced T cells were produced. In some embodiments, expression of PD-1 by mitochondria replaced T cells is decreased by at least 5 fold relative to the expression of PD-1 by the exhausted T cells from which the mitochondria replaced T cells were produced. In certain embodiments, the expression of PD-1 by mitochondria replaced T cells is decreased by at least 5% relative to the expression of PD-1 by the exhausted T cells from which the mitochondria replaced T cells were produced. In certain embodiments, the expression of PD-1 by mitochondria replaced T cells is decreased by at least 10% relative to the expression of PD-1 by the exhausted T cells from which the mitochondria replaced T cells were produced. In certain embodiments, the expression of PD-1 by mitochondria replaced T cells is decreased by at least 15% relative to the expression of PD-1 by the exhausted T cells from which the mitochondria replaced T cells were produced. In certain embodiments, the expression of PD-1 by mitochondria replaced T cells is decreased by at least 20% relative to the expression of PD-1 by the exhausted T cells from which the mitochondria replaced T cells were produced. In certain embodiments, the expression of PD-1 by mitochondria replaced T cells is decreased by at least 25% relative to the expression of PD-1 by the exhausted T cells from which the mitochondria replaced T cells were produced. In certain embodiments, the expression of PD-1 by mitochondria replaced T cells is decreased by at least 30% relative to the expression of PD-1 by the exhausted T cells from which the mitochondria replaced T cells were produced. In certain embodiments, the expression of PD-1 by mitochondria replaced T cells is decreased by at least 35% relative to the expression of PD-1 by the exhausted T cells from which the mitochondria replaced T cells were produced. In certain embodiments, the expression of PD-1 by mitochondria replaced T cells is decreased by at least 40% relative to the expression of PD-1 by the exhausted T cells from which the mitochondria replaced T cells were produced. In certain embodiments, the expression of PD-1 by mitochondria replaced T cells is decreased by at least 45% relative to the expression of PD-1 by the exhausted T cells from which the mitochondria replaced T cells were produced. In certain embodiments, the expression of PD-1 by mitochondria replaced T cells is decreased by at least 50% relative to the expression of PD-1 by the exhausted T cells from which the mitochondria replaced T cells were produced. In certain embodiments, the expression of PD-1 by mitochondria replaced T cells is decreased by at least 60% relative to the expression of PD-1 by the exhausted T cells from which the mitochondria replaced T cells were produced. In some embodiments, expression of PD-1 by mitochondria replaced T cells is decreased by approximately 5% to approximately 60% relative to the expression of PD-1 by the exhausted T cells from which the mitochondria replaced T cells were produced. In some embodiments, expression of PD-1 by mitochondria replaced T cells is decreased by approximately 10% to approximately 50%, relative to the expression of PD-1 by the exhausted T cells from which the mitochondria replaced T cells were produced. In some embodiments, expression of PD-1 by mitochondria replaced T cells is decreased by approximately 20% to approximately 50%, relative to the expression of PD-1 by the exhausted T cells from which the mitochondria replaced T cells were produced. In some embodiments, expression of PD-1 by mitochondria replaced T cells is decreased by approximately 10% to approximately 40%, relative to the expression of PD-1 by the exhausted T cells from which the mitochondria replaced T cells were produced. In some embodiments, expression of PD-1 by mitochondria replaced T cells is decreased by approximately 10% to approximately 30%, relative to the expression of PD-1 by the exhausted T cells from which the mitochondria replaced T cells were produced. In some embodiments, expression of PD-1 by mitochondria replaced T cells is decreased by approximately 10% to approximately 20%, relative to the expression of PD-1 by the exhausted T cells from which the mitochondria replaced T cells were produced. In some embodiments, expression of PD-1 by mitochondria replaced T cells is decreased by approximately 20% to approximately 40%, relative to the expression of PD-1 by the exhausted T cells from which the mitochondria replaced T cells were produced. In some embodiments, expression of PD-1 by mitochondria replaced T cells is decreased by approximately 20% to approximately 30%, relative to the expression of PD-1 by the exhausted T cells from which the mitochondria replaced T cells were produced. In certain embodiments, the expression of PD-1 by mitochondria replaced T cells is decreased by approximately 1.1 fold to approximately 2.0 fold, relative to the expression of PD-1 by the exhausted T cells from which the mitochondria replaced T cells were produced. In certain embodiments, the expression of PD-1 by mitochondria replaced T cells is decreased by approximately 1.1 fold to approximately 1.75 fold, relative to the expression of PD-1 by the exhausted T cells from which the mitochondria replaced T cells were produced. In certain embodiments, the expression of PD-1 by mitochondria replaced T cells is decreased by approximately 1.1 fold to approximately 1.5 fold, relative to the expression of PD-1 by the exhausted T cells from which the mitochondria replaced T cells were produced. In certain embodiments, the expression of PD-1 by mitochondria replaced T cells is decreased by approximately 1.1 fold to approximately 1.25 fold, relative to the expression of PD-1 by the exhausted T cells from which the mitochondria replaced T cells were produced. In certain embodiments, the expression of PD-1 by mitochondria replaced T cells is decreased by approximately 1.25 fold to approximately 2.0 fold, relative to the expression of PD-1 by the exhausted T cells from which the mitochondria replaced T cells were produced. In certain embodiments, the expression of PD-1 by mitochondria replaced T cells is decreased by approximately 1.25 fold to approximately 5.0 fold, relative to the expression of PD-1 by the exhausted T cells from which the mitochondria replaced T cells were produced. In certain embodiments, the expression of PD-1 by mitochondria replaced T cells is decreased by approximately 1.25 fold to approximately 1.5 fold, relative to the expression of PD-1 by the exhausted T cells from which the mitochondria replaced T cells were produced.

In some embodiments, the method reduces the expression of PD-1, T-cell immunoglobulin and mucin domain-containing protein 3 (TIM3), lymphocyte-activated gene-3 (LAG3), T Cell immunoglobulin and ITIM domain (TIGIT), TOX, or a combination thereof.

In certain embodiments, the isolated exogenous mitochondria is about 20 μg to about 80 μg protein per 1×10⁶ exhausted T cells.

In some embodiments, the mitochondria replaced T cells comprise at least 20% of the exogenous mtDNA. In certain embodiments, the mitochondria replaced T cells comprises at least 20% of exogenous mtDNA and no more than 80% exogenous mtDNA, as measured by TaqMan Single Nucleotide Polymorphism (SNP) Assay.

In specific embodiments, the sufficient period of time to generate mitochondria replaced T cells is at least approximately 24 hours. In certain embodiments, the sufficient period of time to generate mitochondria replaced T cells is at least 36 hours. In some embodiments, the sufficient period of time to generate mitochondria replaced T cells is at least 48 hours. In certain embodiments, the sufficient period of time to generate mitochondria replaced T cells is about 24 hours to about 48 hours, about 36 hours to 48 hours, about 24 hours to about 72 hours, or about 48 hours to about 72 hours.

In some embodiments, the improved effector function comprises one, two or more of the following, or all of the following: increased proliferation, increased cytotoxicity, or increased secretion of cytokines.

In certain embodiments, the exhausted T cells comprise an exogenous polynucleotide encoding a T cell receptor (TCR) or a chimeric antigen receptor (CAR). In specific embodiments, the exhausted T cells have been genetically modified to express a T cell receptor (TCR) or a chimeric antigen receptor (CAR).

In another aspect, provided herein are mitochondria replaced T cells generated by the method described herein, and compositions comprising such cells.

In another aspect, provided herein is a composition comprising an effective amount of the mitochondria replaced T cell of the present disclosure, and a pharmaceutically acceptable carrier.

In another aspect, provided herein is a method for ameliorating a symptom of a chronic viral infection in a subject in need thereof, comprising administering to the subject a composition that includes an effective amount of the mitochondria replaced T cell of the present disclosure, and a pharmaceutically acceptable carrier. In certain embodiments, the chronic viral infection is a human immunodeficiency virus (HIV) infection, a hepatitis B virus (HBV) infection, a cytomegalovirus infection (CMV), or a Severe acute respiratory syndrome (SARS)-coronavirus (CoV)-2 infection. In a specific embodiment, the subject is human.

In another aspect, provided herein is a method for treating cancer in a subject in need thereof, comprising administering to the subject a composition that includes an effective amount of the mitochondria replaced T cell of the present disclosure, and a pharmaceutically acceptable carrier. In a specific embodiment, the subject is human.

In another aspect, provided herein is a composition comprising an effective amount of a mitochondria replaced T cell comprising an exogenous polynucleotide encoding a T cell receptor (TCR) or a chimeric antigen receptor (CAR), and a pharmaceutically acceptable carrier. In specific embodiments, the mitochondria replaced T cell has been genetically modified to express a T cell receptor (TCR) or a chimeric antigen receptor (CAR).

In another aspect, provided herein is a method for treating a cancer in a subject in need thereof, comprising administering to the subject a composition comprising a mitochondria replaced T cells described herein, wherein the mitochondria replaced T cells comprise an exogenous polynucleotide encoding a T cell receptor (TCR) or a chimeric antigen receptor (CAR), and a pharmaceutically acceptable carrier. In another aspect, provided herein is a method for ameliorating a symptom of a cancer in a subject in need thereof, comprising administering to the subject a composition comprising a mitochondria replaced T cells described herein, wherein the mitochondria replaced T cells comprise an exogenous polynucleotide encoding a T cell receptor (TCR) or a chimeric antigen receptor (CAR), and a pharmaceutically acceptable carrier. In a specific embodiment, the subject is human.

In another aspect, provided herein is a method for treating a disease or condition associated with, involving, or caused by T cell exhaustion in a subject in need thereof, comprising administering to the subject a composition comprising a mitochondria replaced T cells described herein, wherein the mitochondria replaced T cells comprise an exogenous polynucleotide encoding a T cell receptor (TCR) or a chimeric antigen receptor (CAR), and wherein the disease or condition is: (a) cancer; (b) a viral infection (e.g., a chronic viral infection); (c) a bacterial infection; (d) obesity or a metabolic disorder; (e) alcoholism; (f) hypermotility; (g) excessive mental stress; (h) hypoxia; (i) an injury; (j) aging; (k) aging related immunological dysfunction; (l) a fibrotic disease; (m) a macular disease; (n) a muscular degenerative disease; or (o) a neurodegenerative disease. In another aspect, provided herein is a method for ameliorating a symptom of a disease or condition associated with, involving, or caused by T cell exhaustion in a subject in need thereof, comprising administering to the subject a composition comprising a mitochondria replaced T cells described herein, wherein the mitochondria replaced T cells comprise an exogenous polynucleotide encoding a T cell receptor (TCR) or a chimeric antigen receptor (CAR), wherein the disease or condition is: (a) cancer; (b) a viral infection (e.g., a chronic viral infection); (c) a bacterial infection; (d) obesity or a metabolic disorder; (e) alcoholism; (f) hypermotility; (g) excessive mental stress; (h) hypoxia; (i) an injury; (j) aging; (k) aging related immunological dysfunction; (l) a fibrotic disease; (m) a macular disease; (n) a muscular degenerative disease; or (o) a neurodegenerative disease. In a specific embodiment, the subject is human.

In another aspect, provided herein is a method for treating a disease or condition associated with or involving, or caused by T cell exhaustion in a subject in need thereof, comprising administering to the subject a composition comprising a mitochondria replaced T cells described herein, wherein the disease or condition is: (a) CD8+ T cell dysfunction; (b) CD4+ T cell dysfunction; (c) dysfunction in T cell priming; (d) memory T cell dysfunction; (e) effector B cell dysfunction; (f) dysfunction in B cell priming; (g) memory B cell dysfunction; (h) innate lymphoid cell dysfunction; (i) innate T cell dysfunction; (j) innate B cell dysfunction, or (k) a combination of (a) to (j). In another aspect, provided herein is a method for ameliorating a symptom of a disease or condition associated with or involving: (a) CD8+ T cell dysfunction; (b) CD4+ T cell dysfunction; (c) dysfunction in T cell priming; (d) memory T cell dysfunction; (e) effector B cell dysfunction; (f) dysfunction in B cell priming; (g) memory B cell dysfunction; (h) innate lymphoid cell dysfunction; (i) innate T cell dysfunction; (j) innate B cell dysfunction, or (k) a combination of (a) to (j). In a specific embodiment, the subject is human.

### 5. BRIEF DESCRIPTION OF THE FIGURES

FIG. 1. Scheme of Experimental System Illustrating Modulation of human T cell Exhaustion. Co-culture of Ephrin type-B receptor 4 (EPHB4)-specific chimeric antigen receptor (CAR) T cells with human rhabdomyosarcoma cells that constitutively express EPHB4, Rh30, for three days increased PD-1, an exhaustion marker. At day 3, endogenous mitochondrial DNA in the CAR-T cells was depleted using XbaIR mRNA. At day 5, normal human derived fibroblasts (NHDF)-derived donor mitochondria was co-cultured with the CAR-T cells to generate mitochondria replaced (Mir) CAR-T cells (Mir CAR-T cells). At day 7, FACS and TaqMan SNP assays were performed.

FIG. 2A-FIG. 2B. The surrogate marker of mtDNA content, 12S rRNA, was halved on Day 3, confirming that XbaIR caused effective mtDNA reduction (FIG. 2A), and TaqMan SNP assay was performed on Day 7, showing that Mir CAR-T cells contained about 40% NHDF-derived donor mtDNA, whereas the exhausted CAR-T cells and the parental CAR-T cells (termed YG cells) were indistinguishable and both contained a negligible amount of NHDF-derived donor mtDNA (FIG. 2B). Abbreviations: Electroporation (EP); Normal Human Dermal Fibroblast (NHDF).

FIG. 3A-FIG. 3C. FACS analysis on Day 7 of CD3/PD-1 expression in unstained CAR-T cells (FIG. 3A), CAR-T cells as a control (FIG. 3B), and Mir CAR-T cells (FIG. 3C). x-axis is PD-1, y-axis is CD3.

FIG. 4A and FIG. 4B. Expression analysis of PD-1 antigen on day 2 (FIG. 4A), and a bar graph of Mean Fluorescence Intensity (MFI) illustrating that the expression of PD-1 antigen is decreased in Mir CAR-T cells, relative to the parental exhausted CAR-T cells (FIG. 4B).

### 6. DETAILED DESCRIPTION

Provided herein are methods for producing mitochondria replaced T cells from exhausted T cells that involve incubating exhausted T cells having reduced endogenous mitochondria DNA (mtDNA) copy number with isolated exogenous mitochondria for a sufficient period of time to generate mitochondria replaced T cells in which expression of a marker of T cell exhaustion (e.g., programmed cell death-1 (PD-1)) is decreased by at least 5%, at least 10%, at least 20% (e.g., at least 1.25 fold), at least 30%, at least 40%, at least 50%, at least 60%, or more relative to the expression of the marker of T cell exhaustion by the exhausted T cells from which the mitochondria replaced T cells were produced. The mitochondria replaced T cells disclosed herein have utility as a therapy, such as for ameliorating a symptom of a chronic viral infection or a symptom of cancer.

As used herein, the term “mitochondria replaced T cell” is generally intended to mean a T cell in which endogenous mitochondria and/or endogenous mtDNA have been substituted with exogenous mitochondria and/or exogenous mtDNA. In certain embodiments, a mitochondria replaced T cell has all the endogenous mitochondria and/or endogenous mtDNA in a T cell replaced by exogenous mitochondria and/or exogenous mtDNA. In specific embodiments, a mitochondria replaced T cell has endogenous mitochondria replaced with exogenous mitochondria. In such circumstances, the replacement of endogenous mitochondria with exogenous mitochondria is assessed by assessing mtDNA. In specific embodiments, a mitochondria replaced T cell has a certain percentage of endogenous mtDNA replaced with exogenous mtDNA. In some embodiments, a mitochondria replaced T cell has about 5% or more, about 10% or more, about 20% or more, about 30% or more, about 40% or more, about 50% or more, about 60% or more, about 70% or more, about 80% or more, about 90% or more, or about 95% or more of the endogenous mitochondria and/or endogenous mtDNA in a T cell replaced with exogenous mitochondria and/or exogenous mtDNA. In certain embodiments, a mitochondria replaced T cell has about 5% to about 10%, about 10% to about 20%, about 10% to about 30%, about 10% to about 40%, about 20% to about 40%, about 25% to about 50%, about 25% to about 75%, about 50% to about 75%, about 40% to about 50%, about 75% or more to about 85%, about 75% to about 95%, about 30% to about 90%, about 40% to about 90%, about 50% to about 90%, about 30% to about 85%, about 40% to about 85%, about 50% to about 85%, about 30% to about 80%, about 40% to about 80%, about 50% to about 80%, about 30% to about 75%, about 40% to about 75%, about 50% to about 75%, about 60% to about 90%, about 60% to about 85%, about 60% to about 80%, about 60% to about 75% of the endogenous mitochondria and/or endogenous mtDNA in a T cell replaced with exogenous mitochondria and/or exogenous mtDNA. In some embodiments, a mitochondria replaced T cell has at least 5%, at least 10%, at least 15%, at least 20%, at least 30%, at least 40%, at least 50%, at least 60%, at least 70%, at least 80%, at least 90%, or at least 95% of the endogenous mitochondria and/or endogenous mtDNA in a T cell replaced with exogenous mitochondria and/or exogenous mtDNA.

As used herein, the term “isolated” when used in reference to mitochondria generally refers to mitochondria that have been physically separated or removed from the other cellular components of its natural biological environment. In a specific embodiment, a technique described in the Example, infra, is used to isolate mitochondria.

As used herein, the term “isolated” when used in reference to a cell generally means a cell that is substantially free of at least one component as the referenced cell is found in nature. The term includes a cell that is removed from some or all components as it is found in its natural environment. The term also includes a cell that is removed from at least one, some or all components as the cell is found in non-naturally occurring environments. Therefore, an isolated cell is partly or completely separated from other substances as it is found in nature or as it is grown, stored or subsisted in non-naturally occurring environments. Specific examples of isolated cells include partially pure cells (e.g., T cells), and substantially pure cells (e.g., T cells) that are enriched from other cell types (e.g., non-T cells). Accordingly, a referenced cell that is isolated may be 50%, 60%, 70%, 80%, 90%, 95%, 99%, or 100% pure of other cells and/or substances. In a specific embodiment, a technique described in the Example, infra, is used to isolate the referenced cell.

As used herein, the term “exogenous” is generally understood by the skilled person in the art. Generally the term “exogenous” refers to cellular material (e.g., mitochondria or mtDNA) that is not from the recipient cell. For example, exogenous mitochondria or mtDNA may be isolated from a fibroblast introduced into a T cell, such as described in the Example, infra.

As used herein, the term “endogenous” is generally understood by the person skilled in the art. Generally the term “endogenous” refers to cellular material (e.g., mitochondria or mtDNA) that is native to the recipient cell.

As used herein, the term “effective amount” generally refers to the amount of a compound or composition necessary to achieve the desired result(s) under the relevant conditions.

As used herein, the terms “about” or “approximately” when used in conjunction with a number generally refer to any number within 1, 5, 10, 15 or 20% of the referenced number as well as the referenced number.

As used herein, the term “sufficient period of time” generally refers to an amount of time that produces the desired result(s).

As used herein, the term “subject” is generally intended to mean an animal. A subject can be a human or a non-human mammal, such as a dog, cat, bovid, equine, mouse, rat, rabbit, or transgenic species thereof. It is understood that a “subject” can also refer to a “patient,” such as a human patient.

As used herein, the term “XbaIR” has its typical meaning which is the restriction endonuclease XbaI that recognizes and cleaves the following sequence of DNA:

[symbol]

5′ . . . T C T A G A . . . 3′
3′ . . . A G A T C T . . . 5′

[symbol]

As used herein, the term “substantially free” is generally intended to mean an amount that is at or near the threshold of detection of an appropriate assay (e.g., PCR for a nucleotide or an immunoassay for a protein). When used in reference to XbaIR or a nucleotide encoding XbaI (e.g., a fusion protein comprising an MTS and XbaIR), it is generally intended to mean an amount that does not interfere with replacement of mtDNA.

The practice of the embodiments provided herein will employ, unless otherwise indicated, conventional techniques of molecular biology, microbiology, and immunology, which are within the skill of those working in the art. Such techniques are explained fully in the literature. Examples of particularly suitable texts for consultation include the following: Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, M D (1999); Glover, ed., DNA Cloning, Volumes I and II (1985); Gait, ed., Oligonucleotide Synthesis (1984); Hames & Higgins, eds., Nucleic Acid Hybridization (1984); Hames & Higgins, eds., Transcription and Translation (1984); Freshney, ed., Animal Cell Culture: Immobilized Cells and Enzymes (IRL Press, 1986); Immunochemical Methods in Cell and Molecular Biology (Academic Press, London); Scopes, Protein Purification: Principles and Practice (Springer Verlag, N.Y., 2d ed. 1987); and Weir & Blackwell, eds., Handbook of Experimental Immunology, Volumes I-IV (1986).

### 6.1 Method of Producing Mitochondria Replaced T Cells from Exhausted T Cells

The present disclosure is based, in part, on the finding that the introduction of exogenous mitochondria into exhausted T cells can reduce the T cell exhaustion phenotype. Additionally, the present disclosure is based, in part, on the finding that the introduction of exogenous mitochondria into exhausted T cells can improve the effector function of the exhausted T cells. Accordingly, in one aspect, provided herein is a method for producing mitochondria replaced T cells from exhausted T cells, the method comprising incubating exhausted T cells having reduced endogenous mitochondria DNA (mtDNA) copy number with isolated exogenous mitochondria for a sufficient period of time to generate mitochondria replaced T cells in which the mitochondria replaced T cells have an altered (e.g., decreased or increased) level of at least one marker of T cell exhaustion (e.g., PD-1 expression or IL-2 secretion) relative to the level of the at least one marker of T cell exhaustion by the exhausted T cells from which the mitochondria replaced T cells were produced. In certain embodiments, the at least one marker of T cell exhaustion is increased or decreased by at least 0.1 fold relative to the exhausted T cells from which the mitochondria replaced T cells were produced. In certain embodiments, the at least one exhaustion marker is increased or decreased by at least 0.2 fold relative to the exhausted T cells from which the mitochondria replaced T cells were produced. In certain embodiments, the at least one exhaustion marker is increased or decreased by at least 0.3 fold relative to the exhausted T cells from which the mitochondria replaced T cells were produced. In certain embodiments, the at least one exhaustion marker is increased or decreased by at least 0.4 fold relative to the exhausted T cells from which the mitochondria replaced T cells were produced. In certain embodiments, the at least one exhaustion marker is increased or decreased by at least 0.5 fold relative to the exhausted T cells from which the mitochondria replaced T cells were produced. In certain embodiments, the at least one exhaustion marker is increased or decreased by at least 0.6 fold relative to the exhausted T cells from which the mitochondria replaced T cells were produced. In certain embodiments, the at least one exhaustion marker is increased or decreased by at least 0.7 fold relative to the exhausted T cells from which the mitochondria replaced T cells were produced. In certain embodiments, the at least one exhaustion marker is increased or decreased by at least 0.8 fold relative to the exhausted T cells from which the mitochondria replaced T cells were produced. In certain embodiments, the at least one exhaustion marker is increased or decreased by at least 0.9 fold relative to the exhausted T cells from which the mitochondria replaced T cells were produced. In certain embodiments, the at least one exhaustion marker is increased or decreased by at least 1.0 fold relative to the exhausted T cells from which the mitochondria replaced T cells were produced. In certain embodiments, the at least one exhaustion marker is increased or decreased by at least 1.1 fold relative to the exhausted T cells from which the mitochondria replaced T cells were produced. In certain embodiments, the at least one exhaustion marker is increased or decreased by at least 1.15 fold relative to the exhausted T cells from which the mitochondria replaced T cells were produced. In certain embodiments, the at least one exhaustion marker is increased or decreased by at least 1.20 fold relative to the exhausted T cells from which the mitochondria replaced T cells were produced. In certain embodiments, the at least one exhaustion marker is increased or decreased by at least 1.25 fold relative to the exhausted T cells from which the mitochondria replaced T cells were produced. In certain embodiments, the at least one exhaustion marker is increased or decreased by at least 1.3 fold relative to the exhausted T cells from which the mitochondria replaced T cells were produced. In certain embodiments, the at least one exhaustion marker is increased or decreased by at least 1.4 fold relative to the exhausted T cells from which the mitochondria replaced T cells were produced. In certain embodiments, the at least one exhaustion marker is increased or decreased by at least 1.5 fold relative to the exhausted T cells from which the mitochondria replaced T cells were produced. In certain embodiments, the at least one exhaustion marker is increased or decreased by at least 1.75 fold, relative to the exhausted T cells from which the mitochondria replaced T cells were produced. In some embodiments, the at least one exhaustion marker is increased or decreased by at least 2 fold, relative to the exhausted T cells from which the mitochondria replaced T cells were produced. In certain embodiments, the at least one exhaustion marker is increased or decreased by at least 2.5 fold, relative to the exhausted T cells from which the mitochondria replaced T cells were produced. In some embodiments, the at least one exhaustion marker is increased or decreased by at least 3 fold, relative to the exhausted T cells from which the mitochondria replaced T cells were produced. In certain embodiments, the at least one exhaustion marker is increased or decreased by at least 4 fold, relative to the exhausted T cells from which the mitochondria replaced T cells were produced. In some embodiments, the at least one exhaustion marker is increased or decreased by at least 5 fold relative to the exhausted T cells from which the mitochondria replaced T cells were produced. In certain embodiments, the at least one exhaustion marker is increased or decreased by approximately 0.2 fold to approximately 1.25 fold relative to the exhausted T cells from which the mitochondria replaced T cells were produced. In certain embodiments, the at least one exhaustion marker is increased or decreased by approximately 0.2 fold to approximately 1.0 fold relative to the exhausted T cells from which the mitochondria replaced T cells were produced. In certain embodiments, the at least one exhaustion marker is increased or decreased by approximately 0.2 fold to approximately 0.8 fold relative to the exhausted T cells from which the mitochondria replaced T cells were produced. In certain embodiments, the at least one exhaustion marker is increased or decreased by approximately 0.2 fold to approximately 0.6 fold relative to the exhausted T cells from which the mitochondria replaced T cells were produced. In certain embodiments, the at least one exhaustion marker is increased or decreased by approximately 0.8 fold to approximately 1.25 fold relative to the exhausted T cells from which the mitochondria replaced T cells were produced. In certain embodiments, the at least one exhaustion marker is increased or decreased by approximately 0.5 fold to approximately 1.25 fold relative to the exhausted T cells from which the mitochondria replaced T cells were produced. In certain embodiments, the at least one exhaustion marker is increased or decreased by approximately 0.8 fold to approximately 1.0 fold relative to the exhausted T cells from which the mitochondria replaced T cells were produced. In some embodiments, the at least one exhaustion marker is increased or decreased by approximately 1.25 fold to approximately 1.50 fold, approximately 1.50 fold to approximately 2 fold, approximately 1.75 fold to approximately 2 fold, or approximately 2 fold to approximately 4 fold, relative to the exhausted T cells from which the mitochondria replaced T cells were produced. In specific embodiments, the level of two, three or more markers of T cell exhaustion are altered in mitochondria replaced T cells relative to the level of the two, three or more markers by the exhausted T cells from which the mitochondria replaced T cells were produced. In specific embodiments, the alteration in level of one, two, three or more markers of T cell exhaustion by mitochondria replaced T cells relative to the exhausted T cells from which the mitochondria replaced T cells were produced is indicative of an improvement of one, two or more effector functions of the T cells. In certain embodiments, the level of one, two, three or more markers of T cell exhaustion are altered at the RNA level relative to the exhausted T cells from which the mitochondria replaced T cells were produced. In some embodiments, the level of one, two, three or more markers of T cell exhaustion are altered at the protein level relative to the exhausted T cells from which the mitochondria replaced T cells were produced. In certain embodiments, the level of one, two, three or more markers of T cell exhaustion are altered at the RNA level and the protein level relative to the exhausted T cells from which the mitochondria replaced T cells were produced. In certain embodiments, the methods provided herein for generating a mitochondria replaced T cell are performed in vitro or ex vivo.

Accordingly, in one aspect, provided herein is a method for producing mitochondria replaced T cells from exhausted T cells, the method comprising incubating exhausted T cells having reduced endogenous mitochondria DNA (mtDNA) copy number with isolated exogenous mitochondria for a sufficient period of time to generate mitochondria replaced T cells in which the mitochondria replaced T cells have an altered (e.g., decreased or increased) level of at least one marker of T cell exhaustion (e.g., PD-1 expression or IL-2 secretion) relative to the level of the at least one marker of T cell exhaustion by the exhausted T cells from which the mitochondria replaced T cells were produced. In certain embodiments, the at least one exhaustion marker is increased or decreased by at least 5%, relative to the exhausted T cells from which the mitochondria replaced T cells were produced. In certain embodiments, the at least one exhaustion marker is increased or decreased by at least 10%, relative to the exhausted T cells from which the mitochondria replaced T cells were produced. In certain embodiments, the at least one exhaustion marker is increased or decreased by at least 15%, relative to the exhausted T cells from which the mitochondria replaced T cells were produced. In certain embodiments, the at least one exhaustion marker is increased or decreased by at least 20%, relative to the exhausted T cells from which the mitochondria replaced T cells were produced. In certain embodiments, the at least one exhaustion marker is increased or decreased by at least 25%, relative to the exhausted T cells from which the mitochondria replaced T cells were produced. In certain embodiments, the at least one exhaustion marker is increased or decreased by at least 30%, relative to the exhausted T cells from which the mitochondria replaced T cells were produced. In certain embodiments, the at least one exhaustion marker is increased or decreased by at least 35%, relative to the exhausted T cells from which the mitochondria replaced T cells were produced. In certain embodiments, the at least one exhaustion marker is increased or decreased by at least 40% relative to the exhausted T cells from which the mitochondria replaced T cells were produced. In certain embodiments, the at least one exhaustion marker is increased or decreased by approximately 45%, relative to the exhausted T cells from which the mitochondria replaced T cells were produced. In certain embodiments, the at least one exhaustion marker is increased or decreased by at least 50% relative to the exhausted T cells from which the mitochondria replaced T cells were produced. In certain embodiments, the at least one exhaustion marker is increased or decreased by at least 60% relative to the exhausted T cells from which the mitochondria replaced T cells were produced. In certain embodiments, the at least one exhaustion marker is increased or decreased by at least 70% relative to the exhausted T cells from which the mitochondria replaced T cells were produced. In certain embodiments, the at least one exhaustion marker is increased or decreased by at least 80% relative to the exhausted T cells from which the mitochondria replaced T cells were produced. In certain embodiments, the at least one exhaustion marker is increased or decreased by at least 90% relative to the exhausted T cells from which the mitochondria replaced T cells were produced. In certain embodiments, the at least one exhaustion marker is increased or decreased by at least 95% relative to the exhausted T cells from which the mitochondria replaced T cells were produced. In certain embodiments, the at least one exhaustion marker is increased by at least 100% relative to the exhausted T cells from which the mitochondria replaced T cells were produced. In certain embodiments, the at least one exhaustion marker is increased by at least 200% relative to the exhausted T cells from which the mitochondria replaced T cells were produced. In certain embodiments, the at least one exhaustion marker is increased by at least 300% relative to the exhausted T cells from which the mitochondria replaced T cells were produced. In certain embodiments, the at least one exhaustion marker is increased by at least 400% relative to the exhausted T cells from which the mitochondria replaced T cells were produced. In certain embodiments, the at least one exhaustion marker is increased by at least 500% relative to the exhausted T cells from which the mitochondria replaced T cells were produced. In some embodiments, the at least one exhaustion marker is increased or decreased by approximately 5% to approximately 50%, approximately 10% to approximately 50%, approximately 10% to approximately 40%, approximately 10% to approximately 30%, approximately 20% to approximately 50%, approximately 20% to approximately 40%, 10% to approximately 20%, approximately 20% to approximately 30%, approximately 25% to approximately 50%, or approximately 40% to approximately 60%, relative to the exhausted T cells from which the mitochondria replaced T cells were produced. In specific embodiments, the level of two, three or more markers of T cell exhaustion are altered in mitochondria replaced T cells relative to the level of the two, three or more markers by the exhausted T cells from which the mitochondria replaced T cells were produced. In specific embodiments, the alteration in level of one, two, three or more markers of T cell exhaustion by mitochondria replaced T cells relative to the exhausted T cells from which the mitochondria replaced T cells were produced is indicative of an improvement of one, two or more effector functions of the T cells. In certain embodiments, the level of one, two, three or more markers of T cell exhaustion are altered at the RNA level relative to the exhausted T cells from which the mitochondria replaced T cells were produced. In some embodiments, the level of one, two, three or more markers of T cell exhaustion are altered at the protein level relative to the exhausted T cells from which the mitochondria replaced T cells were produced. In certain embodiments, the level of one, two, three or more markers of T cell exhaustion are altered at the RNA level and the protein level relative to the exhausted T cells from which the mitochondria replaced T cells were produced. In certain embodiments, the methods provided herein for generating a mitochondria replaced T cell are performed in vitro or ex vivo.

In another aspect, provided herein is a method for producing mitochondria replaced T cells from exhausted T cells, the method comprising (a) transfecting or transforming exhausted T cells with a nucleic acid sequence comprising a nucleotide sequence encoding XbaIR (in specific embodiments, the nucleic acid sequence comprises a nucleotide sequence encoding a fusion protein comprising a mitochondrial-targeted sequence (MTS) and XbaIR) to reduce endogenous mitochondrial DNA (mtDNA) copy number; and (b) incubating the exhausted T cells having reduced mitochondria DNA (mtDNA) with isolated exogenous mitochondria for a sufficient period of time to generate mitochondria replaced T cells in which the mitochondria replaced T cells have an altered (e.g., decreased or increased) level of at least one marker of T cell exhaustion (e.g., PD-1 expression or IL-2 secretion) relative to the level of the at least one marker of T cell exhaustion by the exhausted T cells from which the mitochondria replaced T cells were produced. In certain embodiments, the at least one exhaustion marker is increased or decreased by at least 0.1 fold relative to the exhausted T cells from which the mitochondria replaced T cells were produced. In certain embodiments, the at least one exhaustion marker is increased or decreased by at least 0.2 fold relative to the exhausted T cells from which the mitochondria replaced T cells were produced. In certain embodiments, the at least one exhaustion marker is increased or decreased by at least 0.3 fold relative to the exhausted T cells from which the mitochondria replaced T cells were produced. In certain embodiments, the at least one exhaustion marker is increased or decreased by at least 0.4 fold relative to the exhausted T cells from which the mitochondria replaced T cells were produced. In certain embodiments, the at least one exhaustion marker is increased or decreased by at least 0.5 fold relative to the exhausted T cells from which the mitochondria replaced T cells were produced. In certain embodiments, the at least one exhaustion marker is increased or decreased by at least 0.6 fold relative to the exhausted T cells from which the mitochondria replaced T cells were produced. In certain embodiments, the at least one exhaustion marker is increased or decreased by at least 0.7 fold relative to the exhausted T cells from which the mitochondria replaced T cells were produced. In certain embodiments, the at least one exhaustion marker is increased or decreased by at least 0.8 fold relative to the exhausted T cells from which the mitochondria replaced T cells were produced. In certain embodiments, the at least one exhaustion marker is increased or decreased by at least 0.9 fold relative to the exhausted T cells from which the mitochondria replaced T cells were produced. In certain embodiments, the at least one exhaustion marker is increased or decreased by at least 1.0 fold relative to the exhausted T cells from which the mitochondria replaced T cells were produced. In certain embodiments, the at least one exhaustion marker is increased or decreased by at least 1.1 fold relative to the exhausted T cells from which the mitochondria replaced T cells were produced. In certain embodiments, the at least one exhaustion marker is increased or decreased by at least 1.15 fold relative to the exhausted T cells from which the mitochondria replaced T cells were produced. In certain embodiments, the at least one exhaustion marker is increased or decreased by at least 1.20 fold relative to the exhausted T cells from which the mitochondria replaced T cells were produced. In certain embodiments, the at least one exhaustion marker is increased or decreased by at least 1.25 fold relative to the exhausted T cells from which the mitochondria replaced T cells were produced. In certain embodiments, the at least one exhaustion marker is increased or decreased by at least 1.3 fold relative to the exhausted T cells from which the mitochondria replaced T cells were produced. In certain embodiments, the at least one exhaustion marker is increased or decreased by at least 1.4 fold relative to the exhausted T cells from which the mitochondria replaced T cells were produced. In certain embodiments, the at least one exhaustion marker is increased or decreased by at least 1.5 fold relative to the exhausted T cells from which the mitochondria replaced T cells were produced. In some embodiments, the at least one exhaustion marker is increased or decreased by at least 2 fold relative to the exhausted T cells from which the mitochondria replaced T cells were produced. In some embodiments, the at least one exhaustion marker is increased or decreased by at least 2.5 fold relative to the exhausted T cells from which the mitochondria replaced T cells were produced. In some embodiments, the at least one exhaustion marker is increased or decreased by at least 3 fold relative to the exhausted T cells from which the mitochondria replaced T cells were produced. In some embodiments, the at least one exhaustion marker is increased or decreased by at least 5 fold relative to the exhausted T cells from which the mitochondria replaced T cells were produced. In some embodiments, the at least one exhaustion marker is increased or decreased by at least 5 fold relative to the exhausted T cells from which the mitochondria replaced T cells were produced. In certain embodiments, the at least one exhaustion marker is increased or decreased by approximately 0.2 fold to approximately 1.25 fold relative to the exhausted T cells from which the mitochondria replaced T cells were produced. In certain embodiments, the at least one exhaustion marker is increased or decreased by approximately 0.2 fold to approximately 1.0 fold relative to the exhausted T cells from which the mitochondria replaced T cells were produced. In certain embodiments, the at least one exhaustion marker is increased or decreased by approximately 0.2 fold to approximately 0.8 fold relative to the exhausted T cells from which the mitochondria replaced T cells were produced. In certain embodiments, the at least one exhaustion marker is increased or decreased by approximately 0.2 fold to approximately 0.6 fold relative to the exhausted T cells from which the mitochondria replaced T cells were produced. In certain embodiments, the at least one exhaustion marker is increased or decreased by approximately 0.8 fold to approximately 1.25 fold relative to the exhausted T cells from which the mitochondria replaced T cells were produced. In certain embodiments, the at least one exhaustion marker is increased or decreased by approximately 0.5 fold to approximately 1.25 fold relative to the exhausted T cells from which the mitochondria replaced T cells were produced. In certain embodiments, the at least one exhaustion marker is increased or decreased by approximately 0.8 fold to approximately 1.0 fold relative to the exhausted T cells from which the mitochondria replaced T cells were produced. In some embodiments, the at least one exhaustion marker is increased or decreased by approximately 1.25 fold to approximately 1.50 fold, approximately 1.50 fold to approximately 2 fold, approximately 1.75 fold to approximately 2 fold, or approximately 2 fold to approximately 4 fold, relative to the exhausted T cells from which the mitochondria replaced T cells were produced.

In another aspect, provided herein is a method for producing mitochondria replaced T cells from exhausted T cells, the method comprising (a) transfecting or transforming exhausted T cells with a nucleic acid sequence comprising a nucleotide sequence encoding XbaIR (in specific embodiments, the nucleic acid sequence comprises a nucleotide sequence encoding a fusion protein comprising a mitochondrial-targeted sequence (MTS) and XbaIR) to reduce endogenous mitochondrial DNA (mtDNA) copy number; and (b) incubating the exhausted T cells having reduced mitochondria DNA (mtDNA) with isolated exogenous mitochondria for a sufficient period of time to generate mitochondria replaced T cells in which the mitochondria replaced T cells have an altered (e.g., decreased or increased) level of at least one marker of T cell exhaustion (e.g., PD-1 expression or IL-2 secretion) relative to the level of the at least one marker of T cell exhaustion by the exhausted T cells from which the mitochondria replaced T cells were produced. In certain embodiments, the at least one exhaustion marker is increased or decreased by at least 5%, relative to the exhausted T cells from which the mitochondria replaced T cells were produced. In certain embodiments, the at least one exhaustion marker is increased or decreased by at least 10%, relative to the exhausted T cells from which the mitochondria replaced T cells were produced. In certain embodiments, the at least one exhaustion marker is increased or decreased by at least 15%, relative to the exhausted T cells from which the mitochondria replaced T cells were produced. In certain embodiments, the at least one exhaustion marker is increased or decreased by at least 20%, relative to the exhausted T cells from which the mitochondria replaced T cells were produced. In certain embodiments, the at least one exhaustion marker is increased or decreased by at least 25%, relative to the exhausted T cells from which the mitochondria replaced T cells were produced. In certain embodiments, the at least one exhaustion marker is increased or decreased by at least 30%, relative to the exhausted T cells from which the mitochondria replaced T cells were produced. In certain embodiments, the at least one exhaustion marker is increased or decreased by at least 35%, relative to the exhausted T cells from which the mitochondria replaced T cells were produced. In certain embodiments, the at least one exhaustion marker is increased or decreased by at least 40% relative to the exhausted T cells from which the mitochondria replaced T cells were produced. In certain embodiments, the at least one exhaustion marker is increased or decreased by approximately 45%, relative to the exhausted T cells from which the mitochondria replaced T cells were produced. In certain embodiments, the at least one exhaustion marker is increased or decreased by at least 50% relative to the exhausted T cells from which the mitochondria replaced T cells were produced. In certain embodiments, the at least one exhaustion marker is increased or decreased by at least 60% relative to the exhausted T cells from which the mitochondria replaced T cells were produced. In certain embodiments, the at least one exhaustion marker is increased or decreased by at least 70% relative to the exhausted T cells from which the mitochondria replaced T cells were produced. In certain embodiments, the at least one exhaustion marker is increased or decreased by at least 80% relative to the exhausted T cells from which the mitochondria replaced T cells were produced. In certain embodiments, the at least one exhaustion marker is increased or decreased by at least 90% relative to the exhausted T cells from which the mitochondria replaced T cells were produced. In certain embodiments, the at least one exhaustion marker is increased or decreased by at least 95% relative to the exhausted T cells from which the mitochondria replaced T cells were produced. In certain embodiments, the at least one exhaustion marker is increased by at least 100% relative to the exhausted T cells from which the mitochondria replaced T cells were produced. In certain embodiments, the at least one exhaustion marker is increased by at least 200% relative to the exhausted T cells from which the mitochondria replaced T cells were produced. In certain embodiments, the at least one exhaustion marker is increased by at least 300% relative to the exhausted T cells from which the mitochondria replaced T cells were produced. In certain embodiments, the at least one exhaustion marker is increased by at least 400% relative to the exhausted T cells from which the mitochondria replaced T cells were produced. In certain embodiments, the at least one exhaustion marker is increased by at least 500% relative to the exhausted T cells from which the mitochondria replaced T cells were produced. In some embodiments, the at least one exhaustion marker is increased or decreased by approximately 5% to approximately 50%, approximately 10% to approximately 50%, approximately 10% to approximately 40%, approximately 10% to approximately 30%, approximately 20% to approximately 50%, approximately 20% to approximately 40%, 10% to approximately 20%, approximately 20% to approximately 30%, approximately 25% to approximately 50%, or approximately 40% to approximately 60%, relative to the exhausted T cells from which the mitochondria replaced T cells were produced. In specific embodiments, the level of two, three or more markers of T cell exhaustion are altered in mitochondria replaced T cells relative to the level of the two, three or more markers by the exhausted T cells from which the mitochondria replaced T cells were produced. In specific embodiments, the alteration in level of one, two, three or more markers of T cell exhaustion by mitochondria replaced T cells relative to the exhausted T cells from which the mitochondria replaced T cells were produced is indicative of an improvement of one, two or more effector functions of the T cells. In certain embodiments, the level of one, two, three or more markers of T cell exhaustion are altered at the RNA level relative to the exhausted T cells from which the mitochondria replaced T cells were produced. In some embodiments, the level of one, two, three or more markers of T cell exhaustion are altered at the protein level relative to the exhausted T cells from which the mitochondria replaced T cells were produced. In certain embodiments, the level of one, two, three or more markers of T cell exhaustion are altered at the RNA level and the protein level relative to the exhausted T cells from which the mitochondria replaced T cells were produced. In certain embodiments, the methods provided herein for generating a mitochondria replaced T cell are performed in vitro or ex vivo.

In a specific aspect, provided herein is a method for producing mitochondria replaced T cells from exhausted T cells, the method comprising (a) electroporating exhausted T cells with a nucleic acid sequence comprising a nucleotide sequence encoding XbaIR (in specific embodiments, the nucleic acid sequence comprises a nucleotide sequence encoding a fusion protein comprising a mitochondrial-targeted sequence (MTS) and XbaIR) to reduce endogenous mitochondrial DNA (mtDNA) copy number; and (b) incubating the exhausted T cells having reduced mitochondria DNA (mtDNA) with isolated exogenous mitochondria for a sufficient period of time to generate mitochondria replaced T cells in which the mitochondria replaced T cells have an altered (e.g., decreased or increased) level of at least one marker of T cell exhaustion (e.g., PD-1 expression or IL-2 secretion) relative to the level of the at least one marker of T cell exhaustion by the exhausted T cells from which the mitochondria replaced T cells were produced. In certain embodiments, the at least one exhaustion marker is increased or decreased by at least 0.1 fold relative to the exhausted T cells from which the mitochondria replaced T cells were produced. In certain embodiments, the at least one exhaustion marker is increased or decreased by at least 0.2 fold relative to the exhausted T cells from which the mitochondria replaced T cells were produced. In certain embodiments, the at least one exhaustion marker is increased or decreased by at least 0.3 fold relative to the exhausted T cells from which the mitochondria replaced T cells were produced. In certain embodiments, the at least one exhaustion marker is increased or decreased by at least 0.4 fold relative to the exhausted T cells from which the mitochondria replaced T cells were produced. In certain embodiments, the at least one exhaustion marker is increased or decreased by at least 0.5 fold relative to the exhausted T cells from which the mitochondria replaced T cells were produced. In certain embodiments, the at least one exhaustion marker is increased or decreased by at least 0.6 fold relative to the exhausted T cells from which the mitochondria replaced T cells were produced. In certain embodiments, the at least one exhaustion marker is increased or decreased by at least 0.7 fold relative to the exhausted T cells from which the mitochondria replaced T cells were produced. In certain embodiments, the at least one exhaustion marker is increased or decreased by at least 0.8 fold relative to the exhausted T cells from which the mitochondria replaced T cells were produced. In certain embodiments, the at least one exhaustion marker is increased or decreased by at least 0.9 fold relative to the exhausted T cells from which the mitochondria replaced T cells were produced. In certain embodiments, the at least one exhaustion marker is increased or decreased by at least 1.0 fold relative to the exhausted T cells from which the mitochondria replaced T cells were produced. In certain embodiments, the at least one exhaustion marker is increased or decreased by at least 1.1 fold relative to the exhausted T cells from which the mitochondria replaced T cells were produced. In certain embodiments, the at least one exhaustion marker is increased or decreased by at least 1.15 fold relative to the exhausted T cells from which the mitochondria replaced T cells were produced. In certain embodiments, the at least one exhaustion marker is increased or decreased by at least 1.20 fold relative to the exhausted T cells from which the mitochondria replaced T cells were produced. In certain embodiments, the at least one exhaustion marker is increased or decreased by at least 1.25 fold relative to the exhausted T cells from which the mitochondria replaced T cells were produced. In certain embodiments, the at least one exhaustion marker is increased or decreased by at least 1.3 fold relative to the exhausted T cells from which the mitochondria replaced T cells were produced. In certain embodiments, the at least one exhaustion marker is increased or decreased by at least 1.4 fold relative to the exhausted T cells from which the mitochondria replaced T cells were produced. In certain embodiments, the at least one exhaustion marker is increased or decreased by at least 1.5 fold relative to the exhausted T cells from which the mitochondria replaced T cells were produced. In certain embodiments, the at least one exhaustion marker is increased or decreased by at least 1.75 fold, relative to the exhausted T cells from which the mitochondria replaced T cells were produced. In certain embodiments, the at least one exhaustion marker is increased or decreased by at least 2.5 fold, relative to the exhausted T cells from which the mitochondria replaced T cells were produced. In some embodiments, the at least one exhaustion marker is increased or decreased by at least 3 fold, relative to the exhausted T cells from which the mitochondria replaced T cells were produced. In certain embodiments, the at least one exhaustion marker is increased or decreased by at least 4 fold, relative to the exhausted T cells from which the mitochondria replaced T cells were produced. In some embodiments, the at least one exhaustion marker is increased or decreased by at least 2 fold relative to the exhausted T cells from which the mitochondria replaced T cells were produced. In some embodiments, the at least one exhaustion marker is increased or decreased by at least 5 fold relative to the exhausted T cells from which the mitochondria replaced T cells were produced. In certain embodiments, the at least one exhaustion marker is increased or decreased by approximately 0.2 fold to approximately 1.25 fold relative to the exhausted T cells from which the mitochondria replaced T cells were produced. In certain embodiments, the at least one exhaustion marker is increased or decreased by approximately 0.2 fold to approximately 1.0 fold relative to the exhausted T cells from which the mitochondria replaced T cells were produced. In certain embodiments, the at least one exhaustion marker is increased or decreased by approximately 0.2 fold to approximately 0.8 fold relative to the exhausted T cells from which the mitochondria replaced T cells were produced. In certain embodiments, the at least one exhaustion marker is increased or decreased by approximately 0.2 fold to approximately 0.6 fold relative to the exhausted T cells from which the mitochondria replaced T cells were produced. In certain embodiments, the at least one exhaustion marker is increased or decreased by approximately 0.8 fold to approximately 1.25 fold relative to the exhausted T cells from which the mitochondria replaced T cells were produced. In certain embodiments, the at least one exhaustion marker is increased or decreased by approximately 0.5 fold to approximately 1.25 fold relative to the exhausted T cells from which the mitochondria replaced T cells were produced. In certain embodiments, the at least one exhaustion marker is increased or decreased by approximately 0.8 fold to approximately 1.0 fold relative to the exhausted T cells from which the mitochondria replaced T cells were produced. In some embodiments, the at least one exhaustion marker is increased or decreased by approximately 1.25 fold to approximately 1.50 fold, approximately 1.50 fold to approximately 2 fold, approximately 1.75 fold to approximately 2 fold, or approximately 2 fold to approximately 4 fold, relative to the exhausted T cells from which the mitochondria replaced T cells were produced. In specific embodiments, the level of two, three or more markers of T cell exhaustion are altered in mitochondria replaced T cells relative to the level of the two, three or more markers by the exhausted T cells from which the mitochondria replaced T cells were produced. In specific embodiments, the alteration in level of one, two, three or more markers of T cell exhaustion by mitochondria replaced T cells relative to the exhausted T cells from which the mitochondria replaced T cells were produced is indicative of an improvement of one, two or more effector functions of the T cells. In certain embodiments, the level of one, two, three or more markers of T cell exhaustion are altered at the RNA level relative to the exhausted T cells from which the mitochondria replaced T cells were produced. In some embodiments, the level of one, two, three or more markers of T cell exhaustion are altered at the protein level relative to the exhausted T cells from which the mitochondria replaced T cells were produced. In certain embodiments, the level of one, two, three or more markers of T cell exhaustion are altered at the RNA level and the protein level relative to the exhausted T cells from which the mitochondria replaced T cells were produced. In certain embodiments, the methods provided herein for generating a mitochondria replaced T cell are performed in vitro or ex vivo.

In a specific aspect, provided herein is a method for producing mitochondria replaced T cells from exhausted T cells, the method comprising (a) electroporating exhausted T cells with a nucleic acid sequence comprising a nucleotide sequence encoding XbaIR (in specific embodiments, the nucleic acid sequence comprises a nucleotide sequence encoding a fusion protein comprising a mitochondrial-targeted sequence (MTS) and XbaIR) to reduce endogenous mitochondrial DNA (mtDNA) copy number; and (b) incubating the exhausted T cells having reduced mitochondria DNA (mtDNA) with isolated exogenous mitochondria for a sufficient period of time to generate mitochondria replaced T cells in which the mitochondria replaced T cells have an altered (e.g., decreased or increased) level of at least one marker of T cell exhaustion (e.g., PD-1 expression or IL-2 secretion) relative to the level of the at least one marker of T cell exhaustion by the exhausted T cells from which the mitochondria replaced T cells were produced. In certain embodiments, the at least one exhaustion marker is increased or decreased by at least 5%, relative to the exhausted T cells from which the mitochondria replaced T cells were produced. In certain embodiments, the at least one exhaustion marker is increased or decreased by at least 10%, relative to the exhausted T cells from which the mitochondria replaced T cells were produced. In certain embodiments, the at least one exhaustion marker is increased or decreased by at least 15%, relative to the exhausted T cells from which the mitochondria replaced T cells were produced. In certain embodiments, the at least one exhaustion marker is increased or decreased by at least 20%, relative to the exhausted T cells from which the mitochondria replaced T cells were produced. In certain embodiments, the at least one exhaustion marker is increased or decreased by at least 25%, relative to the exhausted T cells from which the mitochondria replaced T cells were produced. In certain embodiments, the at least one exhaustion marker is increased or decreased by at least 30%, relative to the exhausted T cells from which the mitochondria replaced T cells were produced. In certain embodiments, the at least one exhaustion marker is increased or decreased by at least 35%, relative to the exhausted T cells from which the mitochondria replaced T cells were produced. In certain embodiments, the at least one exhaustion marker is increased or decreased by at least 40% relative to the exhausted T cells from which the mitochondria replaced T cells were produced. In certain embodiments, the at least one exhaustion marker is increased or decreased by approximately 45%, relative to the exhausted T cells from which the mitochondria replaced T cells were produced. In certain embodiments, the at least one exhaustion marker is increased or decreased by at least 50% relative to the exhausted T cells from which the mitochondria replaced T cells were produced. In certain embodiments, the at least one exhaustion marker is increased or decreased by at least 60% relative to the exhausted T cells from which the mitochondria replaced T cells were produced. In certain embodiments, the at least one exhaustion marker is increased or decreased by at least 70% relative to the exhausted T cells from which the mitochondria replaced T cells were produced. In certain embodiments, the at least one exhaustion marker is increased or decreased by at least 80% relative to the exhausted T cells from which the mitochondria replaced T cells were produced. In certain embodiments, the at least one exhaustion marker is increased or decreased by at least 90% relative to the exhausted T cells from which the mitochondria replaced T cells were produced. In certain embodiments, the at least one exhaustion marker is increased or decreased by at least 95% relative to the exhausted T cells from which the mitochondria replaced T cells were produced. In certain embodiments, the at least one exhaustion marker is increased by at least 100% relative to the exhausted T cells from which the mitochondria replaced T cells were produced. In certain embodiments, the at least one exhaustion marker is increased by at least 200% relative to the exhausted T cells from which the mitochondria replaced T cells were produced. In certain embodiments, the at least one exhaustion marker is increased by at least 300% relative to the exhausted T cells from which the mitochondria replaced T cells were produced. In certain embodiments, the at least one exhaustion marker is increased by at least 400% relative to the exhausted T cells from which the mitochondria replaced T cells were produced. In certain embodiments, the at least one exhaustion marker is increased by at least 500% relative to the exhausted T cells from which the mitochondria replaced T cells were produced. In some embodiments, the at least one exhaustion marker is increased or decreased by approximately 5% to approximately 50%, approximately 10% to approximately 50%, approximately 10% to approximately 40%, approximately 10% to approximately 30%, approximately 20% to approximately 50%, approximately 20% to approximately 40%, 10% to approximately 20%, approximately 20% to approximately 30%, approximately 25% to approximately 50%, or approximately 40% to approximately 60%, relative to the exhausted T cells from which the mitochondria replaced T cells were produced. In specific embodiments, the level of two, three or more markers of T cell exhaustion are altered in mitochondria replaced T cells relative to the level of the two, three or more markers by the exhausted T cells from which the mitochondria replaced T cells were produced. In specific embodiments, the alteration in level of one, two, three or more markers of T cell exhaustion by mitochondria replaced T cells relative to the exhausted T cells from which the mitochondria replaced T cells were produced is indicative of an improvement of one, two or more effector functions of the T cells. In certain embodiments, the level of one, two, three or more markers of T cell exhaustion are altered at the RNA level relative to the exhausted T cells from which the mitochondria replaced T cells were produced. In some embodiments, the level of one, two, three or more markers of T cell exhaustion are altered at the protein level relative to the exhausted T cells from which the mitochondria replaced T cells were produced. In certain embodiments, the level of one, two, three or more markers of T cell exhaustion are altered at the RNA level and the protein level relative to the exhausted T cells from which the mitochondria replaced T cells were produced. In certain embodiments, the methods provided herein for generating a mitochondria replaced T cell are performed in vitro or ex vivo.

Various markers of T cell exhaustion are known in the art. Non-limiting examples of T cell exhaustion markers include elevated expression of a cell-surface molecule that inhibits the immune response, termed a “immune checkpoint receptor” [e.g., programmed cell death-1 (PD-1), T-cell immunoglobulin and mucin domain-containing protein 3 (TIM3), lymphocyte-activated gene-3 (LAG3), T Cell immunoglobulin and ITIM domain (TIGIT), CD160, cytotoxic T lymphocyte-associated protein 4 (CTLA-4), 2B4/CD244/SLAMF4], decreased production of chemokines (e.g., TNF-alpha, interferon-gamma, cc (beta)), and decreased cytokine production (e.g., IL-2), relative to a functional T cell. Assays for determining T cell function are known in the art, and include assays such as those described in Section 6.3.

In some embodiments, an exhausted T cell comprises an increased level of a T cell exhaustion marker, relative to a functional T cell. In certain embodiments, an exhausted T cell comprises increased expression of an immune checkpoint receptor selected from the group consisting of PD-1, TIM3, LAG3, TIGIT, CD150, CTLA-4, or 2B4/CD244/SLAMF4, relative to a functional T cell. In some embodiments, an exhausted T cell comprises increased expression of PD-1, relative to a functional T cell. In some embodiments, an exhausted T cell comprises increased expression of TIM3, relative to a functional T cell. In some embodiments, an exhausted T cell comprises increased expression of LAG3, relative to a functional T cell. In some embodiments, an exhausted T cell comprises increased expression of TIGIT, relative to a functional T cell. In some embodiments, an exhausted T cell comprises increased expression of CD150, relative to a functional T cell. In some embodiments, an exhausted T cell comprises increased expression of CTLA-4, relative to a functional T cell. In one embodiment, an exhausted T cell comprises increased expression of 2B4/CD244/SLAMF4, relative to a functional T cell.

In some embodiments, an exhausted T cell comprises increased expression of a transcription factor selected from the group consisting of TOX, TOX2, and NR4A, relative to a functional T cell. In some embodiments, an exhausted T cell comprises increased expression of TOX, relative to a functional T cell. In some embodiments, an exhausted T cell comprises increased expression of TOX2, relative to a functional T cell. In some embodiments, an exhausted T cell comprises increased expression of NR4A, relative to a functional T cell.

In some embodiments, an exhausted T cell comprises a decreased level of a T cell exhaustion marker, relative to a functional T cell. In certain embodiments, an exhausted T cell comprises decreased chemokine production (e.g., TNF-alpha, interferon-gamma, CC (β-chemokine)), and/or decreased cytokine production (e.g., IL-2), relative to a functional T cell. In some embodiments, an exhausted T cell comprises decreased production of TNF-α, relative to a functional T cell. In some embodiments, an exhausted T cell comprises decreased production of IFN-γ, relative to a functional T cell. In some embodiments, an exhausted T cell comprises decreased production of a CC chemokine, relative to a functional T cell. In some embodiments, an exhausted T cell comprises decreased production of IL-2, relative to a functional T cell.

Accordingly, in one embodiment, the exhausted T cells of the present disclosure have increased expression of an immune checkpoint receptor, and the mitochondria replaced T cells produced according to the methods provided herein from exhausted T cells have decreased expression of the immune checkpoint receptor, relative to the exhausted T cells. In certain embodiments, the mitochondria replaced T cells have decreased expression of at least PD-1, relative to the exhausted T cell. In specific embodiments, PD-1 expression in the mitochondria replaced T cells is decreased by at least 10%, relative to the exhausted T cell. In specific embodiments, PD-1 expression in the mitochondria replaced T cells is decreased by at least 20%, relative to the exhausted T cell. In specific embodiments, PD-1 expression in the mitochondria replaced T cells is decreased by at least 30%, relative to the exhausted T cell. In specific embodiments, PD-1 expression in the mitochondria replaced T cells is decreased by at least 40%, relative to the exhausted T cell. In specific embodiments, PD-1 expression in the mitochondria replaced T cells is decreased by at least 50%, relative to the exhausted T cell. In specific embodiments, PD-1 expression in the mitochondria replaced T cells is decreased by at least 60%, relative to the exhausted T cell. In specific embodiments, PD-1 expression in the mitochondria replaced T cells is decreased by at least 70%, relative to the exhausted T cell. In specific embodiments, PD-1 expression in the mitochondria replaced T cells is decreased by at least 80%, relative to the exhausted T cell. In specific embodiments, PD-1 expression in the mitochondria replaced T cells is decreased by at least 90%, relative to the exhausted T cell. In specific embodiments, PD-1 expression in the mitochondria replaced T cells is decreased by at least 95%, relative to the exhausted T cell. In some embodiments, PD-1 expression in the mitochondria replaced T cells is decreased by approximately 5% to approximately 50%, approximately 10% to approximately 50%, approximately 10% to approximately 40%, approximately 10% to approximately 30%, approximately 20% to approximately 50%, approximately 20% to approximately 40%, approximately 10% to approximately 20%, approximately 20% to approximately 30%, approximately 25% to approximately 50%, or approximately 40% to approximately 60%, relative to the exhausted T cells from which the mitochondria replaced T cells were produced.

In specific embodiments, PD-1 expression in the mitochondria replaced T cells is decreased by at least 1.1 fold, relative to the exhausted T cell. In specific embodiments, PD-1 expression in the mitochondria replaced T cells is decreased by at least 1.2 fold, relative to the exhausted T cell. In specific embodiments, PD-1 expression in the mitochondria replaced T cells is decreased by at least 1.25 fold, relative to the exhausted T cell. In specific embodiments, PD-1 expression in the mitochondria replaced T cells is decreased by at least 1.3 fold, relative to the exhausted T cells. In specific embodiments, PD-1 expression in the mitochondria replaced T cells is decreased by at least 1.4 fold, relative to the exhausted T cells. In specific embodiments, PD-1 expression in the mitochondria replaced T cells is decreased by at least 1.5 fold, relative to the exhausted T cells. In specific embodiments, PD-1 expression in the mitochondria replaced T cells is decreased by at least 1.6 fold, relative to the exhausted T cells. In specific embodiments, PD-1 expression in the mitochondria replaced T cells is decreased by at least 1.7 fold, relative to the exhausted T cells. In specific embodiments, PD-1 expression in the mitochondria replaced T cells is decreased by at least 1.8 fold, relative to the exhausted T cell. In specific embodiments, PD-1 expression in the mitochondria replaced T cells is decreased by at least 1.9 fold, relative to the exhausted T cells. In specific embodiments, PD-1 expression in the mitochondria replaced T cells is decreased by at least 2 fold, relative to the exhausted T cell. In specific embodiments, PD-1 expression in the mitochondria replaced T cells is decreased by at least 3 fold, relative to the exhausted T cells. In specific embodiments, PD-1 expression in the mitochondria replaced T cells is decreased by at least 4 fold, relative to the exhausted T cells. In specific embodiments, PD-1 expression in the mitochondria replaced T cells is decreased by at least 5 fold, relative to the exhausted T cells. In specific embodiments, PD-1 expression in the mitochondria replaced T cells is decreased by more than 5 fold, relative to the exhausted T cells. In certain embodiments, PD-1 expression in the mitochondria replaced T cells is decreased by approximately 0.2 fold to approximately 1.25 fold relative to the exhausted T cells from which the mitochondria replaced T cells were produced. In certain embodiments, PD-1 expression in the mitochondria replaced T cells is decreased by approximately 0.2 fold to approximately 1.0 fold relative to the exhausted T cells from which the mitochondria replaced T cells were produced. In certain embodiments, PD-1 expression in the mitochondria replaced T cells is decreased by approximately 0.2 fold to approximately 0.8 fold relative to the exhausted T cells from which the mitochondria replaced T cells were produced. In certain embodiments, PD-1 expression in the mitochondria replaced T cells is decreased by approximately 0.2 fold to approximately 0.6 fold relative to the exhausted T cells from which the mitochondria replaced T cells were produced. In certain embodiments, PD-1 expression in the mitochondria replaced T cells is decreased by approximately 0.8 fold to approximately 1.25 fold relative to the exhausted T cells from which the mitochondria replaced T cells were produced. In certain embodiments, PD-1 expression in the mitochondria replaced T cells is decreased by approximately 0.5 fold to approximately 1.25 fold relative to the exhausted T cells from which the mitochondria replaced T cells were produced. In certain embodiments, PD-1 expression in the mitochondria replaced T cells is decreased by approximately 0.8 fold to approximately 1.0 fold relative to the exhausted T cells from which the mitochondria replaced T cells were produced. In some embodiments, PD-1 expression in the mitochondria replaced T cells is decreased by approximately 1.25 fold to approximately 1.50 fold, approximately 1.50 fold to approximately 2 fold, approximately 1.75 fold to approximately 2 fold, or approximately 2 fold to approximately 4 fold, relative to the exhausted T cells from which the mitochondria replaced T cells were produced.

In a specific embodiment, provided herein is a method for producing mitochondria replaced T cells from exhausted T cells, the method comprising incubating exhausted T cells having reduced endogenous mitochondria DNA (mtDNA) copy number with isolated exogenous mitochondria for a sufficient period of time to generate mitochondria replaced T cells in which the expression of programmed cell death-1 (PD-1) is decreased by at least 1.1 fold relative to the expression of PD-1 by the exhausted T cells from which the mitochondria replaced T cells were produced. In certain embodiments, the PD-1 expression is decreased by at least 1.2 fold relative to the expression of PD-1 by the exhausted T cells from which the mitochondria replaced T cells were produced. In certain embodiments, the PD-1 expression is decreased by at least 1.25 fold relative to the expression of PD-1 by the exhausted T cells from which the mitochondria replaced T cells were produced. In some embodiments, the PD-1 expression is decreased by at least 1.50 fold, relative to the expression of PD-1 by the exhausted T cells from which the mitochondria replaced T cells were produced. In certain embodiments, the PD-1 expression is decreased by at least 1.75 fold, relative to the expression of PD-1 by the exhausted T cells from which the mitochondria replaced T cells were produced. In some embodiments, the PD-1 is decreased by at least 2 fold, relative to the expression of PD-1 by the exhausted T cells from which the mitochondria replaced T cells were produced. In certain embodiments, the PD-1 expression is decreased by at least 2.5 fold, relative to the expression of PD-1 by the exhausted T cells from which the mitochondria replaced T cells were produced. In some embodiments, the PD-1 expression is decreased by at least 3 fold, relative to the expression of PD-1 by the exhausted T cells from which the mitochondria replaced T cells were produced. In certain embodiments, the PD-1 expression is decreased by at least 4 fold. In certain embodiments, the PD-1 expression is decreased by approximately 1.1 fold to approximately 1.25 fold relative to the expression of PD-1 by the exhausted T cells from which the mitochondria replaced T cells were produced. In some embodiments, the PD-1 expression is decreased by approximately 1.25 fold to approximately 1.50 fold, approximately 1.50 fold to approximately 2 fold, approximately 1.75 fold to approximately 2 fold, or approximately 2 fold to approximately 4 fold, relative to the expression of PD-1 by the exhausted T cells from which the mitochondria replaced T cells were produced. In some embodiments, the PD-1 expression is decreased by approximately 1.25 fold to approximately 1.50 fold, approximately 1.50 fold to approximately 2 fold, approximately 1.75 fold to approximately 2 fold, or approximately 2 fold to approximately 4 fold, relative to the expression of PD-1 by the exhausted T cells from which the mitochondria replaced T cells were produced. In specific embodiments, the expression of PD-1 and one, two, three or more other markers of T cell exhaustion are reduced in mitochondria replaced T cells relative to the expression of PD-1 and the one, two, three or more other T cell exhaustion markers by the exhausted T cells from which the mitochondria replaced T cells were produced. In specific embodiments, the decrease in expression of one, two, three or more markers of T cell exhaustion by mitochondria replaced T cells relative to the expression of the same one, two, three, or more markers by the exhausted T cells from which the mitochondria replaced T cells were produced is indicative of an improvement of one, two or more effector functions of the T cells. In certain embodiments, the expression of PD-1 and, in some embodiments, the expression of one, two, three or more other markers of T cell exhaustion, are decreased at the RNA level relative to the expression of the corresponding marker by the exhausted T cells from which the mitochondria replaced T cells were produced. In some embodiments, the expression of PD-1 and, in some embodiments, the expression of one, two, three or more other markers of T cell exhaustion, are decreased at the protein level, relative to the expression of the same maker from which the mitochondria replaced T cells were produced. In certain embodiments, the expression of PD-1 and, in some embodiments, the expression of one, two, three or more other markers of T cell exhaustion, are decreased at the RNA level and the protein level, relative to the expression of the corresponding marker by the exhausted T cells from which the mitochondria replaced T cells were produced. In some embodiments, the nucleic acid sequence is RNA (e.g., mRNA). In some embodiments, the RNA is unmodified RNA. In some embodiments, the RNA is modified RNA. In some embodiments, the nucleic acid sequence is DNA (e.g., cDNA). In some embodiments, the DNA is unmodified DNA. In some embodiments, the DNA is modified DNA. Non-limiting examples of modified RNA or modified DNA include tritylated bases and unusual bases such as inosine.

In a specific embodiment, provided herein is a method for producing mitochondria replaced T cells from exhausted T cells, the method comprising incubating exhausted T cells having reduced endogenous mitochondria DNA (mtDNA) copy number with isolated exogenous mitochondria for a sufficient period of time to generate mitochondria replaced T cells in which the expression of programmed cell death-1 (PD-1) is decreased by at least 5% relative to the expression of PD-1 by the exhausted T cells from which the mitochondria replaced T cells were produced. In certain embodiments, the PD-1 expression is decreased by at least 10% relative to the expression of PD-1 by the exhausted T cells from which the mitochondria replaced T cells were produced. In certain embodiments, the PD-1 expression is decreased by at least 15% relative to the expression of PD-1 by the exhausted T cells from which the mitochondria replaced T cells were produced. In certain embodiments, the PD-1 expression is decreased by at least 20% relative to the expression of PD-1 by the exhausted T cells from which the mitochondria replaced T cells were produced. In certain embodiments, the PD-1 expression is decreased by at least 25% relative to the expression of PD-1 by the exhausted T cells from which the mitochondria replaced T cells were produced. In certain embodiments, the PD-1 expression is decreased by at least 30% relative to the expression of PD-1 by the exhausted T cells from which the mitochondria replaced T cells were produced. In certain embodiments, the PD-1 expression is decreased by at least 35% relative to the expression of PD-1 by the exhausted T cells from which the mitochondria replaced T cells were produced. In certain embodiments, the PD-1 expression is decreased by at least 40% relative to the expression of PD-1 by the exhausted T cells from which the mitochondria replaced T cells were produced. In certain embodiments, the PD-1 expression is decreased by at least 45% relative to the expression of PD-1 by the exhausted T cells from which the mitochondria replaced T cells were produced. In certain embodiments, the PD-1 expression is decreased by at least 50% relative to the expression of PD-1 by the exhausted T cells from which the mitochondria replaced T cells were produced. In certain embodiments, the PD-1 expression is decreased by at least 60% relative to the expression of PD-1 by the exhausted T cells from which the mitochondria replaced T cells were produced. In certain embodiments, the PD-1 expression is decreased by at least 70% relative to the expression of PD-1 by the exhausted T cells from which the mitochondria replaced T cells were produced. In certain embodiments, the PD-1 expression is decreased by at least 80% relative to the expression of PD-1 by the exhausted T cells from which the mitochondria replaced T cells were produced. In certain embodiments, the PD-1 expression is decreased by at least 90% relative to the expression of PD-1 by the exhausted T cells from which the mitochondria replaced T cells were produced. In certain embodiments, the PD-1 expression is decreased by at least 95% relative to the expression of PD-1 by the exhausted T cells from which the mitochondria replaced T cells were produced. In some embodiments, the PD-1 expression is decreased by approximately 5% to approximately 50%, approximately 10% to approximately 50%, approximately 10% to approximately 40%, approximately 10% to approximately 30%, approximately 20% to approximately 50%, approximately 20% to approximately 40%, approximately 10% to approximately 20%, approximately 20% to approximately 30%, approximately 25% to approximately 50%, or approximately 40% to approximately 60%, relative to the expression of PD-1 by the exhausted T cells from which the mitochondria replaced T cells were produced. In specific embodiments, the expression of PD-1 and one, two, three or more other markers of T cell exhaustion are reduced in mitochondria replaced T cells relative to the expression of PD-1 and the one, two, three or more other T cell exhaustion markers by the exhausted T cells from which the mitochondria replaced T cells were produced. In specific embodiments, the decrease in expression of one, two, three or more markers of T cell exhaustion by mitochondria replaced T cells relative to the expression of the same one, two, three, or more markers by the exhausted T cells from which the mitochondria replaced T cells were produced is indicative of an improvement of one, two or more effector functions of the T cells. In certain embodiments, the expression of PD-1 and, in some embodiments, the expression of one, two, three or more other markers of T cell exhaustion, are decreased at the RNA level relative to the expression of the corresponding marker by the exhausted T cells from which the mitochondria replaced T cells were produced. In some embodiments, the expression of PD-1 and, in some embodiments, the expression of one, two, three or more other markers of T cell exhaustion, are decreased at the protein level, relative to the expression of the same maker from which the mitochondria replaced T cells were produced. In certain embodiments, the expression of PD-1 and, in some embodiments, the expression of one, two, three or more other markers of T cell exhaustion, are decreased at the RNA level and the protein level, relative to the expression of the corresponding marker by the exhausted T cells from which the mitochondria replaced T cells were produced. In some embodiments, the nucleic acid sequence is RNA (e.g., mRNA). In some embodiments, the RNA is unmodified RNA. In some embodiments, the RNA is modified RNA. In some embodiments, the nucleic acid sequence is DNA (e.g., cDNA). In some embodiments, the DNA is unmodified DNA. In some embodiments, the DNA is modified DNA. Non-limiting examples of modified RNA or modified DNA include tritylated bases and unusual bases such as inosine.

In specific embodiments, the expression of PD-1 and one, two, three or more other markers of T cell exhaustion are reduced in mitochondria replaced T cells relative to the expression of the corresponding marker in the exhausted T cells from which they were produced. In specific embodiments, the decrease in expression of one, two, three or more markers of T cell exhaustion by mitochondria replaced T cells relative to the expression of the corresponding marker in the exhausted T cells from which they were produced is indicative of an improvement of one, two or more effector functions of the T cells. In certain embodiments, the expression of PD-1 and, in some embodiments, the expression of one, two, three or more other markers of T cell exhaustion, are decreased at the RNA level, relative to the expression of the corresponding marker in the exhausted T cells from which they were produced. In some embodiments, the expression of PD-1 and, in some embodiments, the expression of one, two, three or more other markers of T cell exhaustion, are decreased at the protein level, relative to the expression of the corresponding marker in the exhausted T cells from which they were produced. In certain embodiments, the expression of one PD-1 and, in some embodiments, the expression of one, two, three or more other markers of T cell exhaustion, are decreased at the RNA level and the protein level, relative to the expression of the corresponding marker in the exhausted T cells from which they were produced.

In certain embodiments, the expression of PD-1 is decreased by at least 1.1 fold, at least 1.25 fold, at least 1.5 fold, at least 2.0 fold, at least 2.5 fold, at least 3.0 fold, or at least 4.0 fold in the mitochondria replaced T cells and expression of at least one additional immune checkpoint receptor is also decreased relative to the expression of the corresponding marker by the exhausted T cells. In certain embodiments, the expression of PD-1 is decreased by approximately 1.1 fold to approximately 1.25 fold, 1.25 fold to approximately 1.50 fold, approximately 1.50 fold to approximately 2 fold, approximately 1.75 fold to approximately 2 fold, or approximately 2 fold to approximately 4 fold in the mitochondria replaced T cells and expression of at least one additional immune checkpoint receptor is also decreased relative to the expression of the corresponding marker by the exhausted T cells. In certain embodiments, the expression of PD-1 and the expression of at least one additional immune checkpoint receptor are decreased at the RNA level relative to the expression of the corresponding marker by the exhausted T cells. In some embodiments, the expression of PD-1 and the expression of at least one additional immune checkpoint receptor are decreased at the protein level relative to the expression of the corresponding marker by the exhausted T cells. In certain embodiments, the expression of PD-1 and the expression of at least one additional immune checkpoint receptor are decreased at the RNA level and the protein level relative to the expression of the corresponding marker by the exhausted T cells.

In certain embodiments, the expression of PD-1 is decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells and expression of at least one additional immune checkpoint receptor is also decreased relative to the expression of the corresponding marker by the exhausted T cells. In certain embodiments, the expression of PD-1 is decreased by approximately 5% to approximately 50%, approximately 10% to approximately 50%, approximately 10% to approximately 40%, approximately 10% to approximately 30%, approximately 20% to approximately 50%, approximately 20% to approximately 40%, approximately 10% to approximately 20%, approximately 20% to approximately 30%, approximately 25% to approximately 50%, or approximately 40% to approximately 60% in the mitochondria replaced T cells and expression of at least one additional immune checkpoint receptor is also decreased relative to the expression of the corresponding marker by the exhausted T cells. In certain embodiments, the expression of PD-1 and the expression of at least one additional immune checkpoint receptor are decreased at the RNA level relative to the expression of the corresponding marker by the exhausted T cells. In some embodiments, the expression of PD-1 and the expression of at least one additional immune checkpoint receptor are decreased at the protein level relative to the expression of the corresponding marker by the exhausted T cells. In certain embodiments, the expression of PD-1 and the expression of at least one additional immune checkpoint receptor are decreased at the RNA level and the protein level relative to the expression of the corresponding marker by the exhausted T cells.

In certain embodiments, the expression of PD-1 is decreased by at least 1.1 fold, at least 1.25 fold, at least 1.5 fold, at least 2.0 fold, at least 2.5 fold, at least 3.0 fold, or at least 4.0 fold in the mitochondria replaced T cells, and the expression of the at least one immune checkpoint receptor selected from the group consisting of TIM3, LAG3, TIGIT, CD160, CTLA-4, and 2B4/CD244/SLAMF4, is also decreased relative to the expression of the corresponding marker by the exhausted T cells. In certain embodiments, the expression of PD-1 is decreased by approximately 1.1 fold to approximately 1.25 fold, 1.25 fold to approximately 1.50 fold, approximately 1.50 fold to approximately 2 fold, approximately 1.75 fold to approximately 2 fold, or approximately 2 fold to approximately 4 fold in the mitochondria replaced T cells, and the expression of the at least one immune checkpoint receptor selected from the group consisting of TIM3, LAG3, TIGIT, CD160, CTLA-4, and 2B4/CD244/SLAMF4, is also decreased relative to the expression of the corresponding marker by the exhausted T cells. In certain embodiments, the expression of PD-1 and the expression of at least one additional immune checkpoint receptor are decreased at the RNA level relative to the expression of the corresponding marker by the exhausted T cells. In some embodiments, the expression of PD-1 and the expression of at least one additional immune checkpoint receptor are decreased at the protein level relative to the expression of the corresponding marker by the exhausted T cells. In certain embodiments, the expression of PD-1 and the expression of at least one additional immune checkpoint receptor are decreased at the RNA level and the protein level relative to the expression of the corresponding marker by the exhausted T cells.

In certain embodiments, the expression of PD-1 is decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, and the expression of the at least one immune checkpoint receptor selected from the group consisting of TIM3, LAG3, TIGIT, CD160, CTLA-4, and 2B4/CD244/SLAMF4, is also decreased relative to the expression of the corresponding marker by the exhausted T cells. In certain embodiments, the expression of PD-1 is approximately 5% to approximately 50%, approximately 10% to approximately 50%, approximately 10% to approximately 40%, approximately 10% to approximately 30%, approximately 20% to approximately 50%, approximately 20% to approximately 40%, approximately 10% to approximately 20%, approximately 20% to approximately 30%, approximately 25% to approximately 50%, or approximately 40% to approximately 60% in the mitochondria replaced T cells, and the expression of the at least one immune checkpoint receptor selected from the group consisting of TIM3, LAG3, TIGIT, CD160, CTLA-4, and 2B4/CD244/SLAMF4, is also decreased relative to the expression of the corresponding marker by the exhausted T cells. In certain embodiments, the expression of PD-1 and the expression of at least one additional immune checkpoint receptor are decreased at the RNA level relative to the expression of the corresponding marker by the exhausted T cells. In some embodiments, the expression of PD-1 and the expression of at least one additional immune checkpoint receptor are decreased at the protein level relative to the expression of the corresponding marker by the exhausted T cells. In certain embodiments, the expression of PD-1 and the expression of at least one additional immune checkpoint receptor are decreased at the RNA level and the protein level relative to the expression of the corresponding marker by the exhausted T cells.

In certain embodiments, the expression of PD-1 is decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, and the expression of the at least one immune checkpoint receptor selected from the group consisting of TIM3, LAG3, TIGIT, CD160, CTLA-4, and 2B4/CD244/SLAMF4, is also decreased relative to the expression of the corresponding marker by the exhausted T cells. In certain embodiments, the expression of PD-1 is approximately 5% to approximately 50%, approximately 10% to approximately 50%, approximately 10% to approximately 40%, approximately 10% to approximately 30%, approximately 20% to approximately 50%, approximately 20% to approximately 40%, approximately 10% to approximately 20%, approximately 20% to approximately 30%, approximately 25% to approximately 50%, or approximately 40% to approximately 60% in the mitochondria replaced T cells, and the expression of the at least one immune checkpoint receptor selected from the group consisting of TIM3, LAG3, TIGIT, CD160, CTLA-4, and 2B4/CD244/SLAMF4, is also decreased relative to the expression of the corresponding marker by the exhausted T cells. In certain embodiments, the expression of PD-1 and the expression of at least one additional immune checkpoint receptor are decreased at the RNA level relative to the expression of the corresponding marker by the exhausted T cells. In some embodiments, the expression of PD-1 and the expression of at least one additional immune checkpoint receptor are decreased at the protein level relative to the expression of the corresponding marker by the exhausted T cells. In certain embodiments, the expression of PD-1 and the expression of at least one additional immune checkpoint receptor are decreased at the RNA level and the protein level relative to the expression of the corresponding marker by the exhausted T cells.

In certain embodiments, the expression of PD-1 and LAG3 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. In certain embodiments, the expression of PD-1 and TIGIT are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. In certain embodiments, the expression of PD-1 and CD160 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. In certain embodiments, the expression of PD-1 and CTLA-4 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. In certain embodiments, the expression of PD-1 and 2B4/CD244/SLAMF4 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. In certain embodiments, the expression is decreased at the RNA level relative to the expression of the corresponding marker by the exhausted T cells. In some embodiments, the expression is decreased at the protein level relative to the expression of the corresponding marker by the exhausted T cells. In certain embodiments, the expression is decreased at the RNA level and the protein level relative to the expression of the corresponding marker by the exhausted T cells.

In certain embodiments, the expression of PD-1 and LAG3 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. In certain embodiments, the expression of PD-1 and TIGIT are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. In certain embodiments, the expression of PD-1 and CD160 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. In certain embodiments, the expression of PD-1 and CTLA-4 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. In certain embodiments, the expression of PD-1 and 2B4/CD244/SLAMF4 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. In certain embodiments, the expression is decreased at the RNA level relative to the expression of the corresponding marker by the exhausted T cells. In some embodiments, the expression is decreased at the protein level relative to the expression of the corresponding marker by the exhausted T cells. In certain embodiments, the expression is decreased at the RNA level and the protein level relative to the expression of the corresponding marker by the exhausted T cells.

In certain embodiments, the expression of PD-1 is decreased by at least 1.1 fold, at least 1.25 fold, at least 1.5 fold, at least 2.0 fold, at least 2.5 fold, at least 3.0 fold, or at least 4.0 fold in the mitochondria replaced T cells and expression of at least two additional immune checkpoint receptors are also decreased relative to the expression of the corresponding marker by the exhausted T cells. By way of example, in some embodiments, the expression of PD-1, LAG3 and 2B4/CD244/SLAMF4 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. In some embodiments, the expression of PD-1, TIGIT, and CD160 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. In some embodiments, the expression of PD-1, CTLA-4, and LAG3 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. In certain embodiments, the expression of PD-1 and the expression of at least two additional immune checkpoint receptors are decreased at the RNA level relative to the expression of the corresponding marker by the exhausted T cells. In some embodiments, the expression of PD-1 and the expression of at least two additional immune checkpoint receptors are decreased at the protein level relative to the expression of the corresponding marker by the exhausted T cells. In certain embodiments, the expression of PD-1 and the expression of at least two additional immune checkpoint receptors are decreased at the RNA level and the protein level relative to the expression of the corresponding marker by the exhausted T cells.

In certain embodiments, the expression of PD-1 is decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells and expression of at least two additional immune checkpoint receptors are also decreased relative to the expression of the corresponding marker by the exhausted T cells. By way of example, in some embodiments, the expression of PD-1, LAG3 and 2B4/CD244/SLAMF4 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. In some embodiments, the expression of PD-1, TIGIT, and CD160 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. In some embodiments, the expression of PD-1, CTLA-4, and LAG3 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. In certain embodiments, the expression of PD-1 and the expression of at least two additional immune checkpoint receptors are decreased at the RNA level relative to the expression of the corresponding marker by the exhausted T cells. In some embodiments, the expression of PD-1 and the expression of at least two additional immune checkpoint receptors are decreased at the protein level relative to the expression of the corresponding marker by the exhausted T cells. In certain embodiments, the expression of PD-1 and the expression of at least two additional immune checkpoint receptors are decreased at the RNA level and the protein level relative to the expression of the corresponding marker by the exhausted T cells.

In certain embodiments, the expression of PD-1 is decreased by at least 1.1 fold, at least 1.25 fold, at least 1.5 fold, at least 2.0 fold, at least 2.5 fold, at least 3.0 fold, or at least 4.0 fold in the mitochondria replaced T cells and expression of at least three additional immune checkpoint receptors are also decreased relative to the expression of the corresponding marker by the exhausted T cells. By way of example, in some embodiments, the expression of PD-1, LAG3, TIGIT and 2B4/CD244/SLAMF4 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. In some embodiments, the expression of PD-1, CTLA-4, TIGIT, and CD160 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. In some embodiments, the expression of PD-1, CTLA-4, TIGIT and LAG3 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. In certain embodiments, the expression of PD-1 and the expression of at least three additional immune checkpoint receptors are decreased at the RNA level relative to the expression of the corresponding marker by the exhausted T cells. In some embodiments, the expression of PD-1 and the expression of at least three additional immune checkpoint receptors are decreased at the protein level relative to the expression of the corresponding marker by the exhausted T cells. In certain embodiments, the expression of PD-1 and the expression of at least three additional immune checkpoint receptors are decreased at the RNA level and the protein level relative to the expression of the corresponding marker by the exhausted T cells.

In certain embodiments, the expression of PD-1 is decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells and expression of at least three additional immune checkpoint receptors are also decreased relative to the expression of the corresponding marker by the exhausted T cells. By way of example, in some embodiments, the expression of PD-1, LAG3, TIGIT and 2B4/CD244/SLAMF4 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. In some embodiments, the expression of PD-1, CTLA-4, TIGIT, and CD160 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. In some embodiments, the expression of PD-1, CTLA-4, TIGIT and LAG3 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. In certain embodiments, the expression of PD-1 and the expression of at least three additional immune checkpoint receptors are decreased at the RNA level relative to the expression of the corresponding marker by the exhausted T cells. In some embodiments, the expression of PD-1 and the expression of at least three additional immune checkpoint receptors are decreased at the protein level relative to the expression of the corresponding marker by the exhausted T cells. In certain embodiments, the expression of PD-1 and the expression of at least three additional immune checkpoint receptors are decreased at the RNA level and the protein level relative to the expression of the corresponding marker by the exhausted T cells.

In certain embodiments, the expression of PD-1 is decreased by at least 1.1 fold, at least 1.25 fold, at least 1.5 fold, at least 2.0 fold, at least 2.5 fold, at least 3.0 fold, or at least 4.0 fold in the mitochondria replaced T cells and expression of at least four additional immune checkpoint receptors are also decreased relative to the expression of the corresponding marker by the exhausted T cells. By way of example, in some embodiments, the expression of PD-1, LAG3, TIGIT, CD160 and 2B4/CD244/SLAMF4 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. In some embodiments, the expression of PD-1, CTLA-4, TIGIT, LAG3 and CD160 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. In some embodiments, the expression of PD-1, CTLA-4, TIGIT, TIM3, and LAG3 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. In certain embodiments, the expression of PD-1 and the expression of at least four additional immune checkpoint receptors are decreased at the RNA level relative to the expression of the corresponding marker by the exhausted T cells. In some embodiments, the expression of PD-1 and the expression of at least four additional immune checkpoint receptors are decreased at the protein level relative to the expression of the corresponding marker by the exhausted T cells. In certain embodiments, the expression of PD-1 and the expression of at least four additional immune checkpoint receptors are decreased at the RNA level and the protein level relative to the expression of the corresponding marker by the exhausted T cells.

In certain embodiments, the expression of PD-1 is decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells and expression of at least four additional immune checkpoint receptors are also decreased relative to the expression of the corresponding marker by the exhausted T cells. By way of example, in some embodiments, the expression of PD-1, LAG3, TIGIT, CD160 and 2B4/CD244/SLAMF4 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. In some embodiments, the expression of PD-1, CTLA-4, TIGIT, LAG3 and CD160 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. In some embodiments, the expression of PD-1, CTLA-4, TIGIT, TIM3, and LAG3 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. In certain embodiments, the expression of PD-1 and the expression of at least four additional immune checkpoint receptors are decreased at the RNA level relative to the expression of the corresponding marker by the exhausted T cells. In some embodiments, the expression of PD-1 and the expression of at least four additional immune checkpoint receptors are decreased at the protein level relative to the expression of the corresponding marker by the exhausted T cells. In certain embodiments, the expression of PD-1 and the expression of at least four additional immune checkpoint receptors are decreased at the RNA level and the protein level relative to the expression of the corresponding marker by the exhausted T cells.

In certain embodiments, the expression of PD-1 is decreased by at least 1.1 fold, at least 1.25 fold, at least 1.5 fold, at least 2.0 fold, at least 2.5 fold, at least 3.0 fold, or at least 4.0 fold in the mitochondria replaced T cells and expression of at least five additional immune checkpoint receptors are also decreased relative to the expression of the corresponding marker by the exhausted T cells. By way of example, in some embodiments, the expression of PD-1, LAG3, TIGIT, CD160, CTLA-4, and 2B4/CD244/SLAMF4 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. In some embodiments, the expression of PD-1, TIM3, TIGIT, CD160, CTLA-4, and 2B4/CD244/SLAMF4 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. In some embodiments, the expression of PD-1, TIM3, LAG3, CD160, CTLA-4, and 2B4/CD244/SLAMF4 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. By way of example, in some embodiments, the expression of PD-1, LAG3, TIGIT, CD160, CTLA-4, and 2B4/CD244/SLAMF4 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells By way of example, in some embodiments, the expression of PD-1, TIM3, LAG3, TIGIT, CTLA-4, and 2B4/CD244/SLAMF4 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. By way of example, in some embodiments, the expression of PD-1, TIM3, LAG3, TIGIT, CD160, and 2B4/CD244/SLAMF4 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. By way of example, in some embodiments, the expression of PD-1, TIM3, LAG3, TIGIT, CD160, and CTLA-4 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. In certain embodiments, the expression of PD-1 and the expression of at least five additional immune checkpoint receptors are decreased at the RNA level relative to the expression of the corresponding marker by the exhausted T cells. In some embodiments, the expression of PD-1 and the expression of at least five additional immune checkpoint receptors are decreased at the protein level relative to the expression of the corresponding marker by the exhausted T cells. In certain embodiments, the expression of PD-1 and the expression of at least five additional immune checkpoint receptors are decreased at the RNA level and the protein level relative to the expression of the corresponding marker by the exhausted T cells.

In certain embodiments, the expression of PD-1 is decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells and expression of at least five additional immune checkpoint receptors are also decreased relative to the expression of the corresponding marker by the exhausted T cells. By way of example, in some embodiments, the expression of PD-1, LAG3, TIGIT, CD160, CTLA-4, and 2B4/CD244/SLAMF4 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. In some embodiments, the expression of PD-1, TIM3, TIGIT, CD160, CTLA-4, and 2B4/CD244/SLAMF4 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. In some embodiments, the expression of PD-1, TIM3, LAG3, CD160, CTLA-4, and 2B4/CD244/SLAMF4 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. By way of example, in some embodiments, the expression of PD-1, LAG3, TIGIT, CD160, CTLA-4, and 2B4/CD244/SLAMF4 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells By way of example, in some embodiments, the expression of PD-1, TIM3, LAG3, TIGIT, CTLA-4, and 2B4/CD244/SLAMF4 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. By way of example, in some embodiments, the expression of PD-1, TIM3, LAG3, TIGIT, CD160, and 2B4/CD244/SLAMF4 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. By way of example, in some embodiments, the expression of PD-1, TIM3, LAG3, TIGIT, CD160, and CTLA-4 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. In certain embodiments, the expression of PD-1 and the expression of at least five additional immune checkpoint receptors are decreased at the RNA level relative to the expression of the corresponding marker by the exhausted T cells. In some embodiments, the expression of PD-1 and the expression of at least five additional immune checkpoint receptors are decreased at the protein level relative to the expression of the corresponding marker by the exhausted T cells. In certain embodiments, the expression of PD-1 and the expression of at least five additional immune checkpoint receptors are decreased at the RNA level and the protein level relative to the expression of the corresponding marker by the exhausted T cells.

In certain embodiments, the expression of PD-1 is decreased by at least 1.1 fold, at least 1.25 fold, at least 1.5 fold, at least 2.0 fold, at least 2.5 fold, at least 3.0 fold, or at least 4.0 fold in the mitochondria replaced T cells and expression of at least six additional immune checkpoint receptors are also decreased. By way of example, in some embodiments, the expression of PD-1, TIM3, LAG3, TIGIT, CD160, CTLA-4, and 2B4/CD244/SLAMF4 are decreased by at least 1.1 fold, at least 1.25 fold, at least 1.5 fold, at least 2.0 fold, at least 2.5 fold, at least 3.0 fold, or at least 4.0 fold in the mitochondria replaced T cells, relative to the expression of the corresponding marker by the exhausted T cells. In certain embodiments, the expression of PD-1 and the expression of at least six additional immune checkpoint receptors are decreased at the RNA level relative to the expression of the corresponding marker by the exhausted T cells. In some embodiments, the expression of PD-1 and the expression of at least six additional immune checkpoint receptors are decreased at the protein level relative to the expression of the corresponding marker by the exhausted T cells. In certain embodiments, the expression of PD-1 and the expression of at least six additional immune checkpoint receptors are decreased at the RNA level and the protein level relative to the expression of the corresponding marker by the exhausted T cells.

In certain embodiments, the expression of PD-1 is decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells and expression of at least six additional immune checkpoint receptors are also decreased. By way of example, in some embodiments, the expression of PD-1, TIM3, LAG3, TIGIT, CD160, CTLA-4, and 2B4/CD244/SLAMF4 are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% in the mitochondria replaced T cells, relative to the expression of the corresponding marker by the exhausted T cells. In certain embodiments, the expression of PD-1 and the expression of at least six additional immune checkpoint receptors are decreased at the RNA level relative to the expression of the corresponding marker by the exhausted T cells. In some embodiments, the expression of PD-1 and the expression of at least six additional immune checkpoint receptors are decreased at the protein level relative to the expression of the corresponding marker by the exhausted T cells. In certain embodiments, the expression of PD-1 and the expression of at least six additional immune checkpoint receptors are decreased at the RNA level and the protein level relative to the expression of the corresponding marker by the exhausted T cells.

It is understood that mitochondria replaced T cell in which the expression of programmed cell death-1 (PD-1) and, in some embodiments, the expression of one, two, three or more other markers of T cell exhaustion, are decreased by at least 1.1 fold, at least 1.25 fold, at least 1.5 fold, at least 2.0 fold, at least 2.5 fold, at least 3.0 fold, or at least 4.0 fold relative to the expression of PD-1 by the exhausted T cells from which the mitochondria replaced T cells were produced encompasses both expression on the T cells, as well as expression within the T cells. It is understood that mitochondria replaced T cell in which the expression of programmed cell death-1 (PD-1) and, in some embodiments, the expression of one, two, three or more other markers of T cell exhaustion, are decreased by at least 5%, at least 10%, at least 15%, at least 20%, at least 25%, at least 30%, at least 35%, at least 40%, at least 45%, at least 50%, at least 60%, at least 70%, at least 80%, at least 90%, at least 95% relative to the expression of PD-1 by the exhausted T cells from which the mitochondria replaced T cells were produced encompasses both expression on the T cells, as well as expression within the T cells. In some embodiments, the expression of one, two, three or more other markers of T cell exhaustion, are decreased on the mitochondria replaced T cell, relative to the expression of PD-1 and, in some embodiments, the expression of the corresponding one, two, three or more other markers of T cell exhaustion on the exhausted T cells from which the mitochondria replaced T cells were produced. In certain embodiments, the expression of PD-1, and, in some embodiments, the expression of one, two, three or more other markers of T cell exhaustion, are decreased in the mitochondria replaced T cell, relative to the expression of PD-1 and, in some embodiments, the expression of the corresponding one, two, three or more other markers of T cell exhaustion in the exhausted T cells from which the mitochondria replaced T cells were produced relative to the expression of the corresponding marker by the exhausted T cells.

In some embodiments, the marker of T cell exhaustion is an intracellular protein. Non-limiting examples of intracellular proteins suitable as a markers of T cell exhaustion include the transcription factors TOX, TOX2, and NR4A. Accordingly, in some embodiments, the expression of TOX, TOX2, and/or NR4A is decreased by at least 2 fold in the mitochondria replaced T cells, relative to the expression of the corresponding marker in the exhausted T cells. In some embodiments, the expression of TOX, TOX2, and/or NR4A is decreased by at least 5 fold in the mitochondria replaced T cells, relative to the expression of the corresponding marker in the exhausted T cells. In some embodiments, the expression of TOX, TOX2, and/or NR4A is decreased by at least 10 fold in the mitochondria replaced T cells, relative to the expression of the corresponding marker in the exhausted T cells. In some embodiments, the expression of TOX, TOX2, and/or NR4A is decreased by at least 15 fold in the mitochondria replaced T cells, relative to the expression of the corresponding marker in the exhausted T cells. In some embodiments, the expression of TOX, TOX2, and/or NR4A is decreased by at least 20 fold in the mitochondria replaced T cells, relative to the expression of the corresponding marker in the exhausted T cells. In some embodiments, the expression of TOX, TOX2, and/or NR4A is decreased by more than 20 fold in the mitochondria replaced T cells, relative to the expression of the corresponding marker in the exhausted T cells.

In some embodiments, the expression of TOX, TOX2, and/or NR4A is decreased by at least 20% in the mitochondria replaced T cells, relative to the expression of the corresponding marker in the exhausted T cells. In some embodiments, the expression of TOX, TOX2, and/or NR4A is decreased by at least 30% in the mitochondria replaced T cells, relative to the expression of the corresponding marker in the exhausted T cells. In some embodiments, the expression of TOX, TOX2, and/or NR4A is decreased by at least 40% in the mitochondria replaced T cells, relative to the expression of the corresponding marker in the exhausted T cells. In some embodiments, the expression of TOX, TOX2, and/or NR4A is decreased by at least 50% in the mitochondria replaced T cells, relative to the expression of the corresponding marker in the exhausted T cells. In some embodiments, the expression of TOX, TOX2, and/or NR4A is decreased by at least 60% in the mitochondria replaced T cells, relative to the expression of the corresponding marker in the exhausted T cells. In some embodiments, the expression of TOX, TOX2, and/or NR4A is decreased by more than 70% in the mitochondria replaced T cells, relative to the expression of the corresponding marker in the exhausted T cells. In some embodiments, the expression of TOX, TOX2, and/or NR4A is decreased by more than 80% in the mitochondria replaced T cells, relative to the expression of the corresponding marker in the exhausted T cells. In some embodiments, the expression of TOX, TOX2, and/or NR4A is decreased by more than 90% in the mitochondria replaced T cells, relative to the expression of the corresponding marker in the exhausted T cells. In certain embodiments, the expression is decreased at the RNA level relative to the expression of the corresponding marker by the exhausted T cells. In some embodiments, the expression is decreased at the protein level relative to the expression of the corresponding marker by the exhausted T cells. In certain embodiments, the expression is decreased at the RNA level and the protein level relative to the expression of the corresponding marker by the exhausted T cells.

In some embodiments, the exhausted T cells exhibit one or more markers of senescence. Examples of markers of senescence include decreased expression of co-stimulatory molecules (e.g., CD27, and/or CD28); increased expression of KLRG-1 and/or CD57; upregulation of G1-regulating proteins (e.g., p15, p16, and p21); downregulation of Cdk2 and cyclin D3 expression; decreased Cdk2 and Cdk6 kinase activity; loss of human telomerase RNA component (hTERC) expression; decreased telomerase activity; increased expression of TIGIT; or a combination thereof. Senescent cells also generally exhibit one or more phenotypic marker, such as increased secretion of inflammatory cytokines (e.g., interferon gamma (IFNγ) and/or tumor necrosis factor alpha (TNFα), growth factors, and proteases, as well as reduced and/or slower rates of cell population doublings, shortened telomeres, increased DNA damage response (DDR), or a combination thereof.

As provided herein, the mitochondria replaced T cells generated according to the methods described herein, such as the methods described in Section 6.1 exhibit one, two or more improvements in effector function relative to the exhausted T cells from which the mitochondria replaced T cells are generated. See, e.g., Section 6.3 for effector functions of T cells that may be improved in mitochondria replaced T cells.

In certain embodiments, the improved effector function comprises increased proliferation, increased cytotoxicity, increased secretion of cytokines, or a combination thereof. In specific embodiments, the improved effector function comprises increased proliferation. In certain embodiments, the improved effector function comprises increased cytotoxicity. In some embodiments, the improved effector function comprises increased secretion of cytokines.

As provided herein, in one aspect, a method for producing mitochondria replaced T cells from exhausted T cells involves electroporating exhausted T cells with a nucleic acid sequence comprising a nucleotide sequence encoding XbaIR to reduce endogenous mitochondrial DNA (mtDNA) copy number. In some embodiments, the nucleotide sequence encoding XbaIR comprises DNA. In some embodiments, the nucleotide sequence encoding XbaIR comprises RNA. As provided herein, in one aspect, a method for producing mitochondria replaced T cells from exhausted T cells involves electroporating exhausted T cells with a nucleic acid sequence comprising a nucleotide sequence encoding a fusion protein comprising a mitochondrial-targeted sequence (MTS) and XbaIR to reduce endogenous mitochondrial DNA (mtDNA) copy number. In some embodiments, the nucleic acid sequence is RNA (e.g., mRNA). In some embodiments, the RNA is unmodified RNA. In some embodiments, the RNA is modified RNA. In some embodiments, the nucleic acid sequence is DNA (e.g., cDNA). In some embodiments, the DNA is unmodified DNA. In some embodiments, the DNA is modified DNA. Non-limiting examples of modified RNA or modified DNA include tritylated bases and unusual bases such as inosine.

Various methods for introducing the nucleotide (e.g., in a plasmid DNA expression vector cassette or as mRNA) are known in the art. In some embodiments, the nucleotide is introduced by electroporation. In specific embodiments, the electroporation method is flow electroporation, such as MaxCyte Flow Electroporation. In other specific embodiments, the electroporation method includes the nucleofection technology, such as Lonza's Nucleofector™ technology. However, it is understood that the methods described above for introducing the nucleotide are non-limiting and merely intended to be exemplary methods, and that any method known in the art can be used for introducing the nucleotide. For example, in some embodiments the nucleotide is introduced by cationic lipid transfection, or any other means that introduces the nucleotide into the cell. In some embodiments, the nucleotide is introduced by viral transduction.

In certain embodiments, the MaxCyte electroporator can be used for mRNA transfection, particularly in the clinical setting, which has cleared the standards of Good Manufacturing Practice and Good Clinical Practice. The transfection can be performed using the MaxCyte electroporator according to the manufacturer's protocol. It is further understood that the methods described above are merely exemplary and that any means of introducing mRNA can be used.

The specific targeting of the endonuclease to the mitochondria can be performed by incorporating a mitochondrial targeting sequence (MTS) adjacent to the endonuclease coding sequence, which will result in a fusion protein that targets the mitochondria. Strong MTSs have been identified and shown capable of targeting proteins to specific compartments when fused on their N-termini, and are termed mitochondrial targeting sequences. MTS suitable for the methods of the present invention are well known to the person skilled in the art (see, e.g., U.S. Pat. No. 8,039,587B2, which is hereby incorporated by reference in its entirety). For example, MTS to the mitochondrial matrix can be used, such as the MTS that is a targeting peptide from the cytochrome c oxidase subunit IV (COX 4), subunit VIII (COX 8), or subunit X (COX 10). In principle, any target sequence derived from any nuclear encoded mitochondrial matrix or inner membrane enzyme or an artificial sequence that is capable of rendering the fusion protein into a mitochondrial imported protein (hydrophobic moment greater than 5.5, at least two basic residues, amphiphilic alpha-helical conformation; see, e.g., Bedwell et al., Mol Cell Biol. 9(3) (1989), 1014-1025) is useful for the purposes of the present invention.

In certain embodiments, the MTS is a human MTS. In another embodiment, the MTS is from another species. Non-limiting examples of such sequences are the cytochrome c oxidase subunit X (COX 10) MTS (MAASPHTLSSRLLTGCVGGSVWYLERRT) (SEQ ID NO:1), and the cytochrome c oxidase subunit VIII (COX 8) MTS (MSVLTPLLLRSLTGSARRLMVPRA) (SEQ ID NO:2). Additional non-limiting examples of MTS sequences are the natural MTS of each individual mitochondrial protein that is encoded by the nuclear DNA, translated (produced) in the cytoplasm and transported into the mitochondria, as well as citrate synthase (cs), lipoamide deydrogenase (LAD), and C6ORF66 (ORF). The various MTS may be exchangeable for each mitochondrial enzyme among themselves. Accordingly, in some embodiments, the MTS targets a mitochondrial matrix protein. In specific embodiments, the mitochondrial matrix protein is subunit VIII of human cytochrome C oxidase.

In certain aspects, the methods provided herein involve culturing the exhausted T cells transfected or transformed, or otherwise comprising the nucleic acid sequence comprising a nucleotide sequence encoding XbaIR (in specific embodiments, the nucleic acid sequence comprises a nucleotide sequence encoding a fusion protein comprising a mitochondrial-targeted sequence (MTS) and XbaIR), to reduce endogenous mitochondrial DNA (mtDNA) copy number. In specific aspects, the methods provided herein involve culturing the exhausted T cells transfected or transformed, or otherwise comprising the nucleic acid sequence comprising a nucleotide sequence encoding XbaIR to reduce endogenous mitochondrial DNA (mtDNA) copy number with minimal cytotoxicity and until the cells are at least substantially free of the nucleotide sequence encoding XbaIR. In specific aspects, the methods provided herein involve culturing the exhausted T cells transfected or transformed, or otherwise comprising the nucleic acid sequence comprising a nucleotide sequence encoding XbaIR to reduce endogenous mitochondrial DNA (mtDNA) copy number with minimal cytotoxicity and until the cells are at least substantially free of the XbaIR. Techniques known to one of skill in the art or described herein may be used to measure cytotoxicity. Techniques known to one of skill in the art or described herein (e.g., in the Example) may be used to assess the amount of the nucleotide encoding XbaIR (e.g., PCR or qPCR), or the amount of the XbaIR or the fusion protein (e.g., immunoblot).

In some embodiments, the sufficient period of time to reduce endogenous mitochondrial DNA (mtDNA) copy number is about one day to about ten days. In some embodiments, the sufficient period of time to reduce endogenous mitochondrial DNA (mtDNA) copy number is about two days to about seven days. In some embodiments, the sufficient period of time to reduce endogenous mitochondrial DNA (mtDNA) copy number is about one day to about five days. In some embodiments, the sufficient period of time to reduce endogenous mitochondrial DNA (mtDNA) copy number is about two days to about four days. In some embodiments, the sufficient period of time to reduce endogenous mitochondrial DNA (mtDNA) copy number is about two days to about three days. In some embodiments, the sufficient period of time to reduce endogenous mitochondrial DNA (mtDNA) copy number is about one day to about four days. In some embodiments, the sufficient period of time to reduce endogenous mitochondrial DNA (mtDNA) copy number is about one day to about three days. In some embodiments, the sufficient period of time to reduce endogenous mitochondrial DNA (mtDNA) copy number is about two days to about seven days. In some embodiments, the sufficient period of time to reduce endogenous mitochondrial DNA (mtDNA) copy number is about one day. In some embodiments, the sufficient period of time to reduce endogenous mitochondrial DNA (mtDNA) copy number is about two days. In some embodiments, the sufficient period of time to reduce endogenous mitochondrial DNA (mtDNA) copy number is about three days. In some embodiments, the sufficient period of time to reduce endogenous mitochondrial DNA (mtDNA) copy number is about four days. In some embodiments, the sufficient period of time to reduce endogenous mitochondrial DNA (mtDNA) copy number is about five days. In some embodiments, the sufficient period of time to reduce endogenous mitochondrial DNA (mtDNA) copy number is about six days. In some embodiments, the sufficient period of time to reduce endogenous mitochondrial DNA (mtDNA) copy number is about seven days. In some embodiments, the sufficient period of time to reduce endogenous mitochondrial DNA (mtDNA) copy number is about eight days. In some embodiments, the sufficient period of time to reduce endogenous mitochondrial DNA (mtDNA) copy number is about nine days. In some embodiments, the sufficient period of time to reduce endogenous mitochondrial DNA (mtDNA) copy number is about ten days.

In certain aspects, the methods provided herein involve incubation of the exhausted T cells with the isolated exogenous mitochondria in the presence of an effective amount of rapamycin or a derivative thereof. In some embodiments, the rapamycin or a derivative thereof is a concentration of about 100 nanomolar (nM) to about 1000 nM. In some embodiments, the rapamycin or a derivative thereof is a concentration of about 200 nM to about 500 nM. In some embodiments, the rapamycin or a derivative thereof is a concentration of about 300 nM to about 600 nM. In some embodiments, the rapamycin or a derivative thereof is a concentration of about 400 nM to about 700 nM.

In certain embodiments, the effective amount of rapamycin or a derivative thereof is a concentration of about 100 nM. In some embodiments, the effective amount of rapamycin or a derivative thereof is a concentration of about 150 nM. In some embodiments, the effective amount of rapamycin or a derivative thereof is a concentration of about 200 nM. In some embodiments, the effective amount of rapamycin or a derivative thereof is a concentration of about 250 nM. In some embodiments, the effective amount of rapamycin or a derivative thereof is a concentration of about 300 nM. In some embodiments, the effective amount of rapamycin or a derivative thereof is a concentration of about 350 nM. In some embodiments, the effective amount of rapamycin or a derivative thereof is a concentration of about 400 nM. In some embodiments, the effective amount of rapamycin or a derivative thereof is a concentration of about 450 nM. In some embodiments, the effective amount of rapamycin or a derivative thereof is a concentration of about 500 nM. In some embodiments, the effective amount of rapamycin or a derivative thereof is a concentration of about 550 nM. In some embodiments, the effective amount of rapamycin or a derivative thereof is a concentration of about 600 nM. In some embodiments, the effective amount of rapamycin or a derivative thereof is a concentration of about 650 nM. In some embodiments, the effective amount of rapamycin or a derivative thereof is a concentration of about 700 nM. In some embodiments, the effective amount of rapamycin or a derivative thereof is a concentration of about 750 nM. In some embodiments, the effective amount of rapamycin or a derivative thereof is a concentration of about 800 nM. In some embodiments, the effective amount of rapamycin or a derivative thereof is a concentration of about 850 nM. In some embodiments, the effective amount of rapamycin or a derivative thereof is a concentration of about 900 nM. In some embodiments, the effective amount of rapamycin or a derivative thereof is a concentration of about 1000 nM. In some embodiments, the effective amount of rapamycin or a derivative thereof is a concentration greater than 1000 nM.

Non-limiting examples of rapamycin derivatives (e.g., rapamycin analogs, also known as “rapalogs”) include, for example, temsirolimus (CAS Number 162635-04-3; C56H87NO16), everolimus (CAS Number 159351-69-6; C53H83NO14), ridaforolimus (CAS Number 572924-54-0; C53H84NO14P), WYE-125132 (WYE-132), and Zotarolimus (ABT-578).

In specific embodiments, the methods provided herein involve incubation of the exhausted T cells with the isolated exogenous mitochondria in the presence of an effective amount of rapamycin. In some embodiments, the rapamycin is a concentration of about 100 nanomolar (nM) to about 1000 nM. In some embodiments, the rapamycin is a concentration of about 200 nM to about 500 nM. In some embodiments, the rapamycin is a concentration of about 300 nM to about 600 nM. In some embodiments, the rapamycin is a concentration of about 400 nM to about 700 nM. In certain embodiments, the effective amount of rapamycin is a concentration of about 100 nM. In some embodiments, the effective amount of rapamycin is a concentration of about 150 nM. In some embodiments, the effective amount of rapamycin is a concentration of about 200 nM. In some embodiments, the effective amount of rapamycin is a concentration of about 250 nM. In some embodiments, the effective amount of rapamycin is a concentration of about 300 nM. In some embodiments, the effective amount of rapamycin is a concentration of about 350 nM. In some embodiments, the effective amount of rapamycin is a concentration of about 400 nM. In some embodiments, the effective amount of rapamycin is a concentration of about 450 nM. In some embodiments, the effective amount of rapamycin is a concentration of about 500 nM. In some embodiments, the effective amount of rapamycin is a concentration of about 550 nM. In some embodiments, the effective amount of rapamycin is a concentration of about 600 nM. In some embodiments, the effective amount of rapamycin is a concentration of about 650 nM. In some embodiments, the effective amount of rapamycin is a concentration of about 700 nM. In some embodiments, the effective amount of rapamycin is a concentration of about 750 nM. In some embodiments, the effective amount of rapamycin is a concentration of about 800 nM. In some embodiments, the effective amount of rapamycin is a concentration of about 850 nM. In some embodiments, the effective amount of rapamycin is a concentration of about 900 nM. In some embodiments, the effective amount of rapamycin is a concentration of about 1000 nM. In some embodiments, the effective amount of rapamycin is a concentration greater than 1000 nM.

The amount of the isolated exogenous mitochondria that is incubated with the exhausted T cell to generate mitochondria replaced T cells will depend on factors, such as the amount of exhausted T cells that are being co-incubated with the isolated mitochondria. Generally, the amount of isolated exogenous mitochondria incubated with the exhausted T cells is about 5 μg to about 100 μg per 1×10⁶ T cells. In some embodiments, the amount of isolated exogenous mitochondria incubated with the exhausted T cells is about 10 μg to about 90 μg per 1×10⁶ T cells. In some embodiments, the amount of isolated exogenous mitochondria incubated with the exhausted T cells is about 20 μg to about 80 μg per 1×10⁶ T cells. In some embodiments, the amount of isolated exogenous mitochondria incubated with the exhausted T cells is about 30 μg to about 70 μg per 1×10⁶ T cells. In some embodiments, the amount of isolated exogenous mitochondria incubated with the exhausted T cells is about 5 μg per 1×10⁶ T cells. In some embodiments, the amount of isolated exogenous mitochondria incubated with the exhausted T cells is about 10 μg per 1×10⁶ T cells. In some embodiments, the amount of isolated exogenous mitochondria incubated with the exhausted T cells is about 20 μg per 1×10⁶ T cells. In some embodiments, the amount of isolated exogenous mitochondria incubated with the exhausted T cells is about 30 μg per 1×10⁶ T cells. In some embodiments, the amount of isolated exogenous mitochondria incubated with the exhausted T cells is about 40 μg per 1×10⁶ T cells. In some embodiments, the amount of isolated exogenous mitochondria incubated with the exhausted T cells is about 50 μg per 1×10⁶ T cells. In some embodiments, the amount of isolated exogenous mitochondria incubated with the exhausted T cells is about 60 μg per 1×10⁶ T cells. In some embodiments, the amount of isolated exogenous mitochondria incubated with the exhausted T cells is about 70 μg per 1×10⁶ T cells. In some embodiments, the amount of isolated exogenous mitochondria incubated with the exhausted T cells is about 80 μg per 1×10⁶ T cells. In some embodiments, the amount of isolated exogenous mitochondria incubated with the exhausted T cells is about 90 μg per 1×10⁶ T cells. In some embodiments, the amount of isolated exogenous mitochondria incubated with the exhausted T cells is about 100 μg per 1×10⁶ T cells. In some embodiments, the amount of isolated exogenous mitochondria incubated with the exhausted T cells is greater than 100 μg per 1×10⁶ T cells.

As provided herein, the isolated exogenous mitochondria of the present disclosure can be obtained from various types of cells that have a healthy, and functional mitochondria. Assays for determining mitochondrial function are known in the art, and include assays such as those described in Section 6.3. Exemplary sources of mitochondria for use in the methods provided herein include fibroblasts, platelet cells, as well as other lymphoid cells. In certain embodiments, the isolated exogenous mitochondria are obtained from a fibroblast. In some embodiments, the isolated exogenous mitochondria are obtained from a platelet cell. In some embodiments, the isolated exogenous mitochondria are obtained from a lymphoid cell.

As provided herein, the isolated exogenous mitochondria can be autologous or allogeneic to the recipient cell. In some embodiments, the isolated exogenous mitochondria is allogeneic, relative to the recipient cell. For example, the isolated exogenous mitochondria can be obtained from a subject that is different from the recipient cell. In other embodiments, the isolated exogenous mitochondria is autologous. By way of example, an exemplary autologous isolated exogenous mitochondria can include mitochondria isolated from the same subject at an earlier point in time, such as from the placenta or umbilical cord blood. Another exemplary autologous exogenous mtDNA can include, for example, donor mtDNA that has been isolated from the same subject as the recipient cell and modified prior to replacing it with the recipient cell.

Mitochondrial isolation may be accomplished by any of a number of well-known techniques including, but not limited to those described herein. In certain embodiments, the exogenous mitochondria for use in mitochondrial transfer is isolated using a commercial kit, such as, for example, the Qproteum mitochondria isolation kit (Qiagen, USA), or the MITOISO2 mitochondria isolation kit (Sigma, USA). In other embodiments, the exogenous mitochondria for use in mitochondrial transfer is isolated manually (see, e.g., Preble et al. J. Vis. Exp. 2014, 91: e51682; Gasnier et al. Anal Biochem 1993; 212(1):173-8 and Frezza et al. Nat Protoc 2007; 2(2): 287-95). For example, an exemplary manual isolation of mitochondria includes isolating the mitochondria from donor cells by pelleting the donor cells, washing the cell pellet of 1-2 mL derived from approximately 10⁹ cells grown in culture, swelling the cells in a hypotonic buffer, rupturing the cells with a Dounce or Potter-Elvehjem homogenizer using a tight-fitting pestle, and isolating the mitochondria by differential centrifugation. Manual isolation can also include, for example, sucrose density gradient ultracentrifugation, or free-flow electrophoresis. Without wishing to be bound by any particular method, it is understood that the kits and manual methods described herein are exemplary, and that any mitochondrial isolation method can be used, and would be within the skill set of a person skilled in the art. In a specific embodiment, mitochondria are isolated and transferred using the methodology described in Section 8.

In some embodiments, the isolated donor mitochondria is substantially pure of other organelles. In other embodiments, the isolated mitochondria can contain impurities and is enriched for mitochondria. For example, in some embodiments, the isolated mitochondria are about 90% pure, about 80% pure, about 70% pure, about 60%, pure, about 50%, pure, or any integer in-between. In general, it is understood that any impurities contained with the isolated donor mitochondria will not affect the viability or function of the recipient cell upon mitochondrial transfer. In specific embodiments, the transfer of the exogenous mitochondria, exogenous mtDNA, or a combination thereof does not involve transfer of non-mitochondrial organelles.

The quantity and quality of isolated mitochondria can easily be determined by a number of well-known techniques including but not limited to those described herein, and in the cited references. For example, in some embodiments, the quantity of isolated mitochondria is determined by assessment of total protein content. Various methods are available for assessing total protein content, such as the Biuret and Lowry procedures (see, e.g., Hartwig et al., Proteomics, 2009 June; 9(11):3209-14), as well as the Bradford protein assay (Bradford. Anal Biochem. 1976; 72:248-54)). In other embodiments, the quantity of isolated mitochondria is determined by mtDNA copy number.

The period of time sufficient to incubate the exhausted T cells having reduced endogenous mtDNA with the isolated exogenous mitochondria to generate mitochondria replaced T cells can be any period of time that results in a detectable amount of exogenous mtDNA that is greater than the exhausted T cells not subjected to exogenous mitochondria. In some embodiments, the period of time sufficient to incubate the exhausted T cells having reduced endogenous mtDNA with the isolated exogenous mitochondria to generate mitochondria replaced T cells is any period of time that results in at least 20% of exogenous mtDNA being transferred to the exhausted T cells relative to the amount of exogenous mtDNA transferred to exhausted T cells not incubated with the exogenous mtDNA. In some embodiments, the period of time sufficient to incubate the exhausted T cells having reduced endogenous mtDNA with the isolated exogenous mitochondria to generate mitochondria replaced T cells is any period of time that results in at least 30% of exogenous mtDNA being transferred to the exhausted T cells relative to the amount of exogenous mtDNA transferred to exhausted T cells not incubated with the exogenous mtDNA. In some embodiments, the period of time sufficient to incubate the exhausted T cells having reduced endogenous mtDNA with the isolated exogenous mitochondria to generate mitochondria replaced T cells is any period of time that results in at least 40% of exogenous mtDNA being transferred to the exhausted T cells relative to the amount of exogenous mtDNA transferred to exhausted T cells not incubated with the exogenous mtDNA. In some embodiments, the period of time sufficient to incubate the exhausted T cells having reduced endogenous mtDNA with the isolated exogenous mitochondria to generate mitochondria replaced T cells is any period of time that results in at least 50% of exogenous mtDNA being transferred to the exhausted T cells relative to the amount of exogenous mtDNA transferred to exhausted T cells not incubated with the exogenous mtDNA. In some embodiments, the period of time sufficient to incubate the exhausted T cells having reduced endogenous mtDNA with the isolated exogenous mitochondria to generate mitochondria replaced T cells is any period of time that results in at least 60% of exogenous mtDNA being transferred to the exhausted T cells relative to the amount of exogenous mtDNA transferred to exhausted T cells not incubated with the exogenous mtDNA. In some embodiments, the period of time sufficient to incubate the exhausted T cells having reduced endogenous mtDNA with the isolated exogenous mitochondria to generate mitochondria replaced T cells is any period of time that results in at least 70% of exogenous mtDNA being transferred to the exhausted T cells relative to the amount of exogenous mtDNA transferred to exhausted T cells not incubated with the exogenous mtDNA. In some embodiments, the period of time sufficient to incubate the exhausted T cells having reduced endogenous mtDNA with the isolated exogenous mitochondria to generate mitochondria replaced T cells is any period of time that results in a majority (i.e., greater than 50%), about 20% to about 95%, about 25% to about 90%, about 30% to about 85%, about 35% to about 80%, about 40% to about 75%, about 30% to about 60%, 40% to about 70%, about 40% to about 85%. about 40% to about 80%, about 50% to about 80%, about 60% to about 90%, about 65% to about 95% of exogenous mtDNA being transferred to the exhausted T cells relative to the amount of exogenous mtDNA transferred to exhausted T cells not incubated with the exogenous mtDNA. Techniques known to one of skill in the art or described herein (e.g., in the Example) may be used to assess the transfer of exogenous mtDNA to the exhausted T cells.

Generally, the sufficient period of time is at least approximately 12 hours and less than two weeks. In some embodiments, the sufficient period of time is at least 12 hours. In some embodiments, the sufficient period of time is at least 24 hours. In some embodiments, the sufficient period of time is at least 36 hours. In some embodiments, the sufficient period of time is at least 48 hours. In some embodiments, the sufficient period of time is approximately 2 days or more. In some embodiments, the sufficient period of time is approximately 7 days or more. In some embodiments, the sufficient period of time is approximately 2 days to approximately 7 days. In some embodiments, the sufficient period of time is approximately 1 day to approximately 7 days.

By way of example, mitochondria transfer can also be performed using simple co-incubation of the exhausted T cells having reduced endogenous mitochondria DNA (mtDNA) and the isolated exogenous mitochondria. However, it is also possible to promote the transfer of mitochondria by optionally centrifuging the T cells and the isolated exogenous mitochondria. Other means of mitochondria transfer include the use of thermal shock, injection, and/or nanoblades.

Centrifugation conditions can readily by determined by a person skilled in the art, and the speed and time can vary so long as the cell and mitochondria are not damaged, and transfer of the mitochondria is promoted. By way of example, centrifuging conditions can include centrifuging at approximately 1,500 relative centrifugal force (RCF, also expressed as “g”) for approximately 5 minutes at room temperature. In a specific embodiment, centrifuging is as described in the Example, infra.

In one embodiment, centrifuging is at approximately 500 RCF for approximately 5 minutes at room temperature. In another embodiment, centrifuging is at approximately 750 RCF for approximately 5 minutes at room temperature. In another embodiment, centrifuging is at approximately 1,000 RCF for approximately 5 minutes at room temperature. In another embodiment, centrifuging is at approximately 1,500 RCF for approximately 5 minutes at room temperature. In another embodiment, centrifuging is at approximately 2,000 RCF for approximately 5 minutes at room temperature. In another embodiment, centrifuging is at approximately 2,500 RCF for approximately 5 minutes at room temperature. In another embodiment, centrifuging is at approximately 3,000 RCF for approximately 5 minutes at room temperature.

In one embodiment, centrifuging is at approximately 500 RCF for approximately 10 minutes at room temperature. In another embodiment, centrifuging is at approximately 750 RCF for approximately 10 minutes at room temperature. In another embodiment, centrifuging is at approximately 1,000 RCF for approximately 10 minutes at room temperature. In another embodiment, centrifuging is at approximately 1,500 RCF for approximately 10 minutes at room temperature. In another embodiment, centrifuging is at approximately 2,000 RCF for approximately 10 minutes at room temperature. In another embodiment, centrifuging is at approximately 2,500 RCF for approximately 10 minutes at room temperature. In another embodiment, centrifuging is at approximately 3,000 RCF for approximately 10 minutes at room temperature.

In another embodiment, centrifuging is at approximately 500 RCF for approximately 15 minutes at room temperature. In another embodiment, centrifuging is at approximately 750 RCF for approximately 15 minutes at room temperature. In another embodiment, centrifuging is at approximately 1,000 RCF for approximately 15 minutes at room temperature. In another embodiment, centrifuging is at approximately 1,500 RCF for approximately 15 minutes at room temperature. In another embodiment, centrifuging is at approximately 2,000 RCF for approximately 15 minutes at room temperature. In another embodiment, centrifuging is at approximately 2,500 RCF for approximately 15 minutes at room temperature. In another embodiment, centrifuging is at approximately 3,000 RCF for approximately 15 minutes at room temperature.

In another embodiment, centrifuging is at approximately 500 RCF for less than an hour at room temperature. In another embodiment, centrifuging is at approximately 750 RCF for less than an hour at room temperature. In another embodiment, centrifuging is at approximately 1,000 RCF for less than an hour at room temperature. In another embodiment, centrifuging is at approximately 1,500 RCF for less than an hour at room temperature. In another embodiment, centrifuging is at approximately 2,000 RCF for less than an hour at room temperature. In another embodiment, centrifuging is at approximately 2,500 RCF for less than an hour at room temperature. In another embodiment, centrifuging is at approximately 3,000 RCF for less than an hour at room temperature.

In one embodiment, centrifuging is at approximately 500 RCF for approximately 5 minutes at about 4° ° C. In another embodiment, centrifuging is at approximately 750 RCF for approximately 5 minutes at about 4° C. In another embodiment, centrifuging is at approximately 1,000 RCF for approximately 5 minutes at about 4° C. In another embodiment, centrifuging is at approximately 1,500 RCF for approximately 5 minutes at about 4° C. In another embodiment, centrifuging is at approximately 2,000 RCF for approximately 5 minutes at about 4° C. In another embodiment, centrifuging is at approximately 2,500 RCF for approximately 5 minutes at about 4ºC. In another embodiment, centrifuging is at approximately 3,000 RCF for approximately 5 minutes at about 4° C.

In another embodiment, centrifuging is at approximately 500 RCF for approximately 10 minutes at about 4° C. In another embodiment, centrifuging is at approximately 750 RCF for approximately 10 minutes at about 4° C. In another embodiment, centrifuging is at approximately 1,000 RCF for approximately 10 minutes at about 4° C. In another embodiment, centrifuging is at approximately 1,500 RCF for approximately 10 minutes at about 4° C. In another embodiment, centrifuging is at approximately 2,000 RCF for approximately 10 minutes at about 4° ° C. In another embodiment, centrifuging is at approximately 2,500 RCF for approximately 10 minutes at about 4° C. In another embodiment, centrifuging is at approximately 3,000 RCF for approximately 10 minutes at about 4° C.

In another embodiment, centrifuging is at approximately 500 RCF for approximately 15 minutes at about 4° C. In another embodiment, centrifuging is at approximately 750 RCF for approximately 15 minutes at about 4° C. In another embodiment, centrifuging is at approximately 1,000 RCF for approximately 15 minutes at about 4° ° C. In another embodiment, centrifuging is at approximately 1,500 RCF for approximately 15 minutes at about 4° C. In another embodiment, centrifuging is at approximately 2,000 RCF for approximately 15 minutes at about 4° C. In another embodiment, centrifuging is at approximately 2,500 RCF for approximately 15 minutes at about 4° C. In another embodiment, centrifuging is at approximately 3,000 RCF for approximately 15 minutes at about 4° C.

In another embodiment, centrifuging is at approximately 500 RCF for less than an hour at about 4° C. In another embodiment, centrifuging is at approximately 750 RCF for less than an hour at about 4° C. In another embodiment, centrifuging is at approximately 1,000 RCF for less than an hour at about 4° C. In another embodiment, centrifuging is at approximately 1,500 RCF for less than an hour at about 4° C. In another embodiment, centrifuging is at approximately 2,000 RCF for less than an hour at about 4° C. In another embodiment, centrifuging is at approximately 2,500 RCF for less than an hour at about 4° C. In another embodiment, centrifuging is at approximately 3,000 RCF for less than an hour at about 4° C.

In some embodiments, the ratio of the copy number of exogenous mtDNA to the copy number of endogenous mtDNA in the mitochondria replaced T cell generated according to the methods provided herein is greater than 4 to 1. In some embodiments, the ratio is about 4 to 1. In some embodiments, the ratio is about 3 to 1. In some embodiments, the ratio is about 2 to 1. In some embodiments, the ratio is about 1 to 1. In some embodiments, the ratio is about 0.75 to 1. In some embodiments, the ratio is about 0.5 to 1. In some embodiments, the ratio is about 0.25 to 1. In some embodiments, the ratio is about 0.1 to 1.

The level of endogenous mtDNA that is replaced accordingly to the methods provided herein need not result in a complete replacement of endogenous mtDNA with exogenous mtDNA (i.e., 100% replacement). For example, in some embodiments, at least 10% of the endogenous mtDNA has been replaced with exogenous mtDNA. In some embodiments, at least 15% of the endogenous mtDNA has been replaced with exogenous mtDNA. In some embodiments, at least 20% of the endogenous mtDNA has been replaced with exogenous mtDNA. In some embodiments, at least 25% of the endogenous mtDNA has been replaced with exogenous mtDNA. In some embodiments, at least 30% of the endogenous mtDNA has been replaced with exogenous mtDNA. In some embodiments, at least 35% of the endogenous mtDNA has been replaced with exogenous mtDNA. In some embodiments, at least 40% of the endogenous mtDNA has been replaced with exogenous mtDNA. In some embodiments, at least 45% of the endogenous mtDNA has been replaced with exogenous mtDNA. In some embodiments, at least 50% of the endogenous mtDNA has been replaced with exogenous mtDNA. In some embodiments, at least 55% of the endogenous mtDNA has been replaced with exogenous mtDNA. In some embodiments, at least 60% of the endogenous mtDNA has been replaced with exogenous mtDNA. In some embodiments, at least 65% of the endogenous mtDNA has been replaced with exogenous mtDNA. In some embodiments, at least 70% of the endogenous mtDNA has been replaced with exogenous mtDNA. In some embodiments, at least 80% of the endogenous mtDNA has been replaced with exogenous mtDNA. In some embodiments, at least 90% of the endogenous mtDNA has been replaced with exogenous mtDNA. In some embodiments, 100% of the endogenous mtDNA has been replaced with exogenous mtDNA. In some embodiments, a majority (i.e., greater than 50%) of the endogenous mtDNA has been replaced with exogenous mtDNA. In some embodiments, about 20% to about 95% of the endogenous mtDNA has been replaced with exogenous mtDNA. In some embodiments, about 25% to about 90% of the endogenous mtDNA has been replaced with exogenous mtDNA. In some embodiments, about 30% to about 85% of the endogenous mtDNA has been replaced with exogenous mtDNA. In some embodiments, about 35% to about 80% of the endogenous mtDNA has been replaced with exogenous mtDNA. In some embodiments, about 40% to about 75% of the endogenous mtDNA has been replaced with exogenous mtDNA. In some embodiments, about 20% to about 95% of the endogenous mtDNA has been replaced with exogenous mtDNA. In some embodiments, about 30% to about 60% of the endogenous mtDNA has been replaced with exogenous mtDNA. In some embodiments, about 40% to about 70% of the endogenous mtDNA has been replaced with exogenous mtDNA. In some embodiments, about 40% to about 85% of the endogenous mtDNA has been replaced with exogenous mtDNA. In some embodiments, about 40% to about 80% of the endogenous mtDNA has been replaced with exogenous mtDNA. In some embodiments, about 30% to about 85% of the endogenous mtDNA has been replaced with exogenous mtDNA. In some embodiments, about 50% to about 80% of the endogenous mtDNA has been replaced with exogenous mtDNA. In some embodiments, about 60% to about 90% of the endogenous mtDNA has been replaced with exogenous mtDNA. In some embodiments, about 65% to about 95% of the endogenous mtDNA has been replaced with exogenous mtDNA. Techniques known to one of skill in the art or described herein (e.g., in the Example) may be used to assess the replacement of endogenous mtDNA with exogenous mtDNA.

As provided herein, the level of endogenous mtDNA copy number that is reduced according to the methods provided herein need not be a complete depletion. In some embodiments, a majority (i.e., greater than 50%) of the endogenous mtDNA copy number is reduced. In some embodiments, about 20% to about 95% of the endogenous mtDNA copy number is reduced. In some embodiments, about 25% to about 90% of the endogenous mtDNA copy number is reduced. In some embodiments, about 30% to about 90% of the endogenous mtDNA copy number is reduced. In some embodiments, about 30% to about 85% of the endogenous mtDNA copy number is reduced. In some embodiments, about 35% to about 80% of the endogenous mtDNA copy number is reduced. In some embodiments, about 40% to about 75% of the endogenous mtDNA copy number is reduced. In some embodiments, about 20% to about 95% of the endogenous mtDNA copy number is reduced. In some embodiments, about 30% to about 60% of the endogenous mtDNA copy number is reduced. In some embodiments, about 40% to about 70% of the endogenous mtDNA copy number is reduced. In some embodiments, about 40% to about 85% of the endogenous mtDNA copy number is reduced. In some embodiments, about 40% to about 80% of the endogenous mtDNA copy number is reduced. In some embodiments, about 30% to about 85% of the endogenous mtDNA copy number is reduced. In some embodiments, about 50% to about 80% of the endogenous mtDNA copy number is reduced. In some embodiments, about 60% to about 80% of the endogenous mtDNA copy number is reduced. In some embodiments, about 60% to about 90% of the endogenous mtDNA copy number is reduced. In some embodiments, about 65% to about 95% of the endogenous mtDNA copy number is reduced. In some embodiments, about 10% of the endogenous mtDNA copy number is reduced. In some embodiments, about 10% of the endogenous mtDNA copy number is reduced. In some embodiments, about 20% of the endogenous mtDNA copy number is reduced. In some embodiments, about 30% of the endogenous mtDNA copy number is reduced. In some embodiments, about 40% of the endogenous mtDNA copy number is reduced. In some embodiments, about 50% of the endogenous mtDNA copy number is reduced. In some embodiments, about 60% of the endogenous mtDNA copy number is reduced. In some embodiments, about 70% of the endogenous mtDNA copy number is reduced. In some embodiments, about 80% of the endogenous mtDNA copy number is reduced. In some embodiments, about 85% of the endogenous mtDNA copy number is reduced. In some embodiments, about 90% of the endogenous mtDNA copy number is reduced. In some embodiments, about 95% of the endogenous mtDNA copy number is reduced. Techniques known to one of skill in the art or described herein (e.g., in the Example) may be used to assess the amount of endogenous mtDNA copy number before and after reduction.

In certain embodiments, the T cells are CD4+ T cells. In some embodiments, the T cells are CD8+ T cells. In certain embodiments, the T cells are double positive CD4+CD8+ T cells. In some embodiments, the T cells are or comprise Tregs. In certain embodiments, the T cells are or comprise effector T cells. In some embodiments, the T cells are or comprise memory T cells, effector T cells, Tregs, or a combination thereof.

In some embodiments, the T cells are T cells genetically modified to express a chimeric antigen receptor (CAR) or a T cell receptor (TCR). For example, TCRs use naturally occurring receptors that can also recognize antigens that are inside tumor cells. CARs, on the other hand, comprise portions of an antibody that can recognize a specific antigen only on the surface of cancer cells. Despite their use in immunotherapy CAR-T cells and TCR T cells can become exhausted. Thus, as provided herein, in some embodiments, the mitochondria replaced T cells produced from exhausted T cells according to the method described herein, such as the methods described in Section 6.1, can be CAR-T cells or TCR T cells, and be administered to a subject to treat or ameliorate a symptom of a cancer. Non-limiting exemplary types of cancer that can benefit from the mitochondria replaced T cells described herein include blood cancers (e.g., acute lymphatic leukemia, multiple myeloma, B cell lymphoma, mantel cell lymphoma), as well as solid tumors. In specific embodiments, the subject is a human subject.

CARs are generally designed to include an extracellular target-binding domain, a hinge region, a transmembrane domain that anchors the CAR to the cell membrane, and one or more intracellular domains that transmit activation signals. Depending on the number of costimulatory domains, CARs can be classified into first generation (an intracellular domain, e.g., CD3ζ, only), second generation (one costimulatory domain and an intracellular domain), or third generation CARs (more than one costimulatory domain and an intracellular domain). New generation CARs are also being developed (see, e.g., Guedan S, et al. Mol Ther Methods Clin Dev. 2018 Dec. 31; 12: 145-156). CAR targets for hematological malignancies (e.g., CD19, BCMA) and CAR targets for solid tumors (e.g., HER2, PSCA) are known in the art, and any target is suitable for use with the present disclosure (see, e.g., Dotti G, et al. Immunol Rev. 2014; 257(1): 107-126). CAR-T cells can be autologous or allogeneic to the subject receiving administration of the CAR-T cells. In some embodiments, the CAR-T cells are allogeneic, relative to the subject. In other embodiments, the CAR-T cells are autologous.

In certain embodiments, the CAR comprises a tumor antigen recognition domain, a transmembrane domain, and one or more intracellular signaling domains. In some embodiments, the CAR comprises a tumor antigen recognition domain, a transmembrane domain, one or more costimulatory molecules, and one or more intracellular signaling domains. In a specific embodiment, the CAR comprises a tumor antigen recognition domain, a transmembrane domain, and an intracellular domain. In a specific embodiment, the CAR comprises a tumor antigen recognition domain, a transmembrane domain, an intracellular domain and at least one costimulatory domain. In another specific embodiment, the CAR comprises a tumor antigen recognition domain, a transmembrane domain, two or more costimulatory domains and an intracellular domain. In some embodiments, the CAR comprises a constitutively or inducibly expressed chemokine. In certain embodiments, the CAR comprises intracellular domains of a cytokine receptor (e.g. IL-2Rβ chain fragment).

In some embodiments, the T cells are T cells genetically modified to express a TCR. TCRs generally use heterodimers consisting of alpha and beta peptide chains to recognize polypeptide fragments presented by MHC molecules, and the TCR T cells are genetically engineered TCR products that can recognize specific antigens. Generally, the artificially designed high-affinity TCR is encoded in T cells by genetic engineering technology, which enhances both specificity recognition and affinity during the recognition of tumor cells by T cells. TCR-T cells can be autologous or allogeneic to the subject receiving administration of the TCR-T cells. In some embodiments, the TCR-T cells are allogeneic, relative to the subject. In other embodiments, the TCR-T cells are autologous. Techniques known to one of skill in the art may be used to produce CAR-T cells and TCR T cells, such as for example, the Example for one method of producing CAR T cell]

In certain embodiments, the TCR T cells recognize an antigen on a hematological malignancy (e.g., CMV, WT1, HA-1). In certain embodiments, the TCR T cells recognize an antigen on a solid tumors (e.g., HBV, p53, mutant KRAS). In certain embodiments, the TCR T cells recognize SL9 associated with HIV.

As provided herein, the methods for producing mitochondria replaced T cells from exhausted T cells described in the present disclosure, are also applicable to producing mitochondria replaced T cells from senescent T cells. Senescent cells generally exhibit one or more phenotypic marker, such as increased secretion of inflammatory cytokines (e.g., interferon gamma (IFNγ) and/or tumor necrosis factor alpha (TNFα), growth factors, and proteases, as well as reduced and/or slower rates of cell population doublings, shortened telomeres, increased DNA damage response (DDR), or a combination thereof.

In some embodiments, the mitochondria replaced T cells generated according to the methods provided herein are produced from a senescent cell and the mitochondria replaced T cells have a decrease in senescence, as compared to a senescent T cell that has not been incubated with isolated exogenous mitochondria. In certain embodiments, the mitochondria replaced T cells generated according to the methods provided herein are produced from a senescent cell and have a decrease in secretion of inflammatory cytokines (e.g., interferon gamma (IFNγ) and/or tumor necrosis factor alpha (TNFα)). In certain embodiments, the mitochondria replaced T cells generated according to the methods provided herein are produced from a senescent cell and have a decrease in secretion of growth factors. In certain embodiments, the mitochondria replaced T cells generated according to the methods provided herein are produced from a senescent cell and have a decrease in secretion of proteases. In certain embodiments, the mitochondria replaced T cells generated according to the methods provided herein are produced from a senescent cell and have an increase in proliferation. In certain embodiments, the mitochondria replaced T cells generated according to the methods provided herein are produced from a senescent cell and have a decrease in telomere shortening. In certain embodiments, the mitochondria replaced T cells generated according to the methods provided herein are produced from a senescent cell and have a decrease in DNA damage response (DDR).

In some embodiments, the mitochondria replaced T cells generated according to the methods provided herein are produced from an exhausted T cell and the mitochondria replaced T cells have a decrease in senescence, as compared to an exhausted T cells that has not been incubated with isolated exogenous mitochondria. In certain embodiments, the mitochondria replaced T cells generated according to the methods provided herein are produced from an exhausted T and have a decrease in secretion of inflammatory cytokines (e.g., interferon gamma (IFNγ) and/or tumor necrosis factor alpha (TNFα)). In certain embodiments, the mitochondria replaced T cells generated according to the methods provided herein are produced from an exhausted T cell and have a decrease in secretion of growth factors. In certain embodiments, the mitochondria replaced T cells generated according to the methods provided herein are produced from an exhausted T cell and have a decrease in secretion of proteases. In certain embodiments, the mitochondria replaced T cells generated according to the methods provided herein are produced from an exhausted T cell and have an increase in proliferation. In certain embodiments, the mitochondria replaced T cells generated according to the methods provided herein are produced from an exhausted T cell and have a decrease in telomere shortening. In certain embodiments, the mitochondria replaced T cells generated according to the methods provided herein are produced from an exhausted T cell and have a decrease in DNA damage response (DDR).

In a specific embodiment, provided herein is a method for generating mitochondria replaced T cells using the method described in Example 8.1.

### 6.2 Methods of Treatment

As provided herein, the mitochondria replaced T cells generated according to the methods of the present disclosure are suitable for use as a cell-based therapy or therapies, such as in the methods described in Section 6.2.1, Section 6.2.2 and Section 6.2.3. For example, in some embodiments, an effective amount of the mitochondria replaced T cells generated according to the methods described in Section 6.1, can be combined with a pharmaceutically acceptable carrier to result in a pharmaceutical composition.

In some embodiments, provided herein is a composition (e.g., a pharmaceutical composition) comprising mitochondria replaced T cells generated according to the methods described herein. In some embodiments, provided herein is a composition (e.g., a pharmaceutical composition) comprising an effective amount of the mitochondria replaced T cells generated according to the methods described herein and a pharmaceutically acceptable carrier.

As used herein, the term “pharmaceutically acceptable” when used in reference to a carrier, is intended to mean that the carrier, diluent or excipient is not toxic or otherwise undesirable, (i.e., the material may be administered to a subject without causing any undesirable biological effects), and it is compatible with the other ingredients of the formulation. The term “carrier” refers to a diluent, adjuvant, excipient, or vehicle with which the therapeutic is administered. Such pharmaceutical carriers can be sterile liquids, such as saline solutions. A saline solution can be a carrier when the pharmaceutical composition is administered intravenously. Saline solutions and aqueous dextrose and glycerol solutions can also be employed as liquid carriers, particularly for injectable solutions. The composition, if desired, can also contain minor amounts of wetting or emulsifying agents, or pH buffering agents.

In certain embodiments, the effective amount of the mitochondria replaced T cells is about 1×10⁶ to about 1×10⁷ cells. In some embodiments, the effective amount of the mitochondria replaced T cells is about 10×10⁶ to about 900×10⁶ cells. In some embodiments, the effective amount of the mitochondria replaced T cells is about 50×10⁶ to about 800×10⁶ cells. In some embodiments, the effective amount of the mitochondria replaced T cells is about 100×10⁶ to about 700×10⁶ cells. In some embodiments, the effective amount of the mitochondria replaced T cells is about 200×10⁶ to about 900×10⁶ cells. In some embodiments, the effective amount of the mitochondria replaced T cells is about 250×10⁶ to 750×10⁶ cells. In some embodiments, the effective amount of the mitochondria replaced T cells is about 50×10⁶ cells. In some embodiments, the effective amount of the mitochondria replaced T cells is about 150×10⁶ cells. In some embodiments, the effective amount of the mitochondria replaced T cells is about 300×10⁶ cells. In some embodiments, the effective amount of the mitochondria replaced T cells is about 450×10⁶ cells. In some embodiments, the effective amount of the mitochondria replaced T cells is about 600×10⁶ cells. In some embodiments, the effective amount of the mitochondria replaced T cells is about 850×10⁶ cells.

In particular embodiments, the effective amount of the effective amount of the mitochondria replaced T cells is determined empirically, such as, for example, based on the weight of the subject, or the burden of the disease or disorder. In some embodiments, the effective amount of the mitochondria replaced T cell is about 1.0×10⁶ cells/kg to about 1.0×10⁷ cells/kg. In some embodiments, the effective amount of the mitochondria replaced T cell is about 1.0×10⁶ cells/kg to about 500×10⁶ cells/kg. In some embodiments, the effective amount of the mitochondria replaced T cell is about 1.0×10⁶ cells/kg to about 50×10⁶ cells/kg. In some embodiments, the effective amount of the mitochondria replaced T cell is about 1.0×10⁶ cells/kg to about 10×10⁶ cells/kg. In some embodiments, the effective amount of the mitochondria replaced T cell is about 1.0×10⁶ cells/kg to about 5.0×10⁶ cells/kg. In some embodiments, the effective amount of the mitochondria replaced T cell is about 10×10⁶ cells/kg to about 600×10⁶ cells/kg. In some embodiments, the effective amount of the mitochondria replaced T cell is about 50×10⁶ cells/kg to about 750×10⁶ cells/kg. In some embodiments, the effective amount of the mitochondria replaced T cells is about 1.0×10⁶ cells/kg. In some embodiments, the effective amount of the mitochondria replaced T cells is about 2.5×10⁶ cells/kg. In some embodiments, the effective amount of the mitochondria replaced T cells is about 5.0×10⁶ cells/kg. In some embodiments, the effective amount of the mitochondria replaced T cells is about 10.0×10⁶ cells/kg. In some embodiments, the effective amount of the mitochondria replaced T cells is about 50.0×10⁶ cells/kg. In some embodiments, the effective amount of the mitochondria replaced T cells is about 250.0×10⁶ cells/kg. In some embodiments, the effective amount of the mitochondria replaced T cells is about 500×10⁶ cells/kg.

In certain embodiments, any T cell therapy with therapeutic application that is susceptible to T cell exhaustion can benefit from mitochondria replacement. For example, in certain embodiments, the mitochondria replaced T cells provided herein, such as the mitochondria replaced T cells generated according to the methods described in Section 6.1 or in the Examples described infra, are suitable for use in treating a condition in which T cell exhaustion is caused by cancer. In certain embodiments, the mitochondria replaced T cells provided herein, such as the mitochondria replaced T cells generated according to the methods described in Section 6.1 or in the Examples described infra, are suitable for use in treating a condition in which T cell exhaustion is caused by a viral infection. In certain embodiments, the mitochondria replaced T cells provided herein, such as the mitochondria replaced T cells generated according to the methods described in Section 6.1 or in the Examples described infra, are suitable for use in treating a condition in which T cell exhaustion is caused by a bacterial infection. In certain embodiments, the mitochondria replaced T cells provided herein, such as the mitochondria replaced T cells generated according to the methods described in Section 6.1 or in the Examples described infra, are suitable for use in treating a condition in which T cell exhaustion is caused by a fungal infection. In certain embodiments, the mitochondria replaced T cells provided herein, such as the mitochondria replaced T cells generated according to the methods described in Section 6.1 or in the Examples described infra, are suitable for use in treating a condition in which T cell exhaustion is caused by obesity or metabolic disorder. In certain embodiments, the mitochondria replaced T cells provided herein, such as the mitochondria replaced T cells generated according to the methods described in Section 6.1 or in the Examples described infra, are suitable for use in treating a condition in which T cell exhaustion is caused alcoholism. In certain embodiments, the mitochondria replaced T cells provided herein, such as the mitochondria replaced T cells generated according to the methods described in Section 6.1 or in the Examples described infra, are suitable for use in treating a condition in which T cell exhaustion is caused by hypermotility. In certain embodiments, the mitochondria replaced T cells provided herein, such as the mitochondria replaced T cells generated according to the methods described in Section 6.1 or in the Examples described infra, are suitable for use in treating a condition in which T cell exhaustion is caused by excessive mental stress. In certain embodiments, the mitochondria replaced T cells provided herein, such as the mitochondria replaced T cells generated according to the methods described in Section 6.1 or in the Examples described infra, are suitable for use in treating a condition in which T cell exhaustion is caused by hypoxia. In certain embodiments, the mitochondria replaced T cells provided herein, such as the mitochondria replaced T cells generated according to the methods described in Section 6.1 or in the Examples described infra, are suitable for use in treating a condition in which T cell exhaustion is caused by injury.

In certain embodiments, the mitochondria replaced T cells provided herein, such as the mitochondria replaced T cells generated according to the methods described in Section 6.1 or in the Examples described infra, are suitable for use in treating a condition in which T cell senescence is caused by aging.

In certain embodiments, the mitochondria replaced T cells provided herein, such as the mitochondria replaced T cells generated according to the methods described in Section 6.1 or in the Examples described infra, are suitable for use in treating an aging-related immunological dysfunction.

In certain embodiments, the mitochondria replaced T cells provided herein, such as the mitochondria replaced T cells generated according to the methods described in Section 6.1 or in the Examples described infra, are suitable for use in treating a disease condition in which CD8+ T cell dysfunction is observed. In certain embodiments, the mitochondria replaced T cells provided herein, such as the mitochondria replaced T cells generated according to the methods described in Section 6.1 or in the Examples described infra, are suitable for use in treating a disease condition in which CD4+ T cell dysfunction is observed. In certain embodiments, the mitochondria replaced T cells provided herein, such as the mitochondria replaced T cells generated according to the methods described in Section 6.1 or in the Examples described infra, are suitable for use in treating a disease condition in which dysfunction in T cell priming is observed. In certain embodiments, the mitochondria replaced T cells provided herein, such as the mitochondria replaced T cells generated according to the methods described in Section 6.1 or in the Examples described infra, are suitable for use in treating a disease condition in which memory T cell dysfunction is observed. In certain embodiments, the mitochondria replaced T cells provided herein, such as the mitochondria replaced T cells generated according to the methods described in Section 6.1 or in the Examples described infra, are suitable for use in treating a disease condition in which B cell dysfunction is observed. In certain embodiments, the mitochondria replaced T cells provided herein, such as the mitochondria replaced T cells generated according to the methods described in Section 6.1 or in the Examples described infra, are suitable for use in treating a disease condition in which dysfunction in B cell priming is observed. In certain embodiments, the mitochondria replaced T cells provided herein, such as the mitochondria replaced T cells generated according to the methods described in Section 6.1 or in the Examples described infra, are suitable for use in treating a disease condition in which memory B cell dysfunction is observed. In certain embodiments, the mitochondria replaced T cells provided herein, such as the mitochondria replaced T cells generated according to the methods described in Section 6.1 or in the Examples described infra, are suitable for use in treating a disease condition in which innate lymphoid cell dysfunction is observed. In certain embodiments, the mitochondria replaced T cells provided herein, such as the mitochondria replaced T cells generated according to the methods described in Section 6.1 or in the Examples described infra, are suitable for use in treating a disease condition in which innate T cell dysfunction is observed. In certain embodiments, the mitochondria replaced T cells provided herein, such as the mitochondria replaced T cells generated according to the methods described in Section 6.1 or in the Examples described infra, are suitable for use in treating a disease condition in which innate B cell dysfunction is observed. In certain embodiments, the mitochondria replaced T cells provided herein, such as the mitochondria replaced T cells generated according to the methods described in Section 6.1 or in the Examples described infra, are suitable for use in treating a disease condition in which CD8+ T cell dysfunction is observed.

In certain embodiments, the mitochondria replaced T cells provided herein, such as the mitochondria replaced T cells generated according to the methods described in Section 6.1 or in the Examples described infra, are suitable for use in treating a disease condition in a cancer patient with T-cell exhaustion. For example, the expression of PD-L1 and/or PD-L2, the ligands of PD-1, is correlated with prognosis in some human malignancies, such as for example, esophageal cancer, hepatocellular carcinoma, soft tissue sarcoma, non-small cell lung cancer, breast cancer, ovarian cancer, melanoma, pancreatic cancer, cervical cancer, and colon cancer. In certain embodiments, the mitochondria replaced T cells provided herein, such as the mitochondria replaced T cells generated according to the methods described in Section 6.1 or in the Examples described infra, are suitable for use in treating a solid cancer patient with a lack or reduced T-cell infiltration into the tumor is observed. In certain embodiments, the mitochondria replaced T cells provided herein, such as the mitochondria replaced T cells generated according to the methods described in Section 6.1 or in the Examples described infra, are suitable for use in treating a cancer patient with the T-cells showing either T-cell exhaustion or senescence phenotype.

Anti-cancer therapy can promote tissue dysfunction and the early onset of various age-related symptoms in the treated cancer patient (See Wang B, et al., Trends Cancer. 2020 October; 6(10):838-857). Thus, in some embodiments, the mitochondria replaced T cells provided herein, such as the mitochondria replaced T cells generated according to the methods described in Section 6.1 or in the Examples described infra, are suitable for use in treating accumulation and persistence of anti-cancer therapy-induced senescent cells.

In certain embodiments, the mitochondria replaced T cells provided herein, such as the mitochondria replaced T cells generated according to the methods described in Section 6.1 or in the Examples described infra, are suitable for use in treating a fibrotic disease in which T-cell exhaustion or T-cell senescence is observed. In certain embodiments, the mitochondria replaced T cells provided herein, such as the mitochondria replaced T cells generated according to the methods described in Section 6.1 or in the Examples described infra, are suitable for use in treating macular disease in which T-cell exhaustion or T-cell senescence is observed. In certain embodiments, the mitochondria replaced T cells provided herein, such as the mitochondria replaced T cells generated according to the methods described in Section 6.1 or in the Examples described infra, are suitable for use in treating muscular degenerative disease in which T-cell exhaustion or T-cell senescence is observed. In certain embodiments, the mitochondria replaced T cells provided herein, such as the mitochondria replaced T cells generated according to the methods described in Section 6.1 or in the Examples described infra, are suitable for use in treating neuro degenerative disease in which T-cell exhaustion or T-cell senescence is observed.

In certain embodiments, treatment with mitochondria replaced T cells results one, two, or more, or all of the following: (1) a reduction in severity, progression, spread, and/or frequency of one or more symptoms, (2) elimination of one or more symptoms and/or underlying cause, (3) prevention of the occurrence of one or more symptoms and/or their underlying cause, and (4) improvement or remediation of damage. In specific embodiments, treatment includes therapeutic treatment as well as prophylactic, or suppressive measures for the condition, disease or disorder.

In some embodiments, the mitochondria replaced T cells for use as cell-based therapy or therapies, such as in the methods described in Section 6.2.1, Section 6.2.2 and Section 6.2.3, are autologous or allogeneic to the subject receiving administration of the mitochondria replaced T cells. In some embodiments, the mitochondria replaced T cells for use as a cell-based therapies, such as in the methods described in Section 6.2.1, Section 6.2.2 and Section 6.2.3, are autologous to the subject receiving administration of the mitochondria replaced T cells. In some embodiments, the mitochondria replaced T cells for use as a cell-based therapies, such as in the methods described in Section 6.2.1, Section 6.2.2 and Section 6.2.3, are allogeneic to the subject receiving administration of the mitochondria replaced T cells.

### 6.2.1 Methods of Treating an Age-Related Disease

In one aspect, provided herein is a method for treating or ameliorating a symptom of an age-related disease, comprising administering to the subject a composition that includes an effective amount of the mitochondria replaced T cells generated according to the methods described in Section 6.1, and a pharmaceutically acceptable carrier. In some embodiments of the methods provided herein, the age-related disease is selected from the group consisting of an autoimmune disease, or cancer. In a specific embodiment, the subject is human.

In certain embodiments, the method for ameliorating a symptom of an age-related disease (e.g., cancer or an autoimmune disease) includes one, two or more, or all of the following: (1) a reduction in severity, progression, spread, and/or frequency of one or more symptoms, (2) elimination of one or more symptoms and/or underlying cause, (3) prevention of the occurrence of one or more symptoms and/or their underlying cause, and (4) improvement or remediation of damage. In one embodiment, the method for ameliorating a symptom of an age-related disease (e.g., cancer or an autoimmune disease) includes a reduction the severity of the symptom. In one embodiment, the method for ameliorating a symptom of an age-related disease (e.g., cancer or an autoimmune disease) includes a reduction in the progression of the symptom. In another embodiment, the method for ameliorating a symptom of an age-related disease (e.g., cancer or an autoimmune disease) includes a reduction in the spread of the symptom. In another embodiment, the method for ameliorating a symptom of an age-related disease (e.g., cancer or an autoimmune disease) reduces the frequency of the symptom. In another embodiment, the method for ameliorating a symptom of an age-related disease (e.g., cancer or an autoimmune disease) includes an elimination of the symptom. In another embodiment, the method for ameliorating a symptom of an age-related disease (e.g., cancer or an autoimmune disease) comprises prevention of the occurrence of symptoms of the age-related disease (e.g., cancer or an autoimmune disease). In another embodiment, the method for ameliorating a symptom of an age-related disease (e.g., cancer or an autoimmune disease) comprises improvement of damage from the age-related disease (e.g., cancer or an autoimmune disease). In another embodiment, the method for ameliorating a symptom of an age-related disease (e.g., cancer or an autoimmune disease) comprises a remediation of damage from the age-related disease (e.g., cancer or an autoimmune disease).

In a specific embodiment, provided herein is a method for treating a cancer in a subject in need thereof, comprising administering to the subject a composition that includes an effective amount of the mitochondria replaced T cells generated according to the methods described herein (e.g., in Section 6.1 or the Example), and a pharmaceutically acceptable carrier. In another specific embodiment, provided herein is a method for ameliorating a symptom of a cancer in a subject in need thereof, comprising administering to the subject a composition that includes an effective amount of the mitochondria replaced T cells generated according to the methods described herein (e.g., in Section 6.1 or the Example), and a pharmaceutically acceptable carrier. In particular embodiments, the exhausted T cells and mitochondria replaced T cells comprise an exogenous polynucleotide encoding a T cell receptor (TCR) or a chimeric antigen receptor (CAR). In certain embodiments, the exhausted T cells and mitochondria replaced T cells have been genetically modified to express a T cell receptor (TCR) or a chimeric antigen receptor (CAR). In other embodiments, the mitochondria replaced T cells have been genetically modified to express a T cell receptor (TCR) or a chimeric antigen receptor (CAR).

In another specific embodiment, provided herein is a method for treating accumulation and persistence of anti-cancer therapy-induced senescent cells in a subject in need thereof, comprising administering to the subject a composition that includes an effective amount of the mitochondria replaced T cells generated according to the methods described herein (e.g., in Section 6.1 or the Example), and a pharmaceutically acceptable carrier. In another specific embodiment, provided herein is a method for ameliorating a symptom of an anti-cancer therapy-induced senescent cell in a subject in need thereof, comprising administering to the subject a composition that includes an effective amount of the mitochondria replaced T cells generated according to the methods described herein (e.g., in Section 6.1 or the Example), and a pharmaceutically acceptable carrier.

### 6.2.2 Methods of Treating a Chronic Infection

T-cell exhaustion has also been reported in various human chronic viral infections such as hum an immunodeficiency virus (HIV), hepatitis B (HBV), and hepatitis C (HCV), as well as non-viral chronic infections such as malaria and Mycobacterium tuberculosis. Thus, in one aspect, provided herein is a method for ameliorating a symptom of a chronic infection in a subject in need thereof, comprising administering to the subject a composition that includes an effective amount of the mitochondria replaced T cells generated according to the methods described herein (e.g., in Section 6.1 or the Example), and a pharmaceutically acceptable carrier.

In certain embodiments, the chronic infection is a viral infection. Non-limiting examples of viral infections suitable for treatment with the mitochondria replaced T cells described herein include a human immunodeficiency virus (HIV) infection, a hepatitis B (HBV) infection, hepatitis C (HCV) infection, a cytomegalovirus infection, and a SARS-COV-2 infection. In one embodiment, the chronic viral infection is HIV. In one embodiment, the chronic viral infection is a HBV infection. In another embodiment, the chronic viral infection is a HCV infection. In another embodiment, the chronic viral infection is a cytomegalovirus infection. In one embodiment, the chronic viral infection is SARS-COV-2 infection. In other embodiments, the chronic infection is a non-viral infection. For example, in certain embodiments, the chronic infection is a bacterial or fungal infection.

In certain embodiments, the method for ameliorating a symptom of a chronic infection includes one, two, or more, or all of the following: (1) a reduction in severity, progression, spread, and/or frequency of one or more symptoms, (2) elimination of one or more symptoms and/or underlying cause, (3) prevention of the occurrence of one or more symptoms and/or their underlying cause, and (4) improvement or remediation of damage. In one embodiment, the method for ameliorating a symptom of a chronic infection includes a reduction the severity of the symptom. In one embodiment, the method for ameliorating a symptom of a chronic infection includes a reduction in the progression of the symptom. In another embodiment, the method for ameliorating a symptom of a chronic infection includes a reduction in the spread of the symptom. In another embodiment, the method for ameliorating a symptom of a chronic infection reduces the frequency of the symptom. In another embodiment, the method for ameliorating a symptom of a chronic infection includes an elimination of the symptom. In another embodiment, the method for ameliorating a symptom of a chronic infection comprises prevention of the occurrence of symptoms of the chronic infection. In another embodiment, the method for ameliorating a symptom of a chronic infection comprises improvement of damage from the chronic infection. In another embodiment, the method for ameliorating a symptom of a chronic infection comprises a remediation of damage from the chronic infection.

### 6.2.3 Methods of Treating a Mitochondrial Disease or Disorder

In another aspect, provided herein is a method for ameliorating a symptom of mitochondrial complex III deficiency to a subject in need thereof, comprising administering to the subject a composition that includes an effective amount of the mitochondria replaced T cells generated according to the methods described herein (e.g., in Section 6.1 or the Example), and a pharmaceutically acceptable carrier.

Mitochondrial complex III is essential for regulatory T cell (Treg) suppressive function. For example, it has been shown that Treg cells require mitochondrial complex III to maintain immune regulatory gene expression and suppressive function (see Weinberg, S. et al. Nature vol. 565, 7740 (2019): 495-499). Mitochondrial complex III deficiency is a genetic condition. It is generally caused by mutations in nuclear DNA in the BCSIL, UQCRB and UQCRQ genes and inherited in an autosomal recessive manner. However, it may also be caused by mutations in mitochondrial DNA in the MTCYB gene, which is passed down maternally or occurs sporadically and may result in a milder form of the condition.

Accordingly, in some embodiments, the mitochondria replaced T cells are Treg cells and the mitochondria replaced T cells are administered to a subject having mitochondrial complex III deficiency. In specific embodiments, the subject is a human subject.

In certain embodiments, the method for ameliorating a symptom of mitochondrial complex III deficiency includes one, two, three, or more, or all of the following: (1) a reduction in severity, progression, spread, and/or frequency of one or more symptoms, (2) elimination of one or more symptoms and/or underlying cause, (3) prevention of the occurrence of one or more symptoms and/or their underlying cause, and (4) improvement or remediation of damage. In one embodiment, the method for ameliorating a symptom of mitochondrial complex III deficiency includes a reduction the severity of the symptom. In one embodiment, the method for ameliorating a symptom of mitochondrial complex III deficiency includes a reduction in the progression of the symptom. In another embodiment, the method for ameliorating a symptom of mitochondrial complex III deficiency includes a reduction in the spread of the symptom. In another embodiment, the method for ameliorating a symptom of mitochondrial complex III deficiency reduces the frequency of the symptom. In another embodiment, the method for ameliorating a symptom of mitochondrial complex III deficiency includes an elimination of the symptom. In another embodiment, the method for ameliorating a symptom of mitochondrial complex III deficiency comprises prevention of the occurrence of symptoms of the mitochondrial complex III deficiency. In another embodiment, the method for ameliorating a symptom of mitochondrial complex III deficiency comprises improvement of damage from the mitochondrial complex III deficiency. In another embodiment, the method for ameliorating a symptom of mitochondrial complex III deficiency comprises a remediation of damage from the mitochondrial complex III deficiency.

### 6.3 Biological Assays

In specific embodiments, successful generation of mitochondria replaced T cells from exhausted T cells results in T cells that have improved effector function, relative to the exhausted T cells without mitochondria replacement. Various functional assays can be used to assess and evaluate phenotype of the mitochondria replaced T cells.

In some embodiments, the mitochondria replaced T cells have improved mitochondrial function, relative to T cells without mitochondria replacement. A person skilled in the art would understand how to evaluate mitochondrial function. For example, cell-based assays, such as the Seahorse Bioscience XF Extracellular Flux Analyzer, can used performed for the determination of basal oxygen consumption, glycolysis rates, ATP production, and respiratory capacity to assess mitochondrial dysfunction. Similarly, the Oroboros 02K respirometer can also be used to establish quantitative functional mitochondrial diagnosis. It is understood that the assay examples described above are exemplary and are not inclusive of all methods to evaluate mitochondrial function.

Increased cell proliferation can also be an indicator of improved T cell function. An exemplary assay for measuring cell proliferation of T cells is a mixed lymphocyte reaction (MLR) assay. The MLR assay generally involves combining a population of the mitochondria replaced T cells, such as CD4+ T cells, with a different population of lymphocytes and measuring proliferation. In some embodiments, the mitochondria replaced T cells generated according to the methods provided herein have increased cell proliferation, as compared to an exhausted T cell.

Another exemplary assay that can be used to assess the function of the mitochondria replace cells, such as in cytotoxic T cells, is a cytotoxic T cell (CTL) assay. A CTL assay indicates the presence and cytotoxic activity of T cells to a specific antigen and allows to examine the influence of a test item on this immune function. Accordingly, in some embodiments, the mitochondria replaced T cells generated according to the methods provided herein have increased CTL response, as compared to an exhausted T cell.

Ca2+ signaling is critical to T cell activation as a means of rapidly activating and integrating numerous signaling pathways to generate widespread changes in gene expression and function. Various assays to assess Ca2+ signaling are known in the art (See Samakai E, et al., Signaling Mechanisms Regulating T Cell Diversity and Function. Boca Raton (FL): CRC Press/Taylor & Francis; 2018. Chapter 10.) Thus, in some embodiments, the mitochondria replaced T cells generated according to the methods provided herein have increased Ca2+ signaling, as compared to exhausted T cells.

Telomere length can also serve as an indicator of mitochondria replaced cell's function. Telomere length can be assessed using any method known in the art. One exemplary technique is by measuring absolute telomere length by qPCR. Thus, in some embodiments, the mitochondria replaced T cells generated according to the methods provided herein have a decreased telomere shortening, as compared to exhausted T cells.

In some embodiments the mitochondria replaced cells exhibits a reduction in T cell exhaustion. FACS analysis for exhaustion markers (e.g., PD-1/TIM3/LAG3) can be used to assess T cell exhaustion. Accordingly, in some embodiments, the mitochondria replaced T cells generated according to the methods provided herein have a reduction in exhaustion, as compared to exhausted T cells.

As provided herein, in some embodiments the T cells that are used to generate a mitochondria replaced cell are senescent and the mitochondria replaced cell exhibits a decrease in senescence. Thus, assessing Senescence Associated Secretory Phenotype (SASP) can serve as a functional assay. SASP includes increased secretion of inflammatory cytokines (e.g., interferon gamma (IFNγ) and/or tumor necrosis factor alpha (TNFα), growth factors, and proteases, as well as reduced and/or slower rates of cell population doublings, shortened telomeres, increased DNA damage response (DDR), or a combination thereof. FACS analysis for senescence markers (e.g., CD57/KIR/KLRG1) can also be employed. Accordingly, in some embodiments, the mitochondria replaced T cells generated according to the methods provided herein have a decrease in senescence, as compared to senescent T cells.

### 6.4 Nucleotide and Protein Detection Assays

Techniques known to one of skill in the art or described herein (e.g., in the Example) may be used to assess the amount of the nucleotide encoding XbaIR (e.g., PCR or qPCR), or the amount of the XbaIR or the fusion protein (e.g., immunoblot, ELISA).

### 7. EMBODIMENTS

This invention provides the following non-limiting embodiments.

A1. A method for producing mitochondria replaced T cells from exhausted T cells, the method comprising:
incubating exhausted T cells having reduced endogenous mitochondria DNA (mtDNA) copy number with isolated exogenous mitochondria for a sufficient period of time to generate mitochondria replaced T cells in which expression of programmed cell death-1 (PD-1) is decreased by at least 1.1 fold relative to the expression of PD-1 by the exhausted T cells from which the mitochondria replaced T cells were produced,
wherein the mitochondria replaced T cells have improved effector function relative to the exhausted T cells.

A2. A method for producing mitochondria replaced T cells from exhausted T cells, the method comprising:
(a) electroporating exhausted T cells with a nucleic acid sequence comprising a nucleotide sequence encoding a fusion protein comprising a mitochondrial-targeted sequence (MTS) and XbaIR to reduce endogenous mitochondrial DNA (mtDNA) copy number; and
(b) incubating the exhausted T cells having reduced endogenous mitochondria DNA (mtDNA) copy number with isolated exogenous mitochondria for a sufficient period of time to generate a mitochondria replaced T cell in which expression of PD-1 is decreased by at least 1.1 fold relative to the expression of PD-1 by the exhausted T cells from which the mitochondria replaced T cells were produced, wherein the mitochondria replaced T cells have improved effector function relative to the exhausted T cells.

A3. The method of embodiment A1 or A2, wherein the incubation of the exhausted T cells with the isolated exogenous mitochondria occurs in the presence of rapamycin.

A4. The method of embodiment A3, wherein rapamycin is present at a concentration of 100 nM to 1000 nM.

A5. The method of any one of embodiments A1 to A4, wherein the expression of PD-1 is decreased by at least 1.2 fold.

A6. The method of any one of embodiments A1 to A4, wherein the expression of PD-1 is decreased by at least 1.25 fold.

A7. The method of any one of embodiments A1 to A4, wherein the expression of PD-1 is decreased by at least 1.5 fold.

A8. The method of any one of embodiments A1 to A4, wherein the expression of PD-1 is decreased by at least 2 fold.

A9. The method of any one of embodiments A1 to A4, wherein the expression of PD-1 is decreased by at least 5 fold.

A10. The method of any one of embodiments A1 to A4, wherein the expression of PD-1 is decreased by about 1.1 fold to about 1.5 fold.

A11. The method of any one of embodiments A1 to A10, wherein the method reduces the expression of T-cell immunoglobulin and mucin domain-containing protein 3 (TIM3), lymphocyte-activated gene-3 (LAG3), T Cell immunoglobulin and ITIM domain (TIGIT), TOX, or a combination thereof.

A12. The method of any one of embodiments A1 to A11, wherein the isolated exogenous mitochondria is about 20 μg to about 80 μg protein per 1×10⁶ cells.

A13. The method of any one of embodiments A1 to A12, wherein the mitochondria replaced T cells comprise at least 20% of the exogenous mtDNA.

A14. The method of any one of embodiments A1 to A12, wherein the mitochondria replaced T cells comprises at least 20% of exogenous mtDNA and no more than 80% exogenous mtDNA, as measured by TaqMan Single Nucleotide Polymorphism (SNP) Assay.

A15. The method of any one of embodiments A1 to A14, wherein the sufficient period of time to generate mitochondria replaced T cells is at least approximately 24 hours.

A16. The method of any one of embodiments A1 to A14, wherein the sufficient period of time to generate mitochondria replaced T cells is at least 36 hours.

A17. The method of any one of embodiments A1 to A14, wherein the sufficient period of time to generate mitochondria replaced T cells is at least 48 hours.

A18. The method of any one of embodiments A1 to A14, wherein the sufficient period of time to generate mitochondria replaced T cells is about 24 hours to about 72 hours.

A19. The method of any one of embodiments A1 to A18, wherein the improved effector function comprises increased proliferation, increased cytotoxicity, increased secretion of cytokines, or a combination thereof.

A20. The method of any one of embodiments A1 to A19, wherein the exhausted T cells comprise an exogenous polynucleotide encoding a T cell receptor (TCR) or a chimeric antigen receptor (CAR).

A21. The method of any one of embodiments A1 to A19, wherein the exhausted T cells have been genetically modified to express a T cell receptor (TCR) or a chimeric antigen receptor (CAR).

A22. A mitochondria replaced T cell generated by the method of any one of embodiments A1 to A19.

A23. A mitochondria replaced T cell generated by the method of embodiment A20 or A21.

A24. A composition comprising an effective amount of the mitochondria replaced T cell of embodiment A22, and a pharmaceutically acceptable carrier.

A25. A method for ameliorating a symptom of a chronic viral infection in a subject in need thereof, comprising administering to the subject the composition of embodiment A24.

A25. The method of embodiment A25, wherein the chronic viral infection is a human immunodeficiency virus (HIV) infection, a hepatitis B virus (HBV) infection, a cytomegalovirus infection (CMV), and a Severe Acute respiratory syndrome coronavirus (SARS-COV)-2 infection.

A27. A composition comprising an effective amount of the mitochondria replaced T cell of embodiment A23, and a pharmaceutically acceptable carrier.

A28. A method for treating a cancer in a subject in need thereof, comprising administering to the subject the composition of embodiment A24 or A27.

A29. A method for ameliorating a symptom of a cancer in a subject in need thereof, comprising administering to the subject the composition of embodiment A24 or A27.

A30. A method for treating a disease or condition associated with, involving, or caused by T cell exhaustion in a subject in need thereof, comprising administering to the subject the composition of embodiment A24 or A27, wherein the disease or condition is:
(a) cancer;
(b) a viral infection;
(c) a bacterial infection;
(d) obesity or a metabolic disorder;
(e) alcoholism;
(f) hypermotility;
(g) excessive mental stress;
(h) hypoxia;
(i) an injury;
(i) aging;
(k) aging related immunological dysfunction;
(l) a fibrotic disease;
(m) a macular disease;
(n) a muscular degenerative disease; or
(o) a neurodegenerative disease.

A31. A method for ameliorating a symptom of a disease or condition associated with, involving, or caused by T cell exhaustion in a subject in need thereof, comprising administering to the subject the composition of embodiment A24 or A27, wherein the disease or condition is:
(a) cancer;
(b) a viral infection;
(c) a bacterial infection;
(d) obesity or a metabolic disorder;
(e) alcoholism;
(f) hypermotility;
(g) excessive mental stress;
(h) hypoxia;
(i) an injury;
(j) aging;
(k) aging related immunological dysfunction;
(l) a fibrotic disease;
(m) a macular disease;
(n) a muscular degenerative disease; or
(o) a neurodegenerative disease.

A32. A method for treating a disease or condition associated with or involving, or caused by T cell exhaustion in a subject in need thereof, comprising administering to the subject the composition of embodiment A24 or A27, wherein the disease or condition is:
(a) CD8+ T cell dysfunction;
(b) CD4+ T cell dysfunction;
(c) dysfunction in T cell priming;
(d) memory T cell dysfunction;
(e) effector B cell dysfunction;
(f) dysfunction in B cell priming;
(g) memory B cell dysfunction;
(h) innate lymphoid cell dysfunction;
(i) innate T cell dysfunction; or
(j) innate B cell dysfunction.

A33. The method of any one of embodiments A25, A26, or A28 to A32, wherein the subject is human.

### 8. EXAMPLES

The examples in this section are offered by way of illustration, and not by way of limitation. The following examples are presented as exemplary embodiments of the invention. They should not be construed as limiting the broad scope of the invention.

### 8.1 Example 1: Methods and Compositions for Reducing Immune Cell Exhaustion Using Mitochondria Replacement

The following example demonstrates that the phenotypic markers of exhausted T cells can be reduced by using mitochondria replacement in the exhausted T cells to generate Mir T cells.

### 8.1.1 Materials & Methods

Isolation and Cell Culture of Primary Human T lymphocytes: Peripheral blood was kindly provided by a healthy person recruited by voluntary application under the approval from an ethical committee. Venous heparinized blood was taken according to the standard procedures from the median cubital vein (NP-EN0507, NIPRO, Osaka, Japan). Human peripheral blood mononuclear cells (PBMC) were isolated from human peripheral blood using density-gradient centrifugation with 1.077 g/ml Percoll (GE Healthcare Life Sciences, Buckinghamshire, England). Cells were cultured in TexMACS medium (Miltenyi Biotec) supplemented with 10% fetal bovine serum, 1% penicillin/streptomycin (Thermo Fisher Scientific incorporated), 20 μM of IL-7, and 10 μM of IL-15 on cell culture plates activated with anti-CD3 antibody and anti-CD28 antibody (Miltenyi Biotec). Cells were incubated at 37° C. in a humidified 5% CO₂ incubator.

Generation of CAR T cells: CAR T cells have been generated from T cells of peripheral blood, which were drawn from healthy volunteer, by transferring a recombinant vector carrying a ligand for ephedrine type B receptor 4 (EPHB4), which is expressed in rhabdomyosarcoma (see Kubo, H., et al. (2021). “Development of non-viral, ligand-dependent, EPHB4-specific chimeric antigen receptor T cells for treatment of rhabdomyosarcoma.” Mol Ther Oncolytics 20: 646-658).

Induction of Exhaustion of CAR T Cells: CAR T cells expressing the ligand for EPHB4 were co-cultivated with rhabdomyosarcoma cell expressing EPHB4, Rh30 cells, without any cytokine signaling. Following 3 days of co-culture, the CAR T cells significantly expressed PD-1 that is a marker for exhausted T cells. The environment where the CAR T is present with sustained antigen stimulation and without supporting signal (CD3, CD28, IL-7, and IL-15) mimics the microenvironment of solid tumors.

Mitochondrial Isolation and Transfer to Human T cells: Mitochondria were isolated from DsRed-Mt EMCs by differential centrifugation as described previously. In brief, the cells were harvested from culture dishes with homogenization buffer [HB; 20 mM HEPES-KOH (pH 7.4), 220 mM mannitol and 70 mM sucrose] containing a protease inhibitor mixture (Sigma-Aldrich, St. Louis, Missouri, USA). The cell pellet was resuspended in HB and incubated on ice for 5 min. The cells were ruptured by 10 strokes of a 27-gauge needle on ice. The homogenate was centrifuged (400×g, 4° C.; 5 min.) two times to remove unbroken cells. The mitochondria were harvested by centrifugation (6000×g, 4° C.; 5 min.) and resuspended in HB. The amounts of isolated mitochondria were expressed as protein concentration using a Bio-Rad protein assay kit (Bio-Rad Laboratories, incorporated, Richmond, CA, USA). On day 3 after exhausted CAR-T generation protocol, the cells received MTS-fused XbaIR to reduce mtDNA copy number by electroporation. Following the gene transfer, the cells are replated on CD3/CD28 coated dish in a complete T cell growth medium. Mitochondrial transfer was conducted by co-incubating the isolated mitochondria with the ρ(−) cells derived from the CAR-T following a centrifugation (1,500×g for 5 min at room temperature).

Direct sequencing to D-loop sequence on mitochondrial DNA: The whole DNAs were extracted from cells using NucleoSpin Tissue (MACHEREY-NAGEL GmbH & Co. KG). The extracted DNA was used as a template to amplify the D-loop sequence of mitochondrial DNA using GoTaq® Green Master Mix (Promega KK.) according to GoTaq® Green Master Mix Protocol. The primer sequences were forward primer: 5′-ctctgttctttcatggggaagc-3′ (SEQ ID NO:3) and reverse primer: 5′-cataaactgtggggggtgtct-3′ (SEQ ID NO:4). After amplification, the PCR products were electrophoresed on a 1% agarose gel, and the 1,134 bp band was purified using NucleoSpin Gel and PCR Clean-up kit (MACHEREY-NAGEL GmbH & Co. KG). The extracted PCR products were sequenced using the forward primer used for PCR amplification and Applied Biosystems 3730×l DNA analyzer (Thermo Fisher Scientific Inc.).

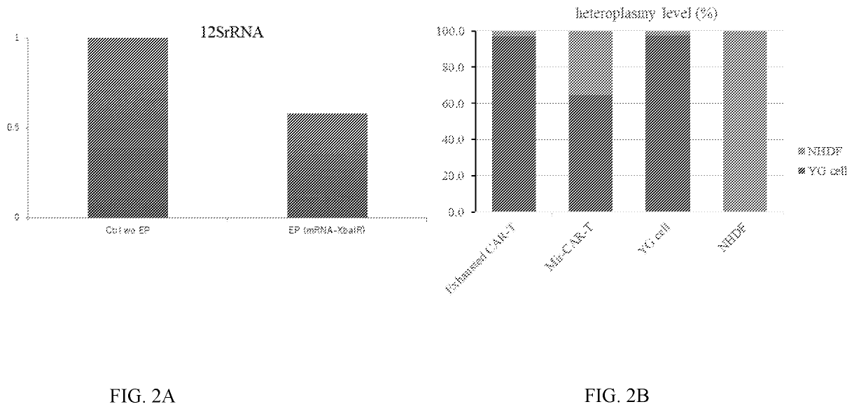

Heteroplasmy analysis to mtDNA using TaqMan Single Nucleotide Polymorphism (SNP) assay: To determine heteroplasmy ratios, wild-type (NHDF) and mutant (YG cell) allele-specific TaqMan probes for the TaqMan SNP assay were designed. The extracted DNA (10 ng) was used for quantitative PCR with the TaqMan Universal PCR Master Mix kit (Thermo Fisher Scientific Incorporated) on a CFX connect real-time system (Bio-Rad Laboratories, Incorporated) under the following conditions: 40 cycles of PCR (95° C. for 15 sec and 60° ° C. for 1 min) after initial denaturation (95° C. for 10 min). The primer sequences were 5′-TTACTGCCAGCCACCATGAA-3′ (SEQ ID NO:5) as the forward primer and 5′-TTGATGTGGATTGGGTTTTTATGT-3′ (SEQ ID NO:6) as the reverse primer. The probe for wild type (NHDF) was FAM-ACAGGTGGTTAAGTATT-MGB (SEQ ID NO:7), and the probe for the mutation (YG cell) was VIC-CAGGTGGTCAAGTAT-MGB (SEQ ID NO:8). A calibration curve was created using known copy numbers of plasmids containing the amplified mtDNA D-loop fragments for either wild-type or mutant sequences. Validation of the TaqMan SNP showed that YG cells were distinguishable from NHDF cells based on the mtDNA sequence (FIG. 2B).

Analysis of PD-1 expression in CAR-T cells: CAR-T cells (5×10⁶) were co-cultured with human rhabdomyosarcoma Rh-30 cells (1×10⁷) for 3 days to exhaustion. The cultured cells were washed with PBS (300×g for 5 min), suspended in 1×10⁵ in 200 μl MACS buffer (Miltenyi Biotec), and 5 μl each FITC anti-human CD279 (PD-1) Antibody (329904, BioLegend, Inc.) and PE anti-human CD3 Antibody (300408, BioLegend, Inc.), and 1 μl of 7-AAD (559925, Becton, Dickinson and Company) were added. The mixture was incubated at 37° C. for 30 min. After washing with the MACS buffer, the mixture was suspended in 500 μl of MACS buffer and analyzed for PD-1 expression on the cell surface using Cell Sorter MA900 (Sony). PD-1 and CD3 expression levels were analyzed using FlowJo software (Becton, Dickinson and Company).

Measurement of Mean Fluorescence Intensity using FlowJo software: Mean Fluorescence Intensity (MFI) was measured using the statistical function Geometric mean fluorescence intensity method of FlowJo software (version 10.6). Briefly, select the FITC group in the workspace and click on the Statistics function button. Then select the Geometric Mean and FITC parameters in the dialog and click the Add button. Then, the MFI values will be displayed in the workspace.

### 8.1.2 Results

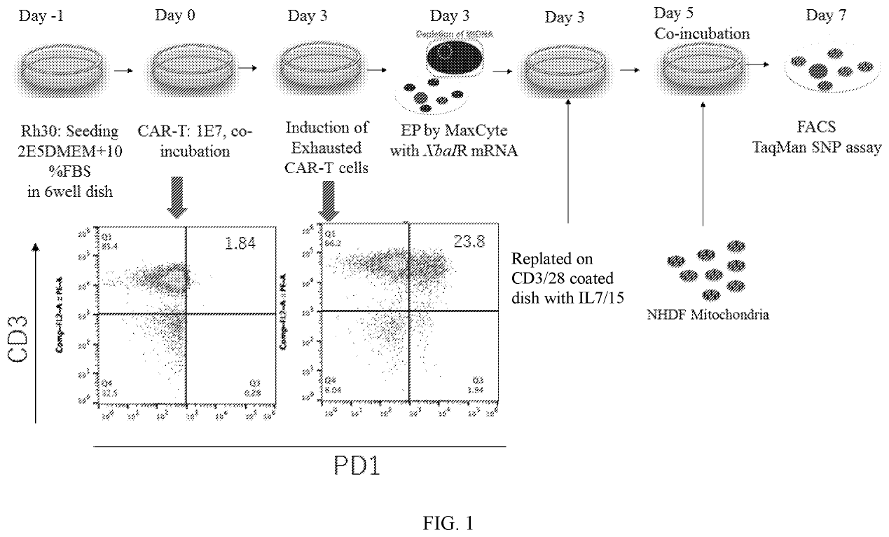

Rh30 human rhabdomyosarcoma (2×10⁵) cells were seeded in a 6-well dish and cultured in DMEM+10% FBS. The following day (“Day 0”), EPHB4-specific chimeric antigen receptor CAR-T cells (1×10⁷) were added to the same 6-well dish and co-incubated with the Rh30 cells for three days. FACS analysis of the expression levels of the exhaustion marker PD-1 by the CAR-T cells at Day 0 and Day 3 indicated that the percentage of CD3+/PD1+ cells increased from 1.84% to 23.8% after co-culture (FIG. 1). Thus, the co-culture generated exhausted CAR-T cells.

At day 3, endogenous mitochondrial DNA in the exhausted CAR-T cells was depleted by introducing XbaIR mRNA into the CAR-T cells by electroporation. As the negative control, the exhausted CAR-T cells received only electroporation without XbaIR mRNA and subjected to the same electroporation and the CAR-T cells contacted with XbaIR mRNA. The surrogate marker of mtDNA content, 12S rRNA, was halved on day 3, confirming that XbaIR caused effective mtDNA reduction (FIG. 2A).

Following, electroporation, the CAR-T cells were replated on a CD3/28 coated dish with IL-7/IL-15 and cultured for two days (FIG. 1). At day 5, normal human derived fibroblasts (NHDF)-derived donor mitochondria was co-cultured with the electroporated CAR-T cells that were contacted with or without XbaIR for two additional days to generate Mir CAR-T cells or the negative control exhausted CAR-T cells, respectively. FACS and TaqMan SNP assays were performed at day 7.

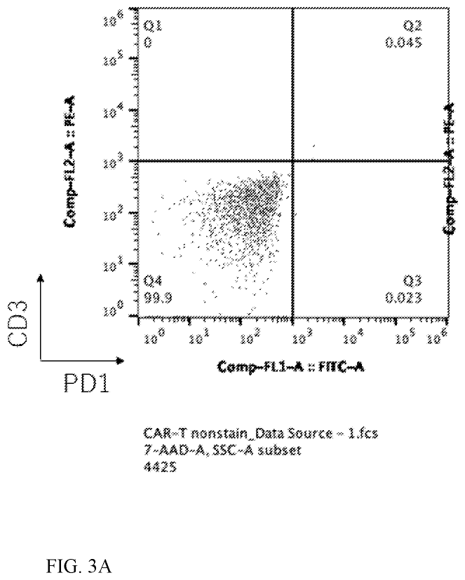

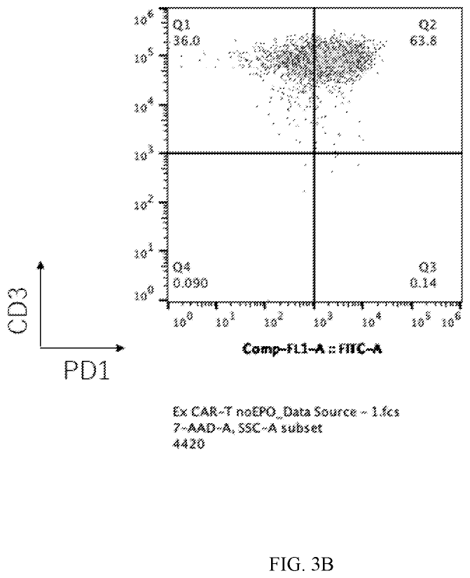

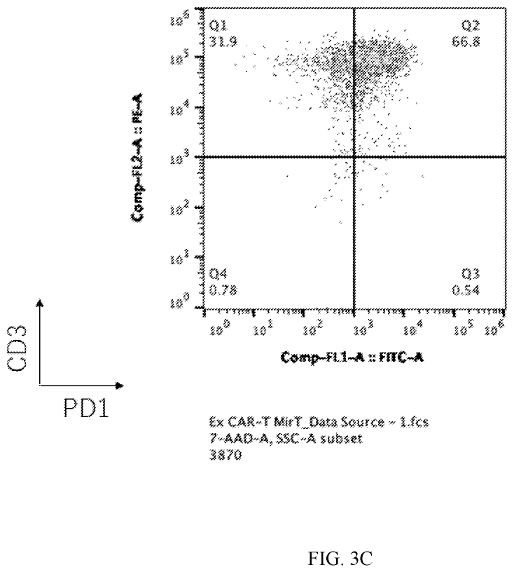

The results from the TaqMan SNP assay measured on day 7, indicated that Mir CAR-T cells contained about 40% NHDF-derived donor mtDNA (FIG. 2B). Moreover, FACS analysis on Day 7 indicated that the percentage of CD3+/PD-1+ cells was 0.045% for unstained cells (FIG. 3A), 63.8% for exhausted CAR-T cells that did not undergo mitochondrial depletion (FIG. 3B), and 66.8% for exhausted Mir CAR T cells (FIG. 3C).

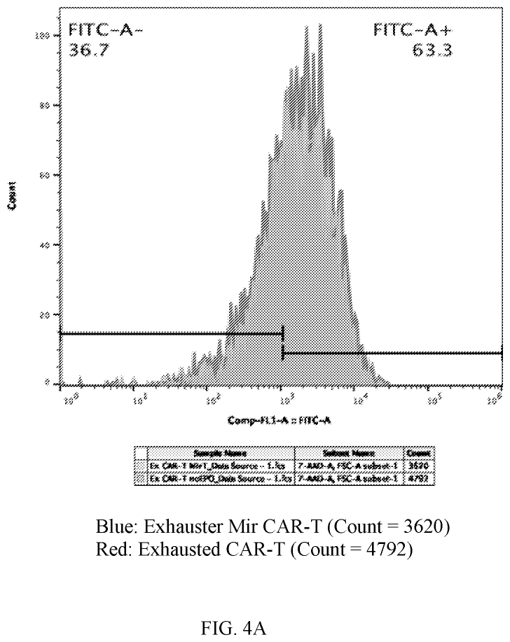

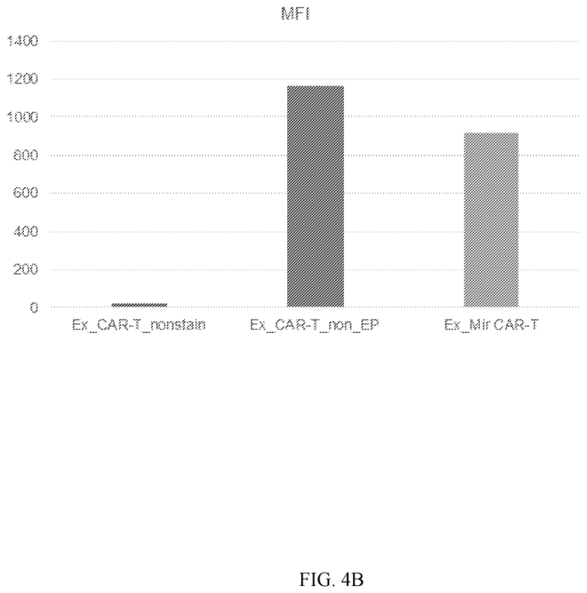

Expression analysis of PD-1 antigen after two days post-mitochondrial replacement revealed that PD-1 expression on the exhausted Mir CAR-T cells was decreased approximately 1.3 fold (or decreased by 24%) relative to exhausted CAR-T cells not electroporated with XbaIR (FIG. 4A and Table 1). Fold change was calculated by dividing the PD-1 antigen count in the exhausted CAR-T cells by the PD-1 antigen count in the exhausted Mir CAR-T cells. Percent change was calculated as follows: [[(PD-1 antigen count in the exhausted Mir CAR-T cells)—(PD-1 antigen count in the exhausted CAR-T cells)]÷PD-1 antigen count in the exhausted CAR-T cells]×100.

TABLE 1

| Cells | PD-1 antigen count |
|---|---|
| Exhausted CAR-T cells | 4792 |
| Exhausted Mir CAR-T cells | 3620 |

In addition, a bar graph of Mean Fluorescence Intensity shows that the intensity of PD-1 is decreased in Mir CAR-T cells, indicating that the expression of PD-1 antigen is decreased (FIG. 4B).

Taken together, these results demonstrate that mitochondrial replacement in exhausted T cells is able to reduce the expression of PD-1, an exemplary phenotypic marker of exhaustion. Such Mir T cells with reduced expression of PD-1 have utility in treating various conditions, such as cancer and chronic viral infection.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed is:

1. A method for producing mitochondria replaced T cells from exhausted T cells, the method comprising:
incubating exhausted T cells having reduced endogenous mitochondria DNA (mtDNA) copy number with isolated exogenous mitochondria for a sufficient period of time to generate mitochondria replaced T cells in which expression of programmed cell death-1 (PD-1) is decreased by at least 1.1 fold relative to the expression of PD-1 by the exhausted T cells from which the mitochondria replaced T cells were produced,
wherein the mitochondria replaced T cells have improved effector function relative to the exhausted T cells.

2. A method for producing mitochondria replaced T cells from exhausted T cells, the method comprising:
(a) electroporating exhausted T cells with a nucleic acid sequence comprising a nucleotide sequence encoding a fusion protein comprising a mitochondrial-targeted sequence (MTS) and XbaIR to reduce endogenous mitochondrial DNA (mtDNA) copy number; and
(b) incubating the exhausted T cells having reduced endogenous mitochondria DNA (mtDNA) copy number with isolated exogenous mitochondria for a sufficient period of time to generate a mitochondria replaced T cell in which expression of PD-1 is decreased by at least 1.1 fold relative to the expression of PD-1 by the exhausted T cells from which the mitochondria replaced T cells were produced, wherein the mitochondria replaced T cells have improved effector function relative to the exhausted T cells.

3. The method of claim 1 or 2, wherein the incubation of the exhausted T cells with the isolated exogenous mitochondria occurs in the presence of rapamycin.

4. The method of claim 3, wherein rapamycin is present at a concentration of 100 nM to 1000 nM.

5. The method of any one of claims 1 to 4, wherein the expression of PD-1 is decreased by at least 1.2 fold.

6. The method of any one of claims 1 to 4, wherein the expression of PD-1 is decreased by at least 1.25 fold.

7. The method of any one of claims 1 to 4, wherein the expression of PD-1 is decreased by at least 1.5 fold.

8. The method of any one of claims 1 to 4, wherein the expression of PD-1 is decreased by at least 2 fold.

9. The method of any one of claims 1 to 4, wherein the expression of PD-1 is decreased by at least 5 fold.

10. The method of any one of claims 1 to 4, wherein the expression of PD-1 is decreased by about 1.1 fold to about 1.5 fold.

11. The method of any one of claims 1 to 10, wherein the method reduces the expression of T-cell immunoglobulin and mucin domain-containing protein 3 (TIM3), lymphocyte-activated gene-3 (LAG3), T Cell immunoglobulin and ITIM domain (TIGIT), TOX, or a combination thereof.

12. The method of any one of claims 1 to 11, wherein the isolated exogenous mitochondria is about 20 μg to about 80 μg protein per 1×10⁶ cells.

13. The method of any one of claims 1 to 12, wherein the mitochondria replaced T cells comprise at least 20% of the exogenous mtDNA.

14. The method of any one of claims 1 to 12, wherein the mitochondria replaced T cells comprises at least 20% of exogenous mtDNA and no more than 80% exogenous mtDNA, as measured by TaqMan Single Nucleotide Polymorphism (SNP) Assay.

15. The method of any one of claims 1 to 14, wherein the sufficient period of time to generate mitochondria replaced T cells is at least approximately 24 hours.

16. The method of any one of claims 1 to 14, wherein the sufficient period of time to generate mitochondria replaced T cells is at least 36 hours.

17. The method of any one of claims 1 to 14, wherein the sufficient period of time to generate mitochondria replaced T cells is at least 48 hours.

18. The method of any one of claims 1 to 14, wherein the sufficient period of time to generate mitochondria replaced T cells is about 24 hours to about 72 hours.

19. The method of any one of claims 1 to 18, wherein the improved effector function comprises increased proliferation, increased cytotoxicity, increased secretion of cytokines, or a combination thereof.

20. The method of any one of claims 1 to 19, wherein the exhausted T cells comprise an exogenous polynucleotide encoding a T cell receptor (TCR) or a chimeric antigen receptor (CAR).

21. The method of any one of claims 1 to 19, wherein the exhausted T cells have been genetically modified to express a T cell receptor (TCR) or a chimeric antigen receptor (CAR).

22. A mitochondria replaced T cell generated by the method of any one of claims 1 to 19.

23. A mitochondria replaced T cell generated by the method of claim 20 or 21.

24. A composition comprising an effective amount of the mitochondria replaced T cell of claim 22, and a pharmaceutically acceptable carrier.

25. A method for ameliorating a symptom of a chronic viral infection in a subject in need thereof, comprising administering to the subject the composition of claim 24.

26. The method of claim 25, wherein the chronic viral infection is a human immunodeficiency virus (HIV) infection, a hepatitis B virus (HBV) infection, a cytomegalovirus infection (CMV), and a Severe Acute respiratory syndrome coronavirus (SARS-COV)-2 infection.

27. A composition comprising an effective amount of the mitochondria replaced T cell of claim 23, and a pharmaceutically acceptable carrier.

28. A method for treating a cancer in a subject in need thereof, comprising administering to the subject the composition of claim 24 or 27.

29. A method for ameliorating a symptom of a cancer in a subject in need thereof, comprising administering to the subject the composition of claim 24 or 27.

30. A method for treating a disease or condition associated with, involving, or caused by T cell exhaustion in a subject in need thereof, comprising administering to the subject the composition of claim 24 or 27, wherein the disease or condition is:
(a) cancer;
(b) a viral infection;
(c) a bacterial infection;
(d) obesity or a metabolic disorder;
(e) alcoholism;
(f) hypermotility;
(g) excessive mental stress;
(h) hypoxia;
(i) an injury;
(j) aging;
(k) aging related immunological dysfunction;
(l) a fibrotic disease;
(m) a macular disease;
(n) a muscular degenerative disease; or
(o) a neurodegenerative disease.

31. A method for ameliorating a symptom of a disease or condition associated with, involving, or caused by T cell exhaustion in a subject in need thereof, comprising administering to the subject the composition of claim 24 or 27, wherein the disease or condition is:
(a) cancer;
(b) a viral infection;
(c) a bacterial infection;
(d) obesity or a metabolic disorder;
(e) alcoholism;
(f) hypermotility;
(g) excessive mental stress;
(h) hypoxia;
(i) an injury;
(i) aging;
(k) aging related immunological dysfunction;
(l) a fibrotic disease;
(m) a macular disease;
(n) a muscular degenerative disease; or
(o) a neurodegenerative disease.

32. A method for treating a disease or condition associated with or involving, or caused by T cell exhaustion in a subject in need thereof, comprising administering to the subject the composition of claim 24 or 27, wherein the disease or condition is:
(a) CD8+ T cell dysfunction;
(b) CD4+ T cell dysfunction;
(c) dysfunction in T cell priming;
(d) memory T cell dysfunction;
(e) effector B cell dysfunction;
(f) dysfunction in B cell priming;
(g) memory B cell dysfunction;
(h) innate lymphoid cell dysfunction;
(i) innate T cell dysfunction; or
(j) innate B cell dysfunction.

33. The method of any one of claims 25, 26, or 28 to 32, wherein the subject is human.


SEQUENCE LISTING (8 sequences)

SEQ ID NO: 1 - protein, 28 aa, Artificial Sequence
  cytochrome c oxidase subunit X (COX 10) MTS
MAASPHTLSSRLLTGCVGGSVWYLERRT

SEQ ID NO: 2 - protein, 24 aa, Artificial Sequence
  cytochrome c oxidase subunit VIII (COX 8) MTS
MSVLTPLLLRSLTGSARRLMVPRA

SEQ ID NO: 3 - DNA, 22 bp, Artificial Sequence
  PCR forward primer
ctctgttctttcatggggaagc

SEQ ID NO: 4 - DNA, 21 bp, Artificial Sequence
  PCR reverse primer
cataaactgtggggggtgtct

SEQ ID NO: 5 - DNA, 20 bp, Artificial Sequence
  forward primer for TaqMan SNP assay
ttactgccagccaccatgaa

SEQ ID NO: 6 - DNA, 24 bp, Artificial Sequence
  reverse primer for TaqMan SNP assay
ttgatgtggattgggtttttatgt

SEQ ID NO: 7 - DNA, 20 bp, Artificial Sequence
  probe for wild type (NHDF) for TaqMan SNP assay
acaggtggttaagtattmgb

SEQ ID NO: 8 - DNA, 18 bp, Artificial Sequence
  probe for the mutation (YG cell) for TaqMan SNP assay
caggtggtcaagtatmgb